(19) United States
(12) Patent Application Publication
(10) Pub. No.: US 2021/0163408 A1
(43) Pub. Date: Jun. 3, 2021

(54) METHOD FOR PREPARING A POLYFLUORINATED COMPOUND

(71) Applicant: ETH ZURICH, Zürich (CH)

(72) Inventors: Cody Ross PITTS, Zuerich (CH); Nico SANTSCHI, Zuerich (CH); Antonio TOGNI, Zuerich (CH)

(73) Assignee: ETH ZURICH, Zürich (CH)

(21) Appl. No.: 17/059,920

(22) PCT Filed: May 29, 2019
(86) PCT No.: PCT/EP2019/063903

§ 371 Date: Nov. 30, 2020

(30) Foreign Application Priority Data
May 31, 2018 (EP) 18175363.3

(51) Int. Cl. C07C 381/00; C07C 391/02; C07C 395/00; C07C 17/013; C07C 25/13; C01B 17/45; C07D 213/803; C07D 209/48; C07D 253/07; C07D 239/30; C07D 213/89; C07D 231/56; C07D 317/16; C07C 67/307; C07C 201/12; C07C 41/22

(52) U.S. Cl. CPC C07C 381/00; C07C 41/22; C07C 395/00; C07C 17/013; C07C 25/13; C01B 17/4507; C07D 213/803; C07D 209/48; C07D 253/07; C07D 239/30; C07D 213/89; C07D 231/56; C07D 317/16; C07C 67/307; C07C 201/12; C07C 391/02

(57) ABSTRACT

A process for preparing a polyfluorinated compound of formula Ar—R₁ (I), wherein Ar—R₁ (I) is an aromatic ring system

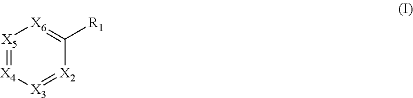

wherein R₁ is selected from the group consisting of SF₄Cl, SF₃, SF₂CF₃, TeF₅, TeF₄CF₃, SeF₃, IF₂, SeF₂CF₃, and IF₄, X₂ is N or CR₂, X₃ is N or CR₃, X₄ is N or CR₄, X₅ is N or CR₅, X₆ is N or CR₆, and the total number of nitrogen atoms in the aromatic ring system is between 0 and 3, wherein R₂, R₃, R₄, R₅ and R₆ are independently selected from the group consisting of hydrogen, fluoro, chloro, bromo, nitro, trifluoromethyl, 2,2,2-trifluoroethyl, pentafluorosulfanyl, phthalimido, azido, benzyloxy, trifluoromethoxy, 2,2,2-trifluoroethoxy, methoxycarbonyl, ethoxycarbonyl, methylcarbonyl, ethylcarbonyl, acetoxy, t-butyl, phenylcarbonyl, benzylcarbonyl, 3-trifluoromethylphenyl, phenylsulfonyl, methylsulfonyl, chlorophenyl, methyldoxolonyl, methyl, isopropyl, cyclopropyl, cyclobutyl, cyclopentyl, cyclohexyl, fluoromethyl, fluoroethyl and phenyl.

The present invention relates to a method for preparing polyfluorinated compounds.

Aromatic ring systems comprising functional groups with polyfluorinated heteroatoms have very promising applications in contemporary medicinal chemistry, agrochemistry, as chemical building blocks, as reagents and for advanced materials, such as liquid crystals.

Historically, synthetic fluorine chemistry has often relied on hazardous reagents and specialized apparatuses. For example, in the case of aryl pentafluorosulfanyl-containing (SF₅) compounds, early reports involved using high-energy reagents such as F₂ or XeF₂. Said reagents are toxic, explosive and corrosive, and the yield of the products obtained when using such high-energy reagents is relatively low. In addition, handling of gas reagents, such as F₂, is expensive when considering their production, storage and use. Alternatively, aryl pentafluorosulfanyl-containing (SF₅) compounds or precursors thereof can be obtained involving SFSCl. Up to now, SF₅Cl is extremely expensive and difficult to obtain.

EP 2 468 720 discloses the synthesis of aryl-SF₅ compounds in a two-step protocol from diaryl disulfides:

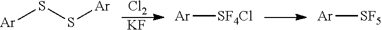

There are several established methods for the second step, i.e. the Cl—F exchange. However, the first step of this procedure, i.e. to access aryl tetrafluoro-λ⁶-sulfanyl chloride compounds (aryl-SF₄Cl), requires handling of chlorine gas in combination with a metal fluoride. Chlorine gas is a very reactive, corrosive reagent and difficult to handle.

US 2005/012072 discloses aryl trifluoromethoxytetrafluoro-sulfuranes, which may be derivatized to yield highly electrically polar molecules.

US 2012/083627 discloses a method of preparing 2,6-dimethyl-4-t-butylphenylsulfur trifluoride by reacting an alkali metal fluoride, bis(2,6-dimethyl-4-t-butylphenyl)disulfide and bromine.

WO 2009/152385 discloses methods for the synthesis of fluoro-sulfur compounds, more specifically of SF₄, SF₅Cl, SF₅Br and SF₆. The method involves admixing Br₂, a metal fluoride reactant, and a sulfur reactant thereby initiating a reaction that produces a yield of the fluoro-sulfur compound of greater than about 10%.

U.S. Pat. No. 3,035,890 discloses a method for preparing SFSCl by reacting ClF₃ with elementary sulfur under anhydrous conditions while maintaining the temperature between 15° C. and 105° C. Chlorine trifluoride is a poisonous, corrosive, and extremely reactive gas.

The problem of the present invention is to provide a method for preparing polyfluorinated compounds without using corrosive and toxic gaseous reagents.

The problem is solved by the method according to the present invention. Further preferred embodiments are subject of the dependent claims.

The process according to the present invention provides a safe method for preparing a polyfluorinated compound of formula

Ar—R₁ (I),

wherein Ar—R₁ (I) is an aromatic ring system

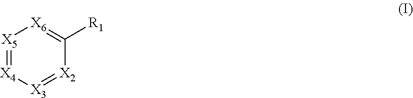

wherein

R₁ is selected from the group consisting of SF₄Cl, SF₃, SF₂CF₃, TeF₅, TeF₄CF₃, SeF₃, SeF₂CF₃, IF₄, and IF₂,

X₂ is N or CR₂,

X₃ is N or CR₃,

X₄ is N or CR₄,

X₅ is N or CR₅,

X₆ is N or CR₆, and

the total number of nitrogen atoms in the aromatic ring system is between 0 and 3,

wherein R₂, R₃, R₄, R₅ and R₆ are independently selected from the group consisting of hydrogen, fluoro, chloro, bromo, nitro, trifluoromethyl, 2,2,2-trifluoroethyl, pentafluorosulfanyl, phthalimido, azido, benzyloxy, trifluoromethoxy, 2,2,2-trifluoroethoxy, methoxycarbonyl, ethoxycarbonyl, methylcarbonyl, ethylcarbonyl, acetoxy, t-butyl, phenylcarbonyl, benzylcarbonyl, 3-trifluoromethylphenyl, phenylsulfonyl, methylsulfonyl, chlorophenyl, methyldoxolonyl, methyl, isopropyl, cyclopropyl, cyclobutyl, cyclopentyl, cyclohexyl, fluoromethyl, fluoroethyl and phenyl,

or if X₅ is CR₅ and X₆ is CR₆ R₅ and R₆ may form together a saturated or unsaturated five or six membered ring system comprising one or more nitrogen, wherein said five or six membered ring system may be substituted with one or more residues R₇ having the same definition as R₂ to R₆, and with the proviso that

if R₁ is SF₃, at least one of R₂ and R₆ is neither hydrogen nor fluoro and

if R₁ is not SF₃, R₂ and R₆ are independently from each other either hydrogen or fluoro and

if at least one of X₂, X₃, X₄, X₅ and X₆ is nitrogen, at least one of R₂, R₃, R₄, R₅ and R₆ is not hydrogen.

Said process involves the following reaction step:

Reacting a starting material selected from the group consisting of Ar₂S₂, Ar₂Te₂, Ar₂Se₂, ArSCF₃, ArTeCF₃, ArI, ArSeCF₃, ArSCH₃, and Ar—SCl, wherein Ar has the same definition as above, and with trichloroisocyanuric acid (TCICA) of the formula (III)

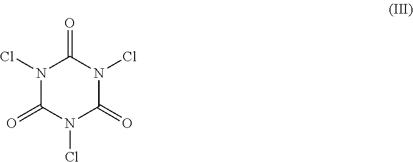 [SMILES: O=c1n(Cl)c(=O)n(Cl)c(=O)n1Cl]

in the presence of an alkali metal fluoride (MF), preferably potassium fluoride (KF).

The method according to the present invention allows a gas reagent-free synthesis of polyfluorinated compounds and in particular of Ar—SF₄Cl compounds in competitive yields using easy-to-handle trichloroisocyanuric acid as an inexpensive oxidant/chlorine source and an alkali metal fluoride. Trichloroisocyanuric acid is a bench-stable, commercially available and cheap solid compound. The method according to the present invention allows the access to a variety of aromatic and heteroaromatic aryl-SF₄Cl compounds in high yields. Said aryl-SF₄Cl compounds can then subsequently be converted to aryl-SF₅ compounds or aryl-SF₄R₁₀ compounds via established synthetic routes. Preferably, the alkali metal fluoride is potassium fluoride due to its lower cost and commercial availability.

In the context of the present invention, the term “aryl” is intended to mean an aromatic ring having six carbon atoms.

In the context of the present invention, the term “heteroaryl” is intended to mean an aryl group where one or more carbon atoms in the aromatic ring have been replaced with one or more nitrogen atoms.

In the context of the present invention, the term “aromatic ring system “Ar”” herein means both, “aryl” and “heteroaryl”.

The method according to the present invention is preferably carried out in presence of a catalytic amount of a Brønsted or Lewis acid. Such a Brønsted or Lewis acid is preferably selected from the group consisting of trifluoroacetic acid (TFA), aluminum chloride (AlCl₃), aluminum bromide (AlBr₃), boron trifluoride (BF₃), tin dichloride (SnCl₂), zinc chloride (ZnCl₂) and titanium tetrachloride (TiCl₄) or a mixture thereof, preferably ZnCl₂ and TFA, most preferably TFA.

Preferably, the Brønsted or Lewis acid, and in particular TFA, is present in the process according to the present invention between 5 mol % and 15 mol %, preferably 10 mol %. Larger quantities of the Brønsted or Lewis acid result in substantial yield loss or complete inhibition of product formation.

Preferably, the molar ratio of TCICA:MF present in the process according to the present invention, is between 1:1 and 1:10, most preferably 1:1 and 1:5, and ideally 1:2 since excessive TCICA can result in additional putative ring chlorination.

Very good results can be obtained for example in reaction conditions comprising 18 equivalents of TCICA, 32 equivalents of the alkali metal fluoride (MF), and 10 mol % of TFA in acetonitrile (MeCN).

Preferably, the method according to the present invention is carried out at room temperature in order to avoid additional ring chlorination which may be observed when heating the reaction mixture to about 45° C. The solvent is preferably a polar aprotic solvent, most preferably selected from the group consisting of ethyl acetate, pivalonitrile and acetonitrile, ideally acetonitrile (MeCN).

Preferably, the metal fluorides, and in particular KF, are dried in advance under inert atmosphere resulting in higher yields than standard MF which have not been dried before using. Most preferably, MF and in particular KF is spray-dried since the consistent particle size distribution positively influences the reaction.

In one embodiment of the present invention, the method relates to the preparation of Ar—R₁ (I), wherein Ar and R₁ have the same definition as above.

Preferably, the process according to the present invention is used to prepare a compound of formula (I), wherein R₁ is SF₄Cl or SF₃, preferably SF₄Cl due to its synthetic importance as chemical building block.

In one embodiment of the present invention, R₁ is SF₄Cl. Aryl- or heteroaryl tetrafluorohalosulfanyl-containing compounds of formula Ar—SF₄Cl (IV) include isomers such as cis-isomers (IVa) and trans-isomers (IVb) as shown below:

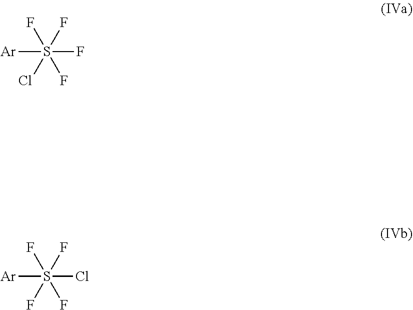

Ar—SF₄Cl is obtained by the method according to the present invention by reacting the corresponding diaryl or heteroaryl disulfide with TCICA and the alkali metal fluoride (MF) (scheme 1). Optionally, a Brønsted or Lewis acid is present as well.

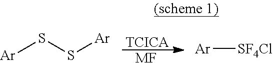

Preferably the alkali metal fluoride is KF. In the aromatic ring system, R₃, R₄, and R₅ are independently selected from the group consisting of hydrogen, fluoro, chloro, bromo, nitro, trifluoromethyl, 2,2,2-trifluoroethyl, pentafluorosulfanyl, phthalimido, azido, benzyloxy, trifluoromethoxy, 2,2,2-trifluoroethoxy, methoxycarbonyl, ethoxycarbonyl, methylcarbonyl, ethylcarbonyl, acetoxy, t-butyl, phenylcarbonyl, benzylcarbonyl, 3-trifluoromethylphenyl, phenylsulfonyl, methylsulfonyl, chlorophenyl, methyldoxolonyl, methyl, isopropyl, cyclopropyl, cyclobutyl, cyclopentyl, cyclohexyl, fluoromethyl, fluoroethyl and phenyl, or if X₅ is CR₅ and X₆ is CR₆ R₅ and R₆ may form together a saturated or unsaturated five or six membered ring system comprising one or more nitrogen, wherein said five or six membered ring system may be substituted with one or more residues R₇ having the same definition as R₂ to R₆, and

R₂ and R₆ are independently from each other either hydrogen or fluoro. Most preferably, R₂ is hydrogen or fluoro and R₆ is hydrogen. Surprisingly, it is also possible to carry out the method according to the present invention if a mild donating group such as a t-butyl group was present in the aromatic ring system. This residue precludes benzylic chlorination and undergoes only minor ring chlorination. Preferably, the aromatic ring system is selected from the group consisting of phenyl, pyridinyl, pyrimidinyl and 2,3,5-triazine, most preferably phenyl.

Ar—SF₄Cl is a very important intermediate product and can be converted into other important synthetic building blocks by a subsequent reaction step, so that the overall reaction is as follows (scheme 2):

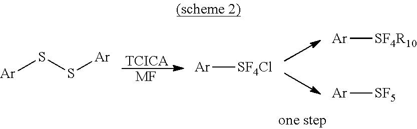

Thus, another embodiment of the present invention relates to the use of Ar—SF₄ as starting material to obtain a compound of formula (V) or (VI)

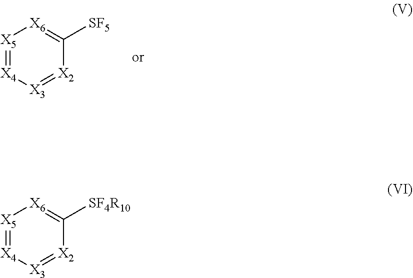

wherein

X₂ is N or CR₂,

X₃ is N or CR₃,

X₄ is N or CR₄,

X₅ is N or CR₅,

X₆ is N or CR₆, and

the total number of nitrogen atoms in the aromatic ring system is between 0 and 3,

R₂ and R₆ are independently from each other either hydrogen or fluoro and

R₃, R₄, and R₅ are independently selected from the group consisting of hydrogen, fluoro, chloro, bromo, nitro, trifluoromethyl, 2,2,2-trifluoroethyl, pentafluorosulfanyl, phthalimido, azido, benzyloxy, trifluoromethoxy, 2,2,2-trifluoroethoxy, methoxycarbonyl, ethoxycarbonyl,

methylcarbonyl, ethylcarbonyl, acetoxy, t-butyl, phenylcarbonyl, benzylcarbonyl, 3-trifluoromethylphenyl, phenylsulfonyl, methylsulfonyl, chlorophenyl, methyldoxolonyl, methyl, isopropyl, cyclopropyl, cyclobutyl, cyclopentyl, cyclohexyl, fluoromethyl, fluoroethyl and phenyl,

or if X₅ is CR₅ and X₆ is CR₆ R₅ and R₆ may form together a saturated or unsaturated five or six membered ring system comprising one or more nitrogen, wherein said five or six membered ring system may be substituted with one or more residues R₇ having the same definition as R₂ to R₆, and R₁₀ is linear or branched, substituted or unsubstituted alkyl, α-alkenyl or α-alkynyl having 2 to 10 carbon atoms.

Thus, one embodiment of the present invention relates to the preparation of the compound of formula (VI) (Ar—SF₄R₁₀). Ar—SF₄Cl obtained by the method according to the present invention can subsequently be converted in a second step to Ar—SF₄R₁₀ by using the well-known BEt₃ chemistry (Das et al, Org. Chem. Front., 2018, 5, 719-724 and Zhong et al, Angew. Chem. Int. Ed., 2014, 53, 526-529). R₁₀ is a linear or branched, substituted or unsubstituted alkyl, α-alkenyl or α-alkynyl group having 2 to 20 carbon atoms. For synthetic reasons, the alkyl or α-alkenyl comprise preferably a chlorine residue in (3-position. The term “α-alkenyl” group stands for an alkenyl group the double bond of which is directly linked to the sulfur atom and the term “α-alkynyl” group stands for an alkynyl group the triple bond of which is directly linked to the sulfur atom.

In case of an alkyl group, R₁₀ is preferably selected from the group consisting of 2-chloro-ethyl, 2-chloro-propyl, 2-chloro-2-phenyl-ethyl, 2-chloro-butyl, 2-chloro-4-phenyl-butyl, 2-chloro-pentyl, 2-chloro-2-cyclohexyl-ethyl, 2-chloro-2-(4-cyclohexylphenyl)-ethyl and 2-chlorohexyl.

In case of an α-alkenyl group, R₁₀ is preferably selected from the group consisting of 2-chloro-ethenyl, 2-chloro-propenyl, 2-chloro-2-phenyl-ethenyl, 2-chloro-butenyl, 2-chloro-4-phenyl-butenyl, 2-chloro-pentenyl, 2-chloro-2-cyclohexyl-ethenyl, 2-chloro-2-(4-cyclohexylphenyl)-ethenyl and 2-chlorohexenyl.

In case of an α-alkynyl group, R₁₀ is preferably selected from the group consisting of ethynyl, propynyl, 3-phenyl-propynyl, 3-cyclohexyl-propynyl, 3-(4-cyclohexylphenyl)-propynyl, butynyl, pentynyl, hexynyl, heptynyl and octynyl.

An α-alkynyl group can be obtained by reacting the corresponding alkyne in the presence of catalytic amounts of BEt₃ and subsequent chloride elimination (scheme 3):

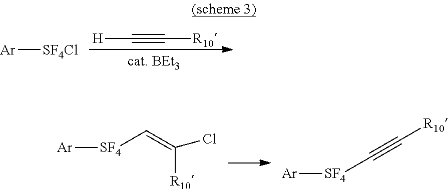

The reaction conditions are known from literature such as in Zhong, L. et al, Angew. Chem. Int. Ed. 2014, 53, 526-529 and Das, P. et al, Org. Chem. Front., 2018, 5, 719-724.

An α-alkenyl group can be obtained by reacting the corresponding alkyne in the presence of catalytic amounts of BEt₃ (scheme 4):

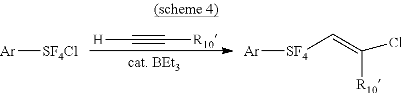

An alkyl group can be obtained by reacting the corresponding alkene in the presence of catalytic amounts of BEt₃ (scheme 5):

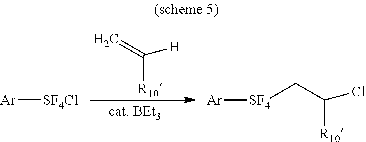

Further, as shown in scheme 6, another embodiment of the present invention relates to the preparation of compounds Ar—R₁, wherein R₁ is SF₅ (formula (IV). Ar—SF₄Cl obtained by the method according to the present invention can be converted to Ar—SF₅ by reacting said compound with silver(I) fluoride at elevated temperature, for example at 120° C. (Kanishchev et al, Angew.

Chem. Int. Ed., 2015, 54, 280-284).

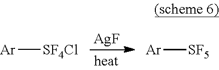

This two-step method for preparing the Ar—SF₅ derivatives significantly reduces the number of synthetic and purification steps from previously reported syntheses. In particular, said reaction step is possible as well if a carbon atom of the ring system is substituted with an acetoxy group, as shown, for example, for the acetoxy group being located in para position of the tetrafluoro-λ6-sulfanyl chloride group (scheme 7).

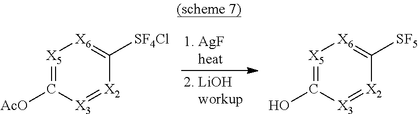

Preferably, a mild saponification procedure such as a LiOH workup of the crude reaction mixture can be carried out to provide direct access to the corresponding (pentafluorosulfanyl)phenol. Thus, said procedure can be generalized to obtain polyfluorinated phenols, hydroxypyridines, hydroxypyrimidines and hydroxytriazines.

Another embodiment of the present invention relates to the production of compounds Ar—R₁, wherein R₁ is SF₃. As the method according to the present invention can also be used to access the S⁺⁴ oxidation state on substrates that contain ortho residues selected from the groups consisting of chloro, brom, nitro, trifluoromethyl, 2,2,2-trifluoroethyl, methoxycarbonyl, ethoxycarbonyl, acetoxy, pentafluorosulfanyl, t-butyl and phenyl (scheme 8). In general, in order to obtain Ar—SF₃, at least one of R₂ or R₆ must not be hydrogen or fluorine.

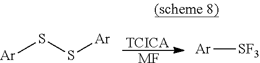

Preferably, R₂ and/or R₆ are electron-withdrawing groups such as chloro, bromo, and nitro. Most preferably, R₂ is chloro or nitro and R₆ is hydrogen. In addition, in the aromatic ring system R₃, R₄, and R₅ are independently selected from the group consisting of hydrogen, fluoro, chloro, bromo, nitro, trifluoromethyl, 2,2,2-trifluoroethyl, methoxycarbonyl, ethoxycarbonyl, acetoxy, pentafluorosulfanyl, t-butyl and phenyl.

Another embodiment of the present invention relates to the preparation of compounds Ar—R₁, wherein R₁ is SF₂CF₃. Ar—SF₂CF₃ is obtained by the method according to the present invention by reacting the corresponding aryl trifluoromethyl sulfide Ar—SCF₃ with TCICA and the alkali metal fluoride (MF) (scheme 9). Optionally, a Brønsted or Lewis acid is present as well.

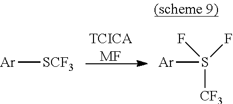

Preferably, the alkali metal fluoride is KF. Ar—SF₂CF₃ may be used as fluorinating agent.

Another embodiment of the present invention relates to the production of compounds Ar—R₁ wherein R₁ is IF₂. Ar—IF₂ is obtained by the method according to the present invention by reacting the corresponding ortho-, meta- or para-substituted aryl iodide Ar—I with TCICA and the alkali metal fluoride (MF) (scheme 10a). Especially good results could be obtained with ortho-substituted aryl iodines. Optionally, a Brønsted or Lewis acid is present as well.

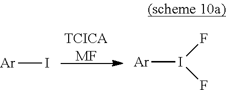

Preferably, the alkali metal fluoride is KF. Ar—IF₂ is an interesting chemical building block and fluorinating reagent.

Alternatively, Ar—I may be used as starting material of the method according to the present invention to prepare Ar—IF₄. Ar—IF₄ is obtained by the method according to the present invention by reacting the corresponding meta- or para-substituted aryl iodide Ar—I with TCICA and the alkali metal fluoride (MF)(scheme 10b). In case of an ortho-substituted aryl iodide, the substitutent in ortho position should be hydrogen or fluoride. Optionally, a Brønsted or Lewis acid is present as well.

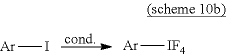

In one embodiment of the present invention, the aromatic ring system of the compound of formula (I) is a substituted or unsubstituted phenyl ring and R₁ to R₆ have the same definition as above (compound of formula (Ia)):

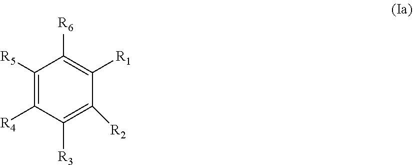

In another embodiment of the present invention, at least one of X₂, X₃, X₄, X₅ and X₆ in the compound of formula (I) is nitrogen, that is, the aromatic ring system is a heteroaromatic ring system. Preferably, exactly one of X₂, X₃, X₄, X₅ and X₆ is nitrogen, that is, the aromatic ring system of the compound of formula (I) is a pyridyl ring and R₂ to R₆ have the same definition as above. Preferably, the nitrogen atom of the pyridine ring system is in position 2 (X₂) (compound of formula (Ib). By substituting the pyridyl ring with an electron-withdrawing group, e.g. a bromine or nitro group, the corresponding heteroaryl-R₁ compounds, in particular heteroaryl-SF₄Cl compounds, are accessible in good yields.

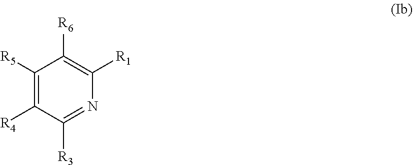

In one embodiment of the present invention, exactly two of X₂, X₃, X₄, X₅ and X₆ in the compound of formula are nitrogen, preferably X₂ and X₆ (compound of formula (Ic)):

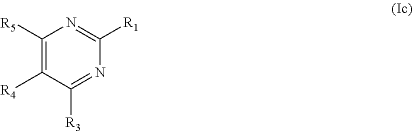

Pyrimidinyl rings substituted with electron-withdrawing groups, e.g. bromine or nitro groups, resulted in the corresponding heteroaryl-R₁ compounds, in particular heteroaryl-SF₄Cl compounds, in good yields as well.

In one embodiment of the present invention, exactly three of X₂, X₃, X₄, X₅ and X₆ are nitrogen, preferably X₂, X₃ and X₆ (compound of formula (Id)):

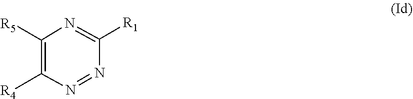

For example, the disulfide derived from 5,6-diphenyl-1,2,4-triazine-3-thiol, resulted in the corresponding 5,6-diphenyl-1,2,4-triazine-3-sulfur chlorotetrafluoride in 67% yield:

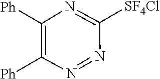

Preferably, in the compound of formula (I) at least one of R₂, R₃, R₄, R₅ and R₆ is fluoro, chloro, bromo, methoxycarbonyl, ethoxycarbonyl or acetoxy, preferably chloro or bromo since it has been shown that the method according to the present invention results in very good yields for aromatic ring systems with electron-withdrawing groups. However, the method according to the present invention does not work in case of free carboxy and free hydroxy groups. Though, this can be circumvented by converting the carboxy group, for instance, to the corresponding methyl ester and ethyl ester or to the corresponding acetal and by converting the hydroxy group, for instance, to the corresponding acetoxy group. Further suitable protecting groups are known to the skilled person. The compatibility of esters under these reaction conditions according to the present invention is a significant advantage over the Cl₂/KF protocol disclosed in EP 2 468 720, which cannot demonstrate the compatibility of esters.

In another embodiment of the present invention, the starting material is a diaryl dichalcogenide selected from the group consisting of Ar₂S₂, Ar₂Te₂ and Ar₂Se₂, preferably Ar₂S₂. Most of the diaryl dichalcogenides are commercially available starting materials which are easy to handle. In particular, diaryl disulfides are common sources of the aryl sulfide units in organic synthesis.

In another embodiment of the present invention, Ar—SF₄Cl can be prepared by using Ar—SCl or Ar—SCH₃ as starting material. One advantage to using either of these starting materials in place of diaryl disulfides lies in synthetic accessibility, as diaryl disulfide substrates with higher molecular weights may be more difficult to synthesize and/or purify.

In another embodiment, the starting material of the method according to the present invention is the diaryl chalcogenide Ar₂Te resulting in a diaryl tetrafluoro-λ6-tellane-compound, which may be used as liquid crystals.

In another embodiment, the starting material of the method according to the present invention is ArSeCF₃ resulting in a difluoro(aryl)(trifluoromethyl)-λ4-selane compound, which may be used, for example, as synthetic building blocks for selenium containing pharmaceuticals. In another embodiment, the starting material of the method according to the present invention is Ar—SCF₃ resulting in Ar—SF₂CF₃ which may be used, for example, as a fluorinating agent.

In another embodiment of the present invention the starting material of the method according to the present invention is ArI since this allows a F₂- and HF-free synthesis of Ar—IF₂ compounds.

Another embodiment of the present invention relates to a safe method for preparing the polyfluorinated compound SF₅Cl (II).

Said process involves the following reaction step:

Reacting the starting material S₈ with trichloroisocyanuric acid (TCICA) of the formula (III)

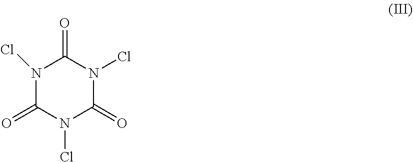 [SMILES: O=c1n(Cl)c(=O)n(Cl)c(=O)n1Cl]

in the presence of an alkali metal fluoride (MF), preferably potassium fluoride (KF). In particular, said process for preparing SF₅Cl is carried out by reacting Se and trichloroisocyanuric acid and the alkali metal fluoride (MF). Optionally, a Brønsted or Lewis acid is present as well. Preferably, the alkali metal fluoride is KF. This synthesis allows the in situ preparation of SF₅Cl which is under normal circumstances extremely difficult to obtain and to handle. The SF₅Cl gas thus obtained can be used to carry out further chemical reaction. Preferably, the SF₅Cl gas thus obtained is directly used for further reaction without purification.

Another embodiment of the present invention relates to a safe method for preparing the polyfluorinated compound CF₃SF₄Cl. Said process involves the following reaction step:

Reacting the starting material Ar—S—S—CF₃, wherein Ar has the same definition as above, with trichloroisocyanuric acid (TCICA) of the formula (III)

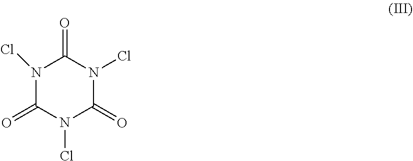 [SMILES: O=c1n(Cl)c(=O)n(Cl)c(=O)n1Cl]

in the presence of an alkali metal fluoride (MF), preferably potassium fluoride (KF). Preferably, Ar is phenyl or a para-nitro-phenyl. In particular, said process for preparing CF₃SF₄Cl is carried out by reacting Ar—S—S—CF₃ and trichloroisocyanuric acid and the alkali metal fluoride (MF). Optionally, a Brønsted or Lewis acid is present as well. Preferably, the alkali metal fluoride is KF. This synthesis allows the in situ preparation of CF₃SF₄Cl. The CF₃SF₄Cl gas thus obtained can be used to carry out further chemical reaction, in particular for the preparation of novel materials or biologically active agents comprising this extraordinarily lipophilic and profoundly electron withdrawing group. Preferably, the CF₃SF₄Cl gas thus obtained is directly used for further reaction without purification.

By the method of the present invention the following compounds of formula (I)

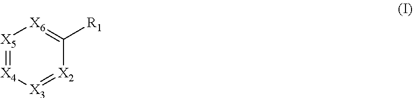

may preferably be obtained in a very easy way:

| R₁ | X₂ | X₃ | X₄ | X₅ | X₆ | R₁ | X₂ | X₃ | X₄ | X₅ | X₆ |
|---|---|---|---|---|---|---|---|---|---|---|---|
| SF₄Cl | C—H | C—H | C—H | C—H | C—H | TeF₅ | C—H | C—H | C—H | C—H | C—H |
| SF₄Cl | C—H | C—F | C—H | C—H | C—H | TeF₅ | C—H | C—F | C—H | C—H | C—H |
| SF₄Cl | C—H | C—Cl | C—H | C—H | C—H | TeF₅ | C—H | C—Cl | C—H | C—H | C—H |
| SF₄Cl | C—H | C—Br | C—H | C—H | C—H | TeF₅ | C—H | C—Br | C—H | C—H | C—H |
| SF₄Cl | C—H | C—NO₂ | C—H | C—H | C—H | TeF₅ | C—H | C—NO₂ | C—H | C—H | C—H |
| SF₄Cl | C—H | C—CF₃ | C—H | C—H | C—H | TeF₅ | C—H | C—CF₃ | C—H | C—H | C—H |
| SF₄Cl | C—H | C—COOMe | C—H | C—H | C—H | TeF₅ | C—H | C—COOMe | C—H | C—H | C—H |
| SF₄Cl | C—H | C—COOEt | C—H | C—H | C—H | TeF₅ | C—H | C—COOEt | C—H | C—H | C—H |
| SF₄Cl | C—H | C—OAc | C—H | C—H | C—H | TeF₅ | C—H | C—OAc | C—H | C—H | C—H |
| SF₄Cl | C—H | C—SF₅ | C—H | C—H | C—H | TeF₅ | C—H | C—SF₅ | C—H | C—H | C—H |
| SF₄Cl | C—H | C—tBu | C—H | C—H | C—H | TeF₅ | C—H | C—tBu | C—H | C—H | C—H |
| SF₄Cl | C—H | C—Ph | C—H | C—H | C—H | TeF₅ | C—H | C—Ph | C—H | C—H | C—H |
| SF₄Cl | C—F | C—H | C—H | C—H | C—H | TeF₅ | C—F | C—H | C—H | C—H | C—H |
| SF₄Cl | C—F | C—F | C—H | C—H | C—H | TeF₅ | C—F | C—F | C—H | C—H | C—H |
| SF₄Cl | C—F | C—Cl | C—H | C—H | C—H | TeF₅ | C—F | C—Cl | C—H | C—H | C—H |
| SF₄Cl | C—F | C—Br | C—H | C—H | C—H | TeF₅ | C—F | C—Br | C—H | C—H | C—H |
| SF₄Cl | C—F | C—NO₂ | C—H | C—H | C—H | TeF₅ | C—F | C—NO₂ | C—H | C—H | C—H |
| SF₄Cl | C—F | C—CF₃ | C—H | C—H | C—H | TeF₅ | C—F | C—CF₃ | C—H | C—H | C—H |
| SF₄Cl | C—F | C—COOMe | C—H | C—H | C—H | TeF₅ | C—F | C—COOMe | C—H | C—H | C—H |
| SF₄Cl | C—F | C—COOEt | C—H | C—H | C—H | TeF₅ | C—F | C—COOEt | C—H | C—H | C—H |
| SF₄Cl | C—F | C—OAc | C—H | C—H | C—H | TeF₅ | C—F | C—OAc | C—H | C—H | C—H |
| SF₄Cl | C—F | C—SF₅ | C—H | C—H | C—H | TeF₅ | C—F | C—SF₅ | C—H | C—H | C—H |
| SF₄Cl | C—F | C—tBu | C—H | C—H | C—H | TeF₅ | C—F | C—tBu | C—H | C—H | C—H |
| SF₄Cl | C—F | C—Ph | C—H | C—H | C—H | TeF₅ | C—F | C—Ph | C—H | C—H | C—H |
| SF₄Cl | C—H | C—H | C—H | C—H | C—H | TeF₅ | C—H | C—H | C—H | C—H | C—H |
| SF₄Cl | C—H | C—H | C—F | C—H | C—H | TeF₅ | C—H | C—H | C—F | C—H | C—H |
| SF₄Cl | C—H | C—H | C—Cl | C—H | C—H | TeF₅ | C—H | C—H | C—Cl | C—H | C—H |
| SF₄Cl | C—H | C—H | C—Br | C—H | C—H | TeF₅ | C—H | C—H | C—Br | C—H | C—H |
| SF₄Cl | C—H | C—H | C—NO₂ | C—H | C—H | TeF₅ | C—H | C—H | C—NO₂ | C—H | C—H |
| SF₄Cl | C—H | C—H | C—CF₃ | C—H | C—H | TeF₅ | C—H | C—H | C—CF₃ | C—H | C—H |
| SF₄Cl | C—H | C—H | C—COOMe | C—H | C—H | TeF₅ | C—H | C—H | C—COOMe | C—H | C—H |
| SF₄Cl | C—H | C—H | C—COOEt | C—H | C—H | TeF₅ | C—H | C—H | C—COOEt | C—H | C—H |
| SF₄Cl | C—H | C—H | C—OAc | C—H | C—H | TeF₅ | C—H | C—H | C—OAc | C—H | C—H |
| SF₄Cl | C—H | C—H | C—SF₅ | C—H | C—H | TeF₅ | C—H | C—H | C—SF₅ | C—H | C—H |
| SF₄Cl | C—H | C—H | C—tBu | C—H | C—H | TeF₅ | C—H | C—H | C—tBu | C—H | C—H |
| SF₄Cl | C—H | C—H | C—Ph | C—H | C—H | TeF₅ | C—H | C—H | C—Ph | C—H | C—H |
| SF₄Cl | C—F | C—H | C—F | C—H | C—H | TeF₅ | C—F | C—H | C—F | C—H | C—H |
| SF₄Cl | C—F | C—H | C—Cl | C—H | C—H | TeF₅ | C—F | C—H | C—Cl | C—H | C—H |
| SF₄Cl | C—F | C—H | C—Br | C—H | C—H | TeF₅ | C—F | C—H | C—Br | C—H | C—H |
| SF₄Cl | C—F | C—H | C—NO₂ | C—H | C—H | TeF₅ | C—F | C—H | C—NO₂ | C—H | C—H |
| SF₄Cl | C—F | C—H | C—CF₃ | C—H | C—H | TeF₅ | C—F | C—H | C—CF₃ | C—H | C—H |
| SF₄Cl | C—F | C—H | C—COOMe | C—H | C—H | TeF₅ | C—F | C—H | C—COOMe | C—H | C—H |
| SF₄Cl | C—F | C—H | C—COOEt | C—H | C—H | TeF₅ | C—F | C—H | C—COOEt | C—H | C—H |
| SF₄Cl | C—F | C—H | C—OAc | C—H | C—H | TeF₅ | C—F | C—H | C—OAc | C—H | C—H |
| SF₄Cl | C—F | C—H | C—SF₅ | C—H | C—H | TeF₅ | C—F | C—H | C—SF₅ | C—H | C—H |
| SF₄Cl | C—F | C—H | C—tBu | C—H | C—H | TeF₅ | C—F | C—H | C—tBu | C—H | C—H |
| SF₄Cl | C—F | C—H | C—Ph | C—H | C—H | TeF₅ | C—F | C—H | C—Ph | C—H | C—H |
| SF₄Cl | C—H | C—F | C—F | C—H | C—H | TeF₅ | C—H | C—F | C—F | C—H | C—H |
| SF₄Cl | C—H | C—F | C—Cl | C—H | C—H | TeF₅ | C—H | C—F | C—Cl | C—H | C—H |
| SF₄Cl | C—H | C—F | C—Br | C—H | C—H | TeF₅ | C—H | C—F | C—Br | C—H | C—H |
| SF₄Cl | C—H | C—Cl | C—F | C—H | C—H | TeF₅ | C—H | C—Cl | C—F | C—H | C—H |
| SF₄Cl | C—H | C—Cl | C—Cl | C—H | C—H | TeF₅ | C—H | C—Cl | C—Cl | C—H | C—H |
| SF₄Cl | C—H | C—Cl | C—Br | C—H | C—H | TeF₅ | C—H | C—Cl | C—Br | C—H | C—H |
| SF₄Cl | C—H | C—Br | C—F | C—H | C—H | TeF₅ | C—H | C—Br | C—F | C—H | C—H |
| SF₄Cl | C—H | C—Br | C—Cl | C—H | C—H | TeF₅ | C—H | C—Br | C—Cl | C—H | C—H |
| SF₄Cl | C—H | C—Br | C—Cl | C—H | C—H | TeF₅ | C—H | C—Br | C—Cl | C—H | C—H |
| SF₄Cl | C—H | C—NO₂ | C—F | C—H | C—H | TeF₅ | C—H | C—NO₂ | C—F | C—H | C—H |
| SF₄Cl | C—H | C—NO₂ | C—Cl | C—H | C—H | TeF₅ | C—H | C—NO₂ | C—Cl | C—H | C—H |
| SF₄Cl | C—H | C—NO₂ | C—Br | C—H | C—H | TeF₅ | C—H | C—NO₂ | C—Br | C—H | C—H |
| SF₄Cl | C—H | C—NO₂ | C—COOMe | C—H | C—H | TeF₅ | C—H | C—NO₂ | C—COOMe | C—H | C—H |
| SF₄Cl | C—H | C—NO₂ | C—COOEt | C—H | C—H | TeF₅ | C—H | C—NO₂ | C—COOEt | C—H | C—H |
| SF₄Cl | C—H | C—F | C—NO₂ | C—H | C—H | TeF₅ | C—H | C—F | C—NO₂ | C—H | C—H |
| SF₄Cl | C—H | C—Cl | C—NO₂ | C—H | C—H | TeF₅ | C—H | C—Cl | C—NO₂ | C—H | C—H |
| SF₄Cl | C—H | C—Br | C—NO₂ | C—H | C—H | TeF₅ | C—H | C—Br | C—NO₂ | C—H | C—H |
| SF₄Cl | C—H | C—COOMe | C—NO₂ | C—H | C—H | TeF₅ | C—H | C—COOMe | C—NO₂ | C—H | C—H |
| SF₄Cl | C—H | C—COOEt | C—NO₂ | C—H | C—H | TeF₅ | C—H | C—COOEt | C—NO₂ | C—H | C—H |
| SF₄Cl | C—H | C—CF₃ | C—F | C—H | C—H | TeF₅ | C—H | C—CF₃ | C—F | C—H | C—H |
| SF₄Cl | C—H | C—CF₃ | C—Cl | C—H | C—H | TeF₅ | C—H | C—CF₃ | C—Cl | C—H | C—H |
| SF₄Cl | C—H | C—CF₃ | C—Br | C—H | C—H | TeF₅ | C—H | C—CF₃ | C—Br | C—H | C—H |
| SF₄Cl | C—H | C—CF₃ | C—OAc | C—H | C—H | TeF₅ | C—H | C—CF₃ | C—OAc | C—H | C—H |
| SF₄Cl | C—H | C—CF₃ | C—NO₂ | C—H | C—H | TeF₅ | C—H | C—CF₃ | C—NO₂ | C—H | C—H |
| SF₄Cl | C—H | C—NPhth | C—H | C—H | C—H | TeF₅ | C—H | C—NPhth | C—H | C—H | C—H |
| SF₄Cl | C—H | C—H | C—NPhth | C—H | C—H | TeF₅ | C—H | C—H | C—NPhth | C—H | C—H |
| SF₄Cl | C—H | C—OBz | C—H | C—H | C—H | TeF₅ | C—H | C—OBz | C—H | C—H | C—H |
| SF₄Cl | C—H | C—H | C—OBz | C—H | C—H | TeF₅ | C—H | C—H | C—OBz | C—H | C—H |
| SF₄Cl | C—H | C—N₃ | C—H | C—H | C—H | TeF₅ | C—H | C—N₃ | C—H | C—H | C—H |
| SF₄Cl | C—H | C—H | C—N₃ | C—H | C—H | TeF₅ | C—H | C—H | C—N₃ | C—H | C—H |
| SF₄Cl | C—F | C—F | C—CF₃ | C—H | C—H | TeF₅ | C—F | C—F | C—CF₃ | C—H | C—H |
| SF₄Cl | C—F | C—Cl | C—CF₃ | C—H | C—H | TeF₅ | C—F | C—Cl | C—CF₃ | C—H | C—H |
| SF₄Cl | C—F | C—Br | C—CF₃ | C—H | C—H | TeF₅ | C—F | C—Br | C—CF₃ | C—H | C—H |
| SF₄Cl | C—F | C—OAc | C—CF₃ | C—H | C—H | TeF₅ | C—F | C—OAc | C—CF₃ | C—H | C—H |
| SF₄Cl | C—F | C—NO₂ | C—CF₃ | C—H | C—H | TeF₅ | C—F | C—NO₂ | C—CF₃ | C—H | C—H |
| SF₄Cl | C—F | C—F | C—F | C—H | C—H | TeF₅ | C—F | C—F | C—F | C—H | C—H |
| SF₄Cl | C—F | C—F | C—Cl | C—H | C—H | TeF₅ | C—F | C—F | C—Cl | C—H | C—H |
| SF₄Cl | C—F | C—F | C—Br | C—H | C—H | TeF₅ | C—F | C—F | C—Br | C—H | C—H |
| SF₄Cl | C—F | C—Cl | C—F | C—H | C—H | TeF₅ | C—F | C—Cl | C—F | C—H | C—H |
| SF₄Cl | C—F | C—Cl | C—Cl | C—H | C—H | TeF₅ | C—F | C—Cl | C—Cl | C—H | C—H |
| SF₄Cl | C—F | C—Cl | C—Br | C—H | C—H | TeF₅ | C—F | C—Cl | C—Br | C—H | C—H |
| SF₄Cl | C—F | C—Br | C—F | C—H | C—H | TeF₅ | C—F | C—Br | C—F | C—H | C—H |
| SF₄Cl | C—F | C—Br | C—Cl | C—H | C—H | TeF₅ | C—F | C—Br | C—Cl | C—H | C—H |
| SF₄Cl | C—F | C—Br | C—Cl | C—H | C—H | TeF₅ | C—F | C—Br | C—Cl | C—H | C—H |
| SF₄Cl | C—F | C—NO₂ | C—F | C—H | C—H | TeF₅ | C—F | C—NO₂ | C—F | C—H | C—H |
| SF₄Cl | C—F | C—NO₂ | C—Cl | C—H | C—H | TeF₅ | C—F | C—NO₂ | C—Cl | C—H | C—H |
| SF₄Cl | C—F | C—NO₂ | C—Br | C—H | C—H | TeF₅ | C—F | C—NO₂ | C—Br | C—H | C—H |
| SF₄Cl | C—F | C—NO₂ | C—COOMe | C—H | C—H | TeF₅ | C—F | C—NO₂ | C—COOMe | C—H | C—H |
| SF₄Cl | C—F | C—NO₂ | C—COOEt | C—H | C—H | TeF₅ | C—F | C—NO₂ | C—COOEt | C—H | C—H |
| SF₄Cl | C—F | C—F | C—NO₂ | C—H | C—H | TeF₅ | C—F | C—F | C—NO₂ | C—H | C—H |
| SF₄Cl | C—F | C—Cl | C—NO₂ | C—H | C—H | TeF₅ | C—F | C—Cl | C—NO₂ | C—H | C—H |
| SF₄Cl | C—F | C—Br | C—NO₂ | C—H | C—H | TeF₅ | C—F | C—Br | C—NO₂ | C—H | C—H |
| SF₄Cl | C—F | C—COOMe | C—NO₂ | C—H | C—H | TeF₅ | C—F | C—COOMe | C—NO₂ | C—H | C—H |
| SF₄Cl | C—F | C—COOEt | C—NO₂ | C—H | C—H | TeF₅ | C—F | C—COOEt | C—NO₂ | C—H | C—H |
| SF₄Cl | C—F | C—CF₃ | C—F | C—H | C—H | TeF₅ | C—F | C—CF₃ | C—F | C—H | C—H |
| SF₄Cl | C—F | C—CF₃ | C—Cl | C—H | C—H | TeF₅ | C—F | C—CF₃ | C—Cl | C—H | C—H |
| SF₄Cl | C—F | C—CF₃ | C—Br | C—H | C—H | TeF₅ | C—F | C—CF₃ | C—Br | C—H | C—H |
| SF₄Cl | C—F | C—CF₃ | C—OAc | C—H | C—H | TeF₅ | C—F | C—CF₃ | C—OAc | C—H | C—H |
| SF₄Cl | C—F | C—CF₃ | C—NO₂ | C—H | C—H | TeF₅ | C—F | C—CF₃ | C—NO₂ | C—H | C—H |
| SF₄Cl | C—F | C—F | C—CF₃ | C—H | C—H | TeF₅ | C—F | C—F | C—CF₃ | C—H | C—H |
| SF₄Cl | C—F | C—Cl | C—CF₃ | C—H | C—H | TeF₅ | C—F | C—Cl | C—CF₃ | C—H | C—H |
| SF₄Cl | C—F | C—Br | C—CF₃ | C—H | C—H | TeF₅ | C—F | C—Br | C—CF₃ | C—H | C—H |
| SF₄Cl | C—F | C—OAc | C—CF₃ | C—H | C—H | TeF₅ | C—F | C—OAc | C—CF₃ | C—H | C—H |
| SF₄Cl | C—F | C—NO₂ | C—CF₃ | C—H | C—H | TeF₅ | C—F | C—NO₂ | C—CF₃ | C—H | C—H |
| SF₄Cl | C—F | C—NPhth | C—H | C—H | C—H | TeF₅ | C—F | C—NPhth | C—H | C—H | C—H |
| SF₄Cl | C—F | C—H | C—NPhth | C—H | C—H | TeF₅ | C—F | C—H | C—NPhth | C—H | C—H |
| SF₄Cl | C—F | C—H | C—H | C—NPhth | C—H | TeF₅ | C—F | C—H | C—H | C—NPhth | C—H |
| SF₄Cl | C—F | C—OBz | C—H | C—H | C—H | TeF₅ | C—F | C—OBz | C—H | C—H | C—H |
| SF₄Cl | C—F | C—H | C—OBz | C—H | C—H | TeF₅ | C—F | C—H | C—OBz | C—H | C—H |
| SF₄Cl | C—F | C—H | C—H | C—OBz | C—H | TeF₅ | C—F | C—H | C—H | C—OBz | C—H |
| SF₄Cl | C—F | C—N₃ | C—H | C—H | C—H | TeF₅ | C—F | C—N₃ | C—H | C—H | C—H |
| SF₄Cl | C—F | C—H | C—N₃ | C—H | C—H | TeF₅ | C—F | C—H | C—N₃ | C—H | C—H |
| SF₄Cl | C—F | C—H | C—H | C—N₃ | C—H | TeF₅ | C—F | C—H | C—H | C—N₃ | C—H |
| SF₄Cl | N | C—F | C—H | C—H | C—H | TeF₅ | N | C—F | C—H | C—H | C—H |
| SF₄Cl | N | C—Cl | C—H | C—H | C—H | TeF₅ | N | C—Cl | C—H | C—H | C—H |
| SF₄Cl | N | C—Br | C—H | C—H | C—H | TeF₅ | N | C—Br | C—H | C—H | C—H |
| SF₄Cl | N | C—NO₂ | C—H | C—H | C—H | TeF₅ | N | C—NO₂ | C—H | C—H | C—H |
| SF₄Cl | N | C—CF₃ | C—H | C—H | C—H | TeF₅ | N | C—CF₃ | C—H | C—H | C—H |
| SF₄Cl | N | C—COOMe | C—H | C—H | C—H | TeF₅ | N | C—COOMe | C—H | C—H | C—H |
| SF₄Cl | N | C—COOEt | C—H | C—H | C—H | TeF₅ | N | C—COOEt | C—H | C—H | C—H |
| SF₄Cl | N | C—OAc | C—H | C—H | C—H | TeF₅ | N | C—OAc | C—H | C—H | C—H |
| SF₄Cl | N | C—SF₅ | C—H | C—H | C—H | TeF₅ | N | C—SF₅ | C—H | C—H | C—H |
| SF₄Cl | N | C—tBu | C—H | C—H | C—H | TeF₅ | N | C—tBu | C—H | C—H | C—H |
| SF₄Cl | N | C—Ph | C—H | C—H | C—H | TeF₅ | N | C—Ph | C—H | C—H | C—H |
| SF₄Cl | N | C—H | C—H | C—H | C—H | TeF₅ | N | C—H | C—H | C—H | C—H |
| SF₄Cl | N | C—H | C—H | C—H | C—H | TeF₅ | N | C—H | C—H | C—H | C—H |
| SF₄Cl | N | C—H | C—F | C—H | C—H | TeF₅ | N | C—H | C—F | C—H | C—H |
| SF₄Cl | N | C—H | C—Cl | C—H | C—H | TeF₅ | N | C—H | C—Cl | C—H | C—H |
| SF₄Cl | N | C—H | C—Br | C—H | C—H | TeF₅ | N | C—H | C—Br | C—H | C—H |
| SF₄Cl | N | C—H | C—NO₂ | C—H | C—H | TeF₅ | N | C—H | C—NO₂ | C—H | C—H |
| SF₄Cl | N | C—H | C—CF₃ | C—H | C—H | TeF₅ | N | C—H | C—CF₃ | C—H | C—H |
| SF₄Cl | N | C—H | C—COOMe | C—H | C—H | TeF₅ | N | C—H | C—COOMe | C—H | C—H |
| SF₄Cl | N | C—H | C—COOEt | C—H | C—H | TeF₅ | N | C—H | C—COOEt | C—H | C—H |
| SF₄Cl | N | C—H | C—OAc | C—H | C—H | TeF₅ | N | C—H | C—OAc | C—H | C—H |
| SF₄Cl | N | C—H | C—SF₅ | C—H | C—H | TeF₅ | N | C—H | C—SF₅ | C—H | C—H |
| SF₄Cl | N | C—H | C—tBu | C—H | C—H | TeF₅ | N | C—H | C—tBu | C—H | C—H |
| SF₄Cl | N | C—H | C—Ph | C—H | C—H | TeF₅ | N | C—H | C—Ph | C—H | C—H |
| SF₄Cl | N | C—F | C—F | C—H | C—H | TeF₅ | N | C—F | C—F | C—H | C—H |
| SF₄Cl | N | C—F | C—Cl | C—H | C—H | TeF₅ | N | C—F | C—Cl | C—H | C—H |
| SF₄Cl | N | C—F | C—Br | C—H | C—H | TeF₅ | N | C—F | C—Br | C—H | C—H |
| SF₄Cl | N | C—Cl | C—F | C—H | C—H | TeF₅ | N | C—Cl | C—F | C—H | C—H |
| SF₄Cl | N | C—Cl | C—Cl | C—H | C—H | TeF₅ | N | C—Cl | C—Cl | C—H | C—H |
| SF₄Cl | N | C—Cl | C—Br | C—H | C—H | TeF₅ | N | C—Cl | C—Br | C—H | C—H |
| SF₄Cl | N | C—Br | C—F | C—H | C—H | TeF₅ | N | C—Br | C—F | C—H | C—H |
| SF₄Cl | N | C—Br | C—Cl | C—H | C—H | TeF₅ | N | C—Br | C—Cl | C—H | C—H |
| SF₄Cl | N | C—Br | C—Cl | C—H | C—H | TeF₅ | N | C—Br | C—Cl | C—H | C—H |
| SF₄Cl | N | C—NO₂ | C—F | C—H | C—H | TeF₅ | N | C—NO₂ | C—F | C—H | C—H |
| SF₄Cl | N | C—NO₂ | C—Cl | C—H | C—H | TeF₅ | N | C—NO₂ | C—Cl | C—H | C—H |
| SF₄Cl | N | C—NO₂ | C—Br | C—H | C—H | TeF₅ | N | C—NO₂ | C—Br | C—H | C—H |
| SF₄Cl | N | C—NO₂ | C—COOMe | C—H | C—H | TeF₅ | N | C—NO₂ | C—COOMe | C—H | C—H |
| SF₄Cl | N | C—NO₂ | C—COOEt | C—H | C—H | TeF₅ | N | C—NO₂ | C—COOEt | C—H | C—H |
| SF₄Cl | N | C—F | C—NO₂ | C—H | C—H | TeF₅ | N | C—F | C—NO₂ | C—H | C—H |
| SF₄Cl | N | C—Cl | C—NO₂ | C—H | C—H | TeF₅ | N | C—Cl | C—NO₂ | C—H | C—H |
| SF₄Cl | N | C—Br | C—NO₂ | C—H | C—H | TeF₅ | N | C—Br | C—NO₂ | C—H | C—H |
| SF₄Cl | N | C—COOMe | C—NO₂ | C—H | C—H | TeF₅ | N | C—COOMe | C—NO₂ | C—H | C—H |
| SF₄Cl | N | C—COOEt | C—NO₂ | C—H | C—H | TeF₅ | N | C—COOEt | C—NO₂ | C—H | C—H |
| SF₄Cl | N | C—CF₃ | C—F | C—H | C—H | TeF₅ | N | C—CF₃ | C—F | C—H | C—H |
| SF₄Cl | N | C—CF₃ | C—Cl | C—H | C—H | TeF₅ | N | C—CF₃ | C—Cl | C—H | C—H |
| SF₄Cl | N | C—CF₃ | C—Br | C—H | C—H | TeF₅ | N | C—CF₃ | C—Br | C—H | C—H |
| SF₄Cl | N | C—CF₃ | C—OAc | C—H | C—H | TeF₅ | N | C—CF₃ | C—OAc | C—H | C—H |
| SF₄Cl | N | C—CF₃ | C—NO₂ | C—H | C—H | TeF₅ | N | C—CF₃ | C—NO₂ | C—H | C—H |
| SF₄Cl | N | C—NPhth | C—H | C—H | C—H | TeF₅ | N | C—NPhth | C—H | C—H | C—H |
| SF₄Cl | N | C—H | C—NPhth | C—H | C—H | TeF₅ | N | C—H | C—NPhth | C—H | C—H |
| SF₄Cl | N | C—H | C—H | C—NPhth | C—H | TeF₅ | N | C—H | C—H | C—NPhth | C—H |
| SF₄Cl | N | C—OBz | C—H | C—H | C—H | TeF₅ | N | C—OBz | C—H | C—H | C—H |
| SF₄Cl | N | C—H | C—OBz | C—H | C—H | TeF₅ | N | C—H | C—OBz | C—H | C—H |
| SF₄Cl | N | C—H | C—H | C—OBz | C—H | TeF₅ | N | C—H | C—H | C—OBz | C—H |
| SF₄Cl | N | C—N₃ | C—H | C—H | C—H | TeF₅ | N | C—N₃ | C—H | C—H | C—H |
| SF₄Cl | N | C—H | C—N₃ | C—H | C—H | TeF₅ | N | C—H | C—N₃ | C—H | C—H |
| SF₄Cl | N | C—H | C—H | C—N₃ | C—H | TeF₅ | N | C—H | C—H | C—N₃ | C—H |
| SF₄Cl | N | C—F | C—H | C—H | N | TeF₅ | N | C—F | C—H | C—H | N |
| SF₄Cl | N | C—Cl | C—H | C—H | N | TeF₅ | N | C—Cl | C—H | C—H | N |
| SF₄Cl | N | C—Br | C—H | C—H | N | TeF₅ | N | C—Br | C—H | C—H | N |
| SF₄Cl | N | C—NO₂ | C—H | C—H | N | TeF₅ | N | C—NO₂ | C—H | C—H | N |
| SF₄Cl | N | C—CF₃ | C—H | C—H | N | TeF₅ | N | C—CF₃ | C—H | C—H | N |
| SF₄Cl | N | C—COOMe | C—H | C—H | N | TeF₅ | N | C—COOMe | C—H | C—H | N |
| SF₄Cl | N | C—COOEt | C—H | C—H | N | TeF₅ | N | C—COOEt | C—H | C—H | N |
| SF₄Cl | N | C—OAc | C—H | C—H | N | TeF₅ | N | C—OAc | C—H | C—H | N |
| SF₄Cl | N | C—SF₅ | C—H | C—H | N | TeF₅ | N | C—SF₅ | C—H | C—H | N |
| SF₄Cl | N | C—tBu | C—H | C—H | N | TeF₅ | N | C—tBu | C—H | C—H | N |
| SF₄Cl | N | C—Ph | C—H | C—H | N | TeF₅ | N | C—Ph | C—H | C—H | N |
| SF₄Cl | N | C—H | C—F | C—H | N | TeF₅ | N | C—H | C—F | C—H | N |
| SF₄Cl | N | C—H | C—Cl | C—H | N | TeF₅ | N | C—H | C—Cl | C—H | N |
| SF₄Cl | N | C—H | C—Br | C—H | N | TeF₅ | N | C—H | C—Br | C—H | N |
| SF₄Cl | N | C—H | C—NO₂ | C—H | N | TeF₅ | N | C—H | C—NO₂ | C—H | N |
| SF₄Cl | N | C—H | C—CF₃ | C—H | N | TeF₅ | N | C—H | C—CF₃ | C—H | N |
| SF₄Cl | N | C—H | C—COOMe | C—H | N | TeF₅ | N | C—H | C—COOMe | C—H | N |
| SF₄Cl | N | C—H | C—COOEt | C—H | N | TeF₅ | N | C—H | C—COOEt | C—H | N |
| SF₄Cl | N | C—H | C—OAc | C—H | N | TeF₅ | N | C—H | C—OAc | C—H | N |
| SF₄Cl | N | C—H | C—SF₅ | C—H | N | TeF₅ | N | C—H | C—SF₅ | C—H | N |
| SF₄Cl | N | C—H | C—tBu | C—H | N | TeF₅ | N | C—H | C—tBu | C—H | N |
| SF₄Cl | N | C—H | C—Ph | C—H | N | TeF₅ | N | C—H | C—Ph | C—H | N |
| SF₄Cl | N | C—F | C—F | C—H | N | TeF₅ | N | C—F | C—F | C—H | N |
| SF₄Cl | N | C—F | C—Cl | C—H | N | TeF₅ | N | C—F | C—Cl | C—H | N |
| SF₄Cl | N | C—F | C—Br | C—H | N | TeF₅ | N | C—F | C—Br | C—H | N |
| SF₄Cl | N | C—Cl | C—F | C—H | N | TeF₅ | N | C—Cl | C—F | C—H | N |
| SF₄Cl | N | C—Cl | C—Cl | C—H | N | TeF₅ | N | C—Cl | C—Cl | C—H | N |
| SF₄Cl | N | C—Cl | C—Br | C—H | N | TeF₅ | N | C—Cl | C—Br | C—H | N |
| SF₄Cl | N | C—Br | C—F | C—H | N | TeF₅ | N | C—Br | C—F | C—H | N |
| SF₄Cl | N | C—Br | C—Cl | C—H | N | TeF₅ | N | C—Br | C—Cl | C—H | N |
| SF₄Cl | N | C—Br | C—Cl | C—H | N | TeF₅ | N | C—Br | C—Cl | C—H | N |
| SF₄Cl | N | C—NO₂ | C—F | C—H | N | TeF₅ | N | C—NO₂ | C—F | C—H | N |
| SF₄Cl | N | C—NO₂ | C—Cl | C—H | N | TeF₅ | N | C—NO₂ | C—Cl | C—H | N |
| SF₄Cl | N | C—NO₂ | C—Br | C—H | N | TeF₅ | N | C—NO₂ | C—Br | C—H | N |
| SF₄Cl | N | C—NO₂ | C—COOMe | C—H | N | TeF₅ | N | C—NO₂ | C—COOMe | C—H | N |
| SF₄Cl | N | C—NO₂ | C—COOEt | C—H | N | TeF₅ | N | C—NO₂ | C—COOEt | C—H | N |
| SF₄Cl | N | C—F | C—NO₂ | C—H | N | TeF₅ | N | C—F | C—NO₂ | C—H | N |
| SF₄Cl | N | C—Cl | C—NO₂ | C—H | N | TeF₅ | N | C—Cl | C—NO₂ | C—H | N |
| SF₄Cl | N | C—Br | C—NO₂ | C—H | N | TeF₅ | N | C—Br | C—NO₂ | C—H | N |
| SF₄Cl | N | C—COOMe | C—NO₂ | C—H | N | TeF₅ | N | C—COOMe | C—NO₂ | C—H | N |
| SF₄Cl | N | C—COOEt | C—NO₂ | C—H | N | TeF₅ | N | C—COOEt | C—NO₂ | C—H | N |
| SF₄Cl | N | C—CF₃ | C—F | C—H | N | TeF₅ | N | C—CF₃ | C—F | C—H | N |
| SF₄Cl | N | C—CF₃ | C—Cl | C—H | N | TeF₅ | N | C—CF₃ | C—Cl | C—H | N |
| SF₄Cl | N | C—CF₃ | C—Br | C—H | N | TeF₅ | N | C—CF₃ | C—Br | C—H | N |
| SF₄Cl | N | C—CF₃ | C—OAc | C—H | N | TeF₅ | N | C—CF₃ | C—OAc | C—H | N |
| SF₄Cl | N | C—CF₃ | C—NO₂ | C—H | N | TeF₅ | N | C—CF₃ | C—NO₂ | C—H | N |
| SF₄Cl | N | C—NPhth | C—H | C—H | N | TeF₅ | N | C—NPhth | C—H | C—H | N |
| SF₄Cl | N | C—H | C—NPhth | C—H | N | TeF₅ | N | C—H | C—NPhth | C—H | N |
| SF₄Cl | N | C—H | C—H | C—NPhth | N | TeF₅ | N | C—H | C—H | C—NPhth | N |
| SF₄Cl | N | C—OBz | C—H | C—H | N | TeF₅ | N | C—OBz | C—H | C—H | N |
| SF₄Cl | N | C—H | C—OBz | C—H | N | TeF₅ | N | C—H | C—OBz | C—H | N |
| SF₄Cl | N | C—H | C—H | C—OBz | N | TeF₅ | N | C—H | C—H | C—OBz | N |
| SF₄Cl | N | C—N₃ | C—H | C—H | N | TeF₅ | N | C—N₃ | C—H | C—H | N |
| SF₄Cl | N | C—H | C—N₃ | C—H | N | TeF₅ | N | C—H | C—N₃ | C—H | N |
| SF₄Cl | N | C—H | C—H | C—N₃ | N | TeF₅ | N | C—H | C—H | C—N₃ | N |
| SF₄Cl | N | N | C—H | C—F | N | TeF₅ | N | N | C—H | C—F | N |
| SF₄Cl | N | N | C—H | C—Cl | N | TeF₅ | N | N | C—H | C—Cl | N |
| SF₄Cl | N | N | C—H | C—Br | N | TeF₅ | N | N | C—H | C—Br | N |
| SF₄Cl | N | N | C—H | C—NO₂ | N | TeF₅ | N | N | C—H | C—NO₂ | N |
| SF₄Cl | N | N | C—H | C—CF₃ | N | TeF₅ | N | N | C—H | C—CF₃ | N |
| SF₄Cl | N | N | C—H | C—COOMe | N | TeF₅ | N | N | C—H | C—COOMe | N |
| SF₄Cl | N | N | C—H | C—COOEt | N | TeF₅ | N | N | C—H | C—COOEt | N |
| SF₄Cl | N | N | C—H | C—OAc | N | TeF₅ | N | N | C—H | C—OAc | N |
| SF₄Cl | N | N | C—H | C—SF₅ | N | TeF₅ | N | N | C—H | C—SF₅ | N |
| SF₄Cl | N | N | C—H | C—tBu | N | TeF₅ | N | N | C—H | C—tBu | N |
| SF₄Cl | N | N | C—H | C—Ph | N | TeF₅ | N | N | C—H | C—Ph | N |
| SF₄Cl | N | N | C—F | C—H | N | TeF₅ | N | N | C—F | C—H | N |
| SF₄Cl | N | N | C—Cl | C—H | N | TeF₅ | N | N | C—Cl | C—H | N |
| SF₄Cl | N | N | C—Br | C—H | N | TeF₅ | N | N | C—Br | C—H | N |
| SF₄Cl | N | N | C—NO₂ | C—H | N | TeF₅ | N | N | C—NO₂ | C—H | N |
| SF₄Cl | N | N | C—CF₃ | C—H | N | TeF₅ | N | N | C—CF₃ | C—H | N |
| SF₄Cl | N | N | C—COOMe | C—H | N | TeF₅ | N | N | C—COOMe | C—H | N |
| SF₄Cl | N | N | C—COOEt | C—H | N | TeF₅ | N | N | C—COOEt | C—H | N |
| SF₄Cl | N | N | C—OAc | C—H | N | TeF₅ | N | N | C—OAc | C—H | N |
| SF₄Cl | N | N | C—SF₅ | C—H | N | TeF₅ | N | N | C—SF₅ | C—H | N |
| SF₄Cl | N | N | C—tBu | C—H | N | TeF₅ | N | N | C—tBu | C—H | N |
| SF₄Cl | N | N | C—Ph | C—H | N | TeF₅ | N | N | C—Ph | C—H | N |
| SF₄Cl | N | N | C—F | C—F | N | TeF₅ | N | N | C—F | C—F | N |
| SF₄Cl | N | N | C—Cl | C—F | N | TeF₅ | N | N | C—Cl | C—F | N |
| SF₄Cl | N | N | C—Br | C—F | N | TeF₅ | N | N | C—Br | C—F | N |
| SF₄Cl | N | N | C—F | C—Cl | N | TeF₅ | N | N | C—F | C—Cl | N |
| SF₄Cl | N | N | C—Cl | C—Cl | N | TeF₅ | N | N | C—Cl | C—Cl | N |
| SF₄Cl | N | N | C—Br | C—Cl | N | TeF₅ | N | N | C—Br | C—Cl | N |
| SF₄Cl | N | N | C—F | C—Br | N | TeF₅ | N | N | C—F | C—Br | N |
| SF₄Cl | N | N | C—Cl | C—Br | N | TeF₅ | N | N | C—Cl | C—Br | N |
| SF₄Cl | N | N | C—Cl | C—Br | N | TeF₅ | N | N | C—Cl | C—Br | N |
| SF₄Cl | N | N | C—F | C—NO₂ | N | TeF₅ | N | N | C—F | C—NO₂ | N |
| SF₄Cl | N | N | C—Cl | C—NO₂ | N | TeF₅ | N | N | C—Cl | C—NO₂ | N |
| SF₄Cl | N | N | C—Br | C—NO₂ | N | TeF₅ | N | N | C—Br | C—NO₂ | N |
| SF₄Cl | N | N | C—COOMe | C—NO₂ | N | TeF₅ | N | N | C—COOMe | C—NO₂ | N |
| SF₄Cl | N | N | C—COOEt | C—NO₂ | N | TeF₅ | N | N | C—COOEt | C—NO₂ | N |
| SF₄Cl | N | N | C—NO₂ | C—F | N | TeF₅ | N | N | C—NO₂ | C—F | N |
| SF₄Cl | N | N | C—NO₂ | C—Cl | N | TeF₅ | N | N | C—NO₂ | C—Cl | N |
| SF₄Cl | N | N | C—NO₂ | C—Br | N | TeF₅ | N | N | C—NO₂ | C—Br | N |
| SF₄Cl | N | N | C—NO₂ | C—COOMe | N | TeF₅ | N | N | C—NO₂ | C—COOMe | N |
| SF₄Cl | N | N | C—NO₂ | C—COOEt | N | TeF₅ | N | N | C—NO₂ | C—COOEt | N |
| SF₄Cl | N | N | C—F | C—CF₃ | N | TeF₅ | N | N | C—F | C—CF₃ | N |
| SF₄Cl | N | N | C—Cl | C—CF₃ | N | TeF₅ | N | N | C—Cl | C—CF₃ | N |
| SF₄Cl | N | N | C—Br | C—CF₃ | N | TeF₅ | N | N | C—Br | C—CF₃ | N |
| SF₄Cl | N | N | C—OAc | C—CF₃ | N | TeF₅ | N | N | C—OAc | C—CF₃ | N |
| SF₄Cl | N | N | C—NPhth | C—H | N | TeF₅ | N | N | C—NPhth | C—H | N |
| SF₄Cl | N | N | C—H | C—NPhth | N | TeF₅ | N | N | C—H | C—NPhth | N |
| SF₄Cl | N | N | C—OBz | C—H | N | TeF₅ | N | N | C—OBz | C—H | N |
| SF₄Cl | N | N | C—H | C—OBz | N | TeF₅ | N | N | C—H | C—OBz | N |
| SF₄Cl | N | N | C—N₃ | C—H | N | TeF₅ | N | N | C—N₃ | C—H | N |
| SF₄Cl | N | N | C—H | C—N₃ | N | TeF₅ | N | N | C—H | C—N₃ | N |
| SF₅ | C—H | C—H | C—H | C—H | C—H | SeF₃ | C—H | C—H | C—H | C—H | C—H |
| SF₅ | C—H | C—H | C—F | C—H | C—H | SeF₃ | C—H | C—H | C—F | C—H | C—H |
| SF₅ | C—H | C—H | C—Cl | C—H | C—H | SeF₃ | C—H | C—H | C—Cl | C—H | C—H |
| SF₅ | C—H | C—H | C—Br | C—H | C—H | SeF₃ | C—H | C—H | C—Br | C—H | C—H |
| SF₅ | C—H | C—H | C—NO₂ | C—H | C—H | SeF₃ | C—H | C—H | C—NO₂ | C—H | C—H |
| SF₅ | C—H | C—H | C—CF₃ | C—H | C—H | SeF₃ | C—H | C—H | C—CF₃ | C—H | C—H |
| SF₅ | C—H | C—H | C—COOMe | C—H | C—H | SeF₃ | C—H | C—H | C—COCMe | C—H | C—H |
| SF₅ | C—H | C—H | C—COOEt | C—H | C—H | SeF₃ | C—H | C—H | C—COOEt | C—H | C—H |
| SF₅ | C—H | C—H | C—OAc | C—H | C—H | SeF₃ | C—H | C—H | C—OAc | C—H | C—H |
| SF₅ | C—H | C—H | C—SF₅ | C—H | C—H | SeF₃ | C—H | C—H | C—SF₅ | C—H | C—H |
| SF₅ | C—H | C—H | C—tBu | C—H | C—H | SeF₃ | C—H | C—H | C—tBu | C—H | C—H |
| SF₅ | C—H | C—H | C—Ph | C—H | C—H | SeF₃ | C—H | C—H | C—Ph | C—H | C—H |
| SF₅ | C—H | C—NPhth | C—H | C—H | C—H | SeF₃ | C—H | C—NPhth | C—H | C—H | C—H |
| SF₅ | C—H | C—H | C—NPhth | C—H | C—H | SeF₃ | C—H | C—H | C—NPhth | C—H | C—H |
| SF₅ | C—H | C—OBz | C—H | C—H | C—H | SeF₃ | C—H | C—OBz | C—H | C—H | C—H |
| SF₅ | C—H | C—H | C—OBz | C—H | C—H | SeF₃ | C—H | C—H | C—OBz | C—H | C—H |
| SF₅ | C—H | C—N₃ | C—H | C—H | C—H | SeF₃ | C—H | C—N₃ | C—H | C—H | C—H |
| SF₅ | C—H | C—H | C—N₃ | C—H | C—H | SeF₃ | C—H | C—H | C—N₃ | C—H | C—H |
| SF₅ | C—F | C—H | C—H | C—H | C—H | SeF₃ | C—F | C—H | C—H | C—H | C—H |
| SF₅ | C—F | C—H | C—F | C—H | C—H | SeF₃ | C—F | C—H | C—F | C—H | C—H |
| SF₅ | C—F | C—H | C—Cl | C—H | C—H | SeF₃ | C—F | C—H | C—Cl | C—H | C—H |
| SF₅ | C—F | C—H | C—Br | C—H | C—H | SeF₃ | C—F | C—H | C—Br | C—H | C—H |
| SF₅ | C—F | C—H | C—NO₂ | C—H | C—H | SeF₃ | C—F | C—H | C—NO₂ | C—H | C—H |
| SF₅ | C—F | C—H | C—CF₃ | C—H | C—H | SeF₃ | C—F | C—H | C—CF₃ | C—H | C—H |
| SF₅ | C—F | C—H | C—COOMe | C—H | C—H | SeF₃ | C—F | C—H | C—COOMe | C—H | C—H |
| SF₅ | C—F | C—H | C—COOEt | C—H | C—H | SeF₃ | C—F | C—H | C—COOEt | C—H | C—H |
| SF₅ | C—F | C—H | C—OAc | C—H | C—H | SeF₃ | C—F | C—H | C—OAc | C—H | C—H |
| SF₅ | C—F | C—H | C—SF₅ | C—H | C—H | SeF₃ | C—F | C—H | C—SF₅ | C—H | C—H |
| SF₅ | C—F | C—H | C—tBu | C—H | C—H | SeF₃ | C—F | C—H | C—tBu | C—H | C—H |
| SF₅ | C—F | C—H | C—Ph | C—H | C—H | SeF₃ | C—F | C—H | C—Ph | C—H | C—H |
| SF₅ | C—F | C—NPhth | C—H | C—H | C—H | SeF₃ | C—F | C—NPhth | C—H | C—H | C—H |
| SF₅ | C—F | C—H | C—NPhth | C—H | C—H | SeF₃ | C—F | C—H | C—NPhth | C—H | C—H |
| SF₅ | C—F | C—H | C—H | C—NPhth | C—H | SeF₃ | C—F | C—H | C—H | C—NPhth | C—H |
| SF₅ | C—F | C—OBz | C—H | C—H | C—H | SeF₃ | C—F | C—OBz | C—H | C—H | C—H |
| SF₅ | C—F | C—H | C—OBz | C—H | C—H | SeF₃ | C—F | C—H | C—OBz | C—H | C—H |
| SF₅ | C—F | C—N₃ | C—H | C—H | C—H | SeF₃ | C—F | C—N₃ | C—H | C—H | C—H |
| SF₅ | C—F | C—H | C—N₃ | C—H | C—H | SeF₃ | C—F | C—H | C—N₃ | C—H | C—H |
| SF₅ | C—H | C—F | C—F | C—H | C—H | SeF₃ | C—H | C—F | C—F | C—H | C—H |
| SF₅ | C—H | C—F | C—Cl | C—H | C—H | SeF₃ | C—H | C—F | C—Cl | C—H | C—H |
| SF₅ | C—H | C—F | C—Br | C—H | C—H | SeF₃ | C—H | C—F | C—Br | C—H | C—H |
| SF₅ | C—H | C—Cl | C—F | C—H | C—H | SeF₃ | C—H | C—Cl | C—F | C—H | C—H |
| SF₅ | C—H | C—Cl | C—Cl | C—H | C—H | SeF₃ | C—H | C—Cl | C—Cl | C—H | C—H |
| SF₅ | C—H | C—Cl | C—Br | C—H | C—H | SeF₃ | C—H | C—Cl | C—Br | C—H | C—H |
| SF₅ | C—H | C—Br | C—F | C—H | C—H | SeF₃ | C—H | C—Br | C—F | C—H | C—H |
| SF₅ | C—H | C—Br | C—Cl | C—H | C—H | SeF₃ | C—H | C—Br | C—Cl | C—H | C—H |
| SF₅ | C—H | C—Br | C—Cl | C—H | C—H | SeF₃ | C—H | C—Br | C—Cl | C—H | C—H |
| SF₅ | C—H | C—NO₂ | C—F | C—H | C—H | SeF₃ | C—H | C—NO₂ | C—F | C—H | C—H |
| SF₅ | C—H | C—NO₂ | C—Cl | C—H | C—H | SeF₃ | C—H | C—NO₂ | C—Cl | C—H | C—H |
| SF₅ | C—H | C—NO₂ | C—Br | C—H | C—H | SeF₃ | C—H | C—NO₂ | C—Br | C—H | C—H |
| SF₅ | C—H | C—NO₂ | C—COOMe | C—H | C—H | SeF₃ | C—H | C—NO₂ | C—COOMe | C—H | C—H |
| SF₅ | C—H | C—NO₂ | C—COOEt | C—H | C—H | SeF₃ | C—H | C—NO₂ | C—COOEt | C—H | C—H |
| SF₅ | C—H | C—F | C—NO₂ | C—H | C—H | SeF₃ | C—H | C—F | C—NO₂ | C—H | C—H |
| SF₅ | C—H | C—Cl | C—NO₂ | C—H | C—H | SeF₃ | C—H | C—Cl | C—NO₂ | C—H | C—H |
| SF₅ | C—H | C—Br | C—NO₂ | C—H | C—H | SeF₃ | C—H | C—Br | C—NO₂ | C—H | C—H |
| SF₅ | C—H | C—COOMe | C—NO₂ | C—H | C—H | SeF₃ | C—H | C—COOMe | C—NO₂ | C—H | C—H |
| SF₅ | C—H | C—COOEt | C—NO₂ | C—H | C—H | SeF₃ | C—H | C—COOEt | C—NO₂ | C—H | C—H |
| SF₅ | C—H | C—CF₃ | C—F | C—H | C—H | SeF₃ | C—H | C—CF₃ | C—F | C—H | C—H |
| SF₅ | C—H | C—CF₃ | C—Cl | C—H | C—H | SeF₃ | C—H | C—CF₃ | C—Cl | C—H | C—H |
| SF₅ | C—H | C—CF₃ | C—Br | C—H | C—H | SeF₃ | C—H | C—CF₃ | C—Br | C—H | C—H |
| SF₅ | C—H | C—CF₃ | C—OAc | C—H | C—H | SeF₃ | C—H | C—CF₃ | C—OAc | C—H | C—H |
| SF₅ | C—H | C—CF₃ | C—NO₂ | C—H | C—H | SeF₃ | C—H | C—CF₃ | C—NO₂ | C—H | C—H |
| SF₅ | C—F | C—F | C—CF₃ | C—H | C—H | SeF₃ | C—F | C—F | C—CF₃ | C—H | C—H |
| SF₅ | C—F | C—Cl | C—CF₃ | C—H | C—H | SeF₃ | C—F | C—Cl | C—CF₃ | C—H | C—H |
| SF₅ | C—F | C—Br | C—CF₃ | C—H | C—H | SeF₃ | C—F | C—Br | C—CF₃ | C—H | C—H |
| SF₅ | C—F | C—OAc | C—CF₃ | C—H | C—H | SeF₃ | C—F | C—OAc | C—CF₃ | C—H | C—H |
| SF₅ | C—F | C—NO₂ | C—CF₃ | C—H | C—H | SeF₃ | C—F | C—NO₂ | C—CF₃ | C—H | C—H |
| SF₅ | C—F | C—F | C—F | C—H | C—H | SeF₃ | C—F | C—F | C—F | C—H | C—H |
| SF₅ | C—F | C—F | C—Cl | C—H | C—H | SeF₃ | C—F | C—F | C—Cl | C—H | C—H |
| SF₅ | C—F | C—F | C—Br | C—H | C—H | SeF₃ | C—F | C—F | C—Br | C—H | C—H |
| SF₅ | C—F | C—Cl | C—F | C—H | C—H | SeF₃ | C—F | C—Cl | C—F | C—H | C—H |
| SF₅ | C—F | C—Cl | C—Cl | C—H | C—H | SeF₃ | C—F | C—Cl | C—Cl | C—H | C—H |
| SF₅ | C—F | C—Cl | C—Br | C—H | C—H | SeF₃ | C—F | C—Cl | C—Br | C—H | C—H |
| SF₅ | C—F | C—Br | C—F | C—H | C—H | SeF₃ | C—F | C—Br | C—F | C—H | C—H |
| SF₅ | C—F | C—Br | C—Cl | C—H | C—H | SeF₃ | C—F | C—Br | C—Cl | C—H | C—H |
| SF₅ | C—F | C—Br | C—Cl | C—H | C—H | SeF₃ | C—F | C—Br | C—Cl | C—H | C—H |
| SF₅ | C—F | C—NO₂ | C—F | C—H | C—H | SeF₃ | C—F | C—NO₂ | C—F | C—H | C—H |
| SF₅ | C—F | C—NO₂ | C—Cl | C—H | C—H | SeF₃ | C—F | C—NO₂ | C—Cl | C—H | C—H |
| SF₅ | C—F | C—NO₂ | C—Br | C—H | C—H | SeF₃ | C—F | C—NO₂ | C—Br | C—H | C—H |
| SF₅ | C—F | C—NO₂ | C—COOMe | C—H | C—H | SeF₃ | C—F | C—NO₂ | C—COOMe | C—H | C—H |
| SF₅ | C—F | C—NO₂ | C—COOEt | C—H | C—H | SeF₃ | C—F | C—NO₂ | C—COOEt | C—H | C—H |
| SF₅ | C—F | C—F | C—NO₂ | C—H | C—H | SeF₃ | C—F | C—F | C—NO₂ | C—H | C—H |
| SF₅ | C—F | C—Cl | C—NO₂ | C—H | C—H | SeF₃ | C—F | C—Cl | C—NO₂ | C—H | C—H |
| SF₅ | C—F | C—Br | C—NO₂ | C—H | C—H | SeF₃ | C—F | C—Br | C—NO₂ | C—H | C—H |
| SF₅ | C—F | C—COOMe | C—NO₂ | C—H | C—H | SeF₃ | C—F | C—COOMe | C—NO₂ | C—H | C—H |
| SF₅ | C—F | C—COOEt | C—NO₂ | C—H | C—H | SeF₃ | C—F | C—COOEt | C—NO₂ | C—H | C—H |
| SF₅ | C—F | C—CF₃ | C—F | C—H | C—H | SeF₃ | C—F | C—CF₃ | C—F | C—H | C—H |
| SF₅ | C—F | C—CF₃ | C—Cl | C—H | C—H | SeF₃ | C—F | C—CF₃ | C—Cl | C—H | C—H |
| SF₅ | C—F | C—CF₃ | C—Br | C—H | C—H | SeF₃ | C—F | C—CF₃ | C—Br | C—H | C—H |
| SF₅ | C—F | C—CF₃ | C—OAc | C—H | C—H | SeF₃ | C—F | C—CF₃ | C—OAc | C—H | C—H |
| SF₅ | C—F | C—CF₃ | C—NO₂ | C—H | C—H | SeF₃ | C—F | C—CF₃ | C—NO₂ | C—H | C—H |
| SF₅ | C—F | C—F | C—CF₃ | C—H | C—H | SeF₃ | C—F | C—F | C—CF₃ | C—H | C—H |
| SF₅ | C—F | C—Cl | C—CF₃ | C—H | C—H | SeF₃ | C—F | C—Cl | C—CF₃ | C—H | C—H |
| SF₅ | C—F | C—Br | C—CF₃ | C—H | C—H | SeF₃ | C—F | C—Br | C—CF₃ | C—H | C—H |
| SF₅ | C—F | C—OAc | C—CF₃ | C—H | C—H | SeF₃ | C—F | C—OAc | C—CF₃ | C—H | C—H |
| SF₅ | C—F | C—NO₂ | C—CF₃ | C—H | C—H | SeF₃ | C—F | C—NO₂ | C—CF₃ | C—H | C—H |
| SF₅ | N | C—F | C—H | C—H | C—H | SeF₃ | N | C—F | C—H | C—H | C—H |
| SF₅ | N | C—Cl | C—H | C—H | C—H | SeF₃ | N | C—Cl | C—H | C—H | C—H |
| SF₅ | N | C—Br | C—H | C—H | C—H | SeF₃ | N | C—Br | C—H | C—H | C—H |
| SF₅ | N | C—NO₂ | C—H | C—H | C—H | SeF₃ | N | C—NO₂ | C—H | C—H | C—H |
| SF₅ | N | C—CF₃ | C—H | C—H | C—H | SeF₃ | N | C—CF₃ | C—H | C—H | C—H |
| SF₅ | N | C—COOMe | C—H | C—H | C—H | SeF₃ | N | C—COOMe | C—H | C—H | C—H |
| SF₅ | N | C—COOEt | C—H | C—H | C—H | SeF₃ | N | C—COOEt | C—H | C—H | C—H |
| SF₅ | N | C—OAc | C—H | C—H | C—H | SeF₃ | N | C—OAc | C—H | C—H | C—H |
| SF₅ | N | C—SF₅ | C—H | C—H | C—H | SeF₃ | N | C—SF₅ | C—H | C—H | C—H |
| SF₅ | N | C—tBu | C—H | C—H | C—H | SeF₃ | N | C—tBu | C—H | C—H | C—H |
| SF₅ | N | C—Ph | C—H | C—H | C—H | SeF₃ | N | C—Ph | C—H | C—H | C—H |
| SF₅ | N | C—H | C—H | C—H | C—H | SeF₃ | N | C—H | C—H | C—H | C—H |
| SF₅ | N | C—H | C—H | C—H | C—H | SeF₃ | N | C—H | C—H | C—H | C—H |
| SF₅ | N | C—H | C—F | C—H | C—H | SeF₃ | N | C—H | C—F | C—H | C—H |
| SF₅ | N | C—H | C—Cl | C—H | C—H | SeF₃ | N | C—H | C—Cl | C—H | C—H |
| SF₅ | N | C—H | C—Br | C—H | C—H | SeF₃ | N | C—H | C—Br | C—H | C—H |
| SF₅ | N | C—H | C—NO₂ | C—H | C—H | SeF₃ | N | C—H | C—NO₂ | C—H | C—H |
| SF₅ | N | C—H | C—CF₃ | C—H | C—H | SeF₃ | N | C—H | C—CF₃ | C—H | C—H |
| SF₅ | N | C—H | C—COOMe | C—H | C—H | SeF₃ | N | C—H | C—COOMe | C—H | C—H |
| SF₅ | N | C—H | C—COOEt | C—H | C—H | SeF₃ | N | C—H | C—COOEt | C—H | C—H |
| SF₅ | N | C—H | C—OAc | C—H | C—H | SeF₃ | N | C—H | C—OAc | C—H | C—H |
| SF₅ | N | C—H | C—SF6 | C—H | C—H | SeF₃ | N | C—H | C—SF₅ | C—H | C—H |
| SF₅ | N | C—H | C—tBu | C—H | C—H | SeF₃ | N | C—H | C—tBu | C—H | C—H |
| SF₅ | N | C—H | C—Ph | C—H | C—H | SeF₃ | N | C—H | C—Ph | C—H | C—H |
| SF₅ | N | C—F | C—F | C—H | C—H | SeF₃ | N | C—F | C—F | C—H | C—H |
| SF₅ | N | C—F | C—Cl | C—H | C—H | SeF₃ | N | C—F | C—Cl | C—H | C—H |
| SF₅ | N | C—F | C—Br | C—H | C—H | SeF₃ | N | C—F | C—Br | C—H | C—H |
| SF₅ | N | C—Cl | C—F | C—H | C—H | SeF₃ | N | C—Cl | C—F | C—H | C—H |
| SF₅ | N | C—Cl | C—Cl | C—H | C—H | SeF₃ | N | C—Cl | C—Cl | C—H | C—H |
| SF₅ | N | C—Cl | C—Br | C—H | C—H | SeF₃ | N | C—Cl | C—Br | C—H | C—H |
| SF₅ | N | C—Br | C—F | C—H | C—H | SeF₃ | N | C—Br | C—F | C—H | C—H |
| SF₅ | N | C—Br | C—Cl | C—H | C—H | SeF₃ | N | C—Br | C—Cl | C—H | C—H |
| SF₅ | N | C—Br | C—Cl | C—H | C—H | SeF₃ | N | C—Br | C—Cl | C—H | C—H |
| SF₅ | N | C—NO₂ | C—F | C—H | C—H | SeF₃ | N | C—NO₂ | C—F | C—H | C—H |
| SF₅ | N | C—NO₂ | C—Cl | C—H | C—H | SeF₃ | N | C—NO₂ | C—Cl | C—H | C—H |
| SF₅ | N | C—NO₂ | C—Br | C—H | C—H | SeF₃ | N | C—NO₂ | C—Br | C—H | C—H |
| SF₅ | N | C—NO₂ | C—COCMe | C—H | C—H | SeF₃ | N | C—NO₂ | C—COOMe | C—H | C—H |
| SF₅ | N | C—NO₂ | C—COOEt | C—H | C—H | SeF₃ | N | C—NO₂ | C—COOEt | C—H | C—H |
| SF₅ | N | C—F | C—NO₂ | C—H | C—H | SeF₃ | N | C—F | C—NO₂ | C—H | C—H |
| SF₅ | N | C—Cl | C—NO₂ | C—H | C—H | SeF₃ | N | C—Cl | C—NO₂ | C—H | C—H |
| SF₅ | N | C—Br | C—NO₂ | C—H | C—H | SeF₃ | N | C—Br | C—NO₂ | C—H | C—H |
| SF₅ | N | C—COOMe | C—NO₂ | C—H | C—H | SeF₃ | N | C—COOMe | C—NO₂ | C—H | C—H |
| SF₅ | N | C—COOEt | C—NO₂ | C—H | C—H | SeF₃ | N | C—COOEt | C—NO₂ | C—H | C—H |
| SF₅ | N | C—CF₃ | C—F | C—H | C—H | SeF₃ | N | C—CF₃ | C—F | C—H | C—H |
| SF₅ | N | C—CF₃ | C—Cl | C—H | C—H | SeF₃ | N | C—CF₃ | C—Cl | C—H | C—H |
| SF₅ | N | C—CF₃ | C—Br | C—H | C—H | SeF₃ | N | C—CF₃ | C—Br | C—H | C—H |
| SF₅ | N | C—CF₃ | C—OAc | C—H | C—H | SeF₃ | N | C—CF₃ | C—OAc | C—H | C—H |
| SF₅ | N | C—CF₃ | C—NO₂ | C—H | C—H | SeF₃ | N | C—CF₃ | C—NO₂ | C—H | C—H |
| SF₅ | N | C—NPhth | C—H | C—H | C—H | SeF₃ | N | C—NPhth | C—H | C—H | C—H |
| SF₅ | N | C—H | C—NPhth | C—H | C—H | SeF₃ | N | C—H | C—NPhth | C—H | C—H |
| SF₅ | N | C—H | C—H | C—NPhth | C—H | SeF₃ | N | C—H | C—H | C—NPhth | C—H |
| SF₅ | N | C—OBz | C—H | C—H | C—H | SeF₃ | N | C—OBz | C—H | C—H | C—H |
| SF₅ | N | C—H | C—OBz | C—H | C—H | SeF₃ | N | C—H | C—OBz | C—H | C—H |
| SF₅ | N | C—N₃ | C—H | C—H | C—H | SeF₃ | N | C—N₃ | C—H | C—H | C—H |
| SF₅ | N | C—H | C—N₃ | C—H | C—H | SeF₃ | N | C—H | C—N₃ | C—H | C—H |
| SF₅ | N | C—F | C—H | C—H | N | SeF₃ | N | C—F | C—H | C—H | N |
| SF₅ | N | C—Cl | C—H | C—H | N | SeF₃ | N | C—Cl | C—H | C—H | N |
| SF₅ | N | C—Br | C—H | C—H | N | SeF₃ | N | C—Br | C—H | C—H | N |
| SF₅ | N | C—NO₂ | C—H | C—H | N | SeF₃ | N | C—NO₂ | C—H | C—H | N |
| SF₅ | N | C—CF₃ | C—H | C—H | N | SeF₃ | N | C—CF₃ | C—H | C—H | N |
| SF₅ | N | C—COOMe | C—H | C—H | N | SeF₃ | N | C—COOMe | C—H | C—H | N |
| SF₅ | N | C—COOEt | C—H | C—H | N | SeF₃ | N | C—COOEt | C—H | C—H | N |
| SF₅ | N | C—OAc | C—H | C—H | N | SeF₃ | N | C—OAc | C—H | C—H | N |
| SF₅ | N | C—SF₅ | C—H | C—H | N | SeF₃ | N | C—SF₅ | C—H | C—H | N |
| SF₅ | N | C—tBu | C—H | C—H | N | SeF₃ | N | C—tBu | C—H | C—H | N |
| SF₅ | N | C—Ph | C—H | C—H | N | SeF₃ | N | C—Ph | C—H | C—H | N |
| SF₅ | N | C—H | C—F | C—H | N | SeF₃ | N | C—H | C—F | C—H | N |
| SF₅ | N | C—H | C—Cl | C—H | N | SeF₃ | N | C—H | C—Cl | C—H | N |
| SF₅ | N | C—H | C—Br | C—H | N | SeF₃ | N | C—H | C—Br | C—H | N |
| SF₅ | N | C—H | C—NO₂ | C—H | N | SeF₃ | N | C—H | C—NO₂ | C—H | N |
| SF₅ | N | C—H | C—CF₃ | C—H | N | SeF₃ | N | C—H | C—CF₃ | C—H | N |
| SF₅ | N | C—H | C—COOMe | C—H | N | SeF₃ | N | C—H | C—COOMe | C—H | N |
| SF₅ | N | C—H | C—COOEt | C—H | N | SeF₃ | N | C—H | C—COOEt | C—H | N |
| SF₅ | N | C—H | C—OAc | C—H | N | SeF₃ | N | C—H | C—OAc | C—H | N |
| SF₅ | N | C—H | C—SF₅ | C—H | N | SeF₃ | N | C—H | C—SF₅ | C—H | N |
| SF₅ | N | C—H | C—tBu | C—H | N | SeF₃ | N | C—H | C—tBu | C—H | N |
| SF₅ | N | C—H | C—Ph | C—H | N | SeF₃ | N | C—H | C—Ph | C—H | N |
| SF₅ | N | C—F | C—F | C—H | N | SeF₃ | N | C—F | C—F | C—H | N |
| SF₅ | N | C—F | C—Cl | C—H | N | SeF₃ | N | C—F | C—Cl | C—H | N |
| SF₅ | N | C—F | C—Br | C—H | N | SeF₃ | N | C—F | C—Br | C—H | N |
| SF₅ | N | C—Cl | C—F | C—H | N | SeF₃ | N | C—Cl | C—F | C—H | N |
| SF₅ | N | C—Cl | C—Cl | C—H | N | SeF₃ | N | C—Cl | C—Cl | C—H | N |
| SF₅ | N | C—Cl | C—Br | C—H | N | SeF₃ | N | C—Cl | C—Br | C—H | N |
| SF₅ | N | C—Br | C—F | C—H | N | SeF₃ | N | C—Br | C—F | C—H | N |
| SF₅ | N | C—Br | C—Cl | C—H | N | SeF₃ | N | C—Br | C—Cl | C—H | N |
| SF₅ | N | C—Br | C—Cl | C—H | N | SeF₃ | N | C—Br | C—Cl | C—H | N |
| SF₅ | N | C—NO₂ | C—F | C—H | N | SeF₃ | N | C—NO₂ | C—F | C—H | N |
| SF₅ | N | C—NO₂ | C—Cl | C—H | N | SeF₃ | N | C—NO₂ | C—Cl | C—H | N |
| SF₅ | N | C—NO₂ | C—Br | C—H | N | SeF₃ | N | C—NO₂ | C—Br | C—H | N |
| SF₅ | N | C—NO₂ | C—COOMe | C—H | N | SeF₃ | N | C—NO₂ | C—COOMe | C—H | N |
| SF₅ | N | C—NO₂ | C—COOEt | C—H | N | SeF₃ | N | C—NO₂ | C—COOEt | C—H | N |
| SF₅ | N | C—F | C—NO₂ | C—H | N | SeF₃ | N | C—F | C—NO₂ | C—H | N |
| SF₅ | N | C—Cl | C—NO₂ | C—H | N | SeF₃ | N | C—Cl | C—NO₂ | C—H | N |
| SF₅ | N | C—Br | C—NO₂ | C—H | N | SeF₃ | N | C—Br | C—NO₂ | C—H | N |
| SF₅ | N | C—COOMe | C—NO₂ | C—H | N | SeF₃ | N | C—COOMe | C—NO₂ | C—H | N |
| SF₅ | N | C—COOEt | C—NO₂ | C—H | N | SeF₃ | N | C—COOEt | C—NO₂ | C—H | N |
| SF₅ | N | C—CF₃ | C—F | C—H | N | SeF₃ | N | C—CF₃ | C—F | C—H | N |
| SF₅ | N | C—CF₃ | C—Cl | C—H | N | SeF₃ | N | C—CF₃ | C—Cl | C—H | N |
| SF₅ | N | C—CF₃ | C—Br | C—H | N | SeF₃ | N | C—CF₃ | C—Br | C—H | N |
| SF₅ | N | C—CF₃ | C—OAc | C—H | N | SeF₃ | N | C—CF₃ | C—OAc | C—H | N |
| SF₅ | N | C—CF₃ | C—NO₂ | C—H | N | SeF₃ | N | C—CF₃ | C—NO₂ | C—H | N |
| SF₅ | N | C—NPhth | C—H | C—H | N | SeF₃ | N | C—NPhth | C—H | C—H | N |
| SF₅ | N | C—H | C—NPhth | C—H | N | SeF₃ | N | C—H | C—NPhth | C—H | N |
| SF₅ | N | C—H | C—H | C—NPhth | N | SeF₃ | N | C—H | C—H | C—NPhth | N |
| SF₅ | N | C—OBz | C—H | C—H | N | SeF₃ | N | C—OBz | C—H | C—H | N |
| SF₅ | N | C—H | C—OBz | C—H | N | SeF₃ | N | C—H | C—OBz | C—H | N |
| SF₅ | N | C—N₃ | C—H | C—H | N | SeF₃ | N | C—N₃ | C—H | C—H | N |
| SF₅ | N | C—H | C—N₃ | C—H | N | SeF₃ | N | C—H | C—N₃ | C—H | N |
| SF₅ | N | N | C—H | C—F | N | SeF₃ | N | N | C—H | C—F | N |
| SF₅ | N | N | C—H | C—Cl | N | SeF₃ | N | N | C—H | C—Cl | N |
| SF₅ | N | N | C—H | C—Br | N | SeF₃ | N | N | C—H | C—Br | N |
| SF₅ | N | N | C—H | C—NO₂ | N | SeF₃ | N | N | C—H | C—NO₂ | N |
| SF₅ | N | N | C—H | C—CF₃ | N | SeF₃ | N | N | C—H | C—CF₃ | N |
| SF₅ | N | N | C—H | C—COOMe | N | SeF₃ | N | N | C—H | C—COOMe | N |
| SF₅ | N | N | C—H | C—COOEt | N | SeF₃ | N | N | C—H | C—COOEt | N |
| SF₅ | N | N | C—H | C—OAc | N | SeF₃ | N | N | C—H | C—OAc | N |
| SF₅ | N | N | C—H | C—SF₅ | N | SeF₃ | N | N | C—H | C—SF₅ | N |
| SF₅ | N | N | C—H | C—tBu | N | SeF₃ | N | N | C—H | C—tBu | N |
| SF₅ | N | N | C—H | C—Ph | N | SeF₃ | N | N | C—H | C—Ph | N |
| SF₅ | N | N | C—F | C—H | N | SeF₃ | N | N | C—F | C—H | N |
| SF₅ | N | N | C—Cl | C—H | N | SeF₃ | N | N | C—Cl | C—H | N |
| SF₅ | N | N | C—Br | C—H | N | SeF₃ | N | N | C—Br | C—H | N |
| SF₅ | N | N | C—NO₂ | C—H | N | SeF₃ | N | N | C—NO₂ | C—H | N |
| SF₅ | N | N | C—CF₃ | C—H | N | SeF₃ | N | N | C—CF₃ | C—H | N |
| SF₅ | N | N | C—COOMe | C—H | N | SeF₃ | N | N | C—COOMe | C—H | N |
| SF₅ | N | N | C—COOEt | C—H | N | SeF₃ | N | N | C—COOEt | C—H | N |
| SF₅ | N | N | C—OAc | C—H | N | SeF₃ | N | N | C—OAc | C—H | N |
| SF₅ | N | N | C—SF₅ | C—H | N | SeF₃ | N | N | C—SF₅ | C—H | N |
| SF₅ | N | N | C—tBu | C—H | N | SeF₃ | N | N | C—tBu | C—H | N |
| SF₅ | N | N | C—Ph | C—H | N | SeF₃ | N | N | C—Ph | C—H | N |
| SF₅ | N | N | C—F | C—F | N | SeF₃ | N | N | C—F | C—F | N |
| SF₅ | N | N | C—Cl | C—F | N | SeF₃ | N | N | C—Cl | C—F | N |
| SF₅ | N | N | C—Br | C—F | N | SeF₃ | N | N | C—Br | C—F | N |
| SF₅ | N | N | C—F | C—Cl | N | SeF₃ | N | N | C—F | C—Cl | N |
| SF₅ | N | N | C—Cl | C—Cl | N | SeF₃ | N | N | C—Cl | C—Cl | N |
| SF₅ | N | N | C—Br | C—Cl | N | SeF₃ | N | N | C—Br | C—Cl | N |
| SF₅ | N | N | C—F | C—Br | N | SeF₃ | N | N | C—F | C—Br | N |
| SF₅ | N | N | C—Cl | C—Br | N | SeF₃ | N | N | C—Cl | C—Br | N |
| SF₅ | N | N | C—Cl | C—Br | N | SeF₃ | N | N | C—Cl | C—Br | N |
| SF₅ | N | N | C—F | C—NO₂ | N | SeF₃ | N | N | C—F | C—NO₂ | N |
| SF₅ | N | N | C—Cl | C—NO₂ | N | SeF₃ | N | N | C—Cl | C—NO₂ | N |
| SF₅ | N | N | C—Br | C—NO₂ | N | SeF₃ | N | N | C—Br | C—NO₂ | N |
| SF₅ | N | N | C—COOMe | C—NO₂ | N | SeF₃ | N | N | C—COOMe | C—NO₂ | N |
| SF₅ | N | N | C—COOEt | C—NO₂ | N | SeF₃ | N | N | C—COOEt | C—NO₂ | N |
| SF₅ | N | N | C—NO₂ | C—F | N | SeF₃ | N | N | C—NO₂ | C—F | N |
| SF₅ | N | N | C—NO₂ | C—Cl | N | SeF₃ | N | N | C—NO₂ | C—Cl | N |
| SF₅ | N | N | C—NO₂ | C—Br | N | SeF₃ | N | N | C—NO₂ | C—Br | N |
| SF₅ | N | N | C—NO₂ | C—COOMe | N | SeF₃ | N | N | C—NO₂ | C—COOMe | N |
| SF₅ | N | N | C—NO₂ | C—COOEt | N | SeF₃ | N | N | C—NO₂ | C—COOEt | N |
| SF₅ | N | N | C—F | C—CF₃ | N | SeF₃ | N | N | C—F | C—CF₃ | N |
| SF₅ | N | N | C—Cl | C—CF₃ | N | SeF₃ | N | N | C—Cl | C—CF₃ | N |
| SF₅ | N | N | C—Br | C—CF₃ | N | SeF₃ | N | N | C—Br | C—CF₃ | N |
| SF₅ | N | N | C—OAc | C—CF₃ | N | SeF₃ | N | N | C—OAc | C—CF₃ | N |
| SF₅ | N | N | C—NO₂ | C—CF₃ | N | SeF₃ | N | N | C—NO₂ | C—CF₃ | N |
| SF₅ | N | N | C—NPhth | C—H | N | SeF₃ | N | N | C—NPhth | C—H | N |
| SF₅ | N | N | C—H | C—NPhth | N | SeF₃ | N | N | C—H | C—NPhth | N |
| SF₅ | N | N | C—OBz | C—H | N | SeF₃ | N | N | C—OBz | C—H | N |
| SF₅ | N | N | C—N₃ | C—H | N | SeF₃ | N | N | C—N₃ | C—H | N |

The compounds obtained by the method according to the present invention may be used as synthetic building blocks, pharmaceuticals, materials, reagents, and agrochemicals.

Another aspect of the present invention relates to the following new compounds of formula (I)

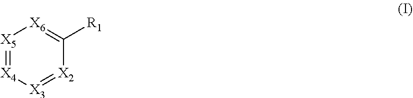

said compounds being selected from the group consisting of

| Compound | | | | | | | Preferred use of |
|---|---|---|---|---|---|---|---|
| No. | R₁ | X₂ | X₃ | X₄ | X₅ | X₆ | the compound |
| 101 | SF₄Cl | C—H | C—H | C—NO₂ | C—COOMe | C—H | Building block for |
| | | | | | | | SF₅— and SF₄R— |
| | | | | | | | containing |
| | | | | | | | compounds. |
| 102 | SF₄Cl | C—H | C—H | C—H | C—COOEt | C—H | Building block for |
| | | | | | | | SF₅— and SF₄R— |
| | | | | | | | containing |
| | | | | | | | compounds. |
| 103 | SF₄Cl | C—H | C—H | C—OAc | C—H | C—H | Building block for |
| | | | | | | | SF₅— and SF₄R— |
| | | | | | | | containing |
| | | | | | | | compounds. |
| 104 | SF₄Cl | C—H | C—H | C—NPhth | C—H | C—H | Building block for |
| | | | | | | | SF₅— and SF₄R— |
| | | | | | | | containing |
| | | | | | | | compounds. |
| 105 | SF₄Cl | C—H | C—H | C—OCF₃ | C—H | C—H | Building block for |
| | | | | | | | SF₅— and SF₄R— |
| | | | | | | | containing |
| | | | | | | | compounds. |
| 106 | SF₄Cl | C—H | C—H | C—SF₅ | C—H | C—H | Building block for |
| | | | | | | | SF₅— and SF₄R— |
| | | | | | | | containing |
| | | | | | | | compounds. |
| 107 | SF₄Cl | N | C—H | C—COOMe | C—H | C—H | Building block for |
| | | | | | | | SF₅— and SF₄R— |
| | | | | | | | containing |
| | | | | | | | compounds. |
| 108 | SF₄Cl | N | N | C—Ph | C—Ph | N | Building block for |
| | | | | | | | SF₅— and SF₄R— |
| | | | | | | | containing |
| | | | | | | | compounds. |
| 109 | SF₅ | C—H | C—H | C—NO₂ | C—COOMe | C—H | Possible |
| | | | | | | | applications in the |
| | | | | | | | synthesis of |
| | | | | | | | pharmaceuticals, |
| | | | | | | | agrochemicals, |
| | | | | | | | and/or liquid |
| | | | | | | | crystals. |
| 111 | SF₅ | C—H | C—H | C—OAc | C—H | C—H | Possible |
| | | | | | | | applications in the |
| | | | | | | | synthesis of |
| | | | | | | | pharmaceuticals, |
| | | | | | | | agrochemicals, |
| | | | | | | | and/or liquid |
| | | | | | | | crystals. |
| 112 | SF₅ | C—H | C—H | C—NO₂ | C—COOH | C—H | Possible |
| | | | | | | | applications in the |
| | | | | | | | synthesis of |
| | | | | | | | pharmaceuticals, |
| | | | | | | | agrochemicals, |
| | | | | | | | and/or liquid |
| | | | | | | | crystals. |
| 116 | SF₅ | C—H | C—H | C—OCF₃ | C—H | C—H | Possible |
| | | | | | | | applications in the |
| | | | | | | | synthesis of |
| | | | | | | | pharmaceuticals, |
| | | | | | | | agrochemicals, |
| | | | | | | | and/or liquid |
| | | | | | | | crystals. |
| 118 | SF₅ | N | C—H | C—COOMe | C—H | C—H | Possible |
| | | | | | | | applications in the |
| | | | | | | | synthesis of |
| | | | | | | | pharmaceuticals, |
| | | | | | | | agrochemicals, |
| | | | | | | | and/or liquid |
| | | | | | | | crystals. |
| 119 | SF₅ | N | N | C—Ph | C—Ph | N | Possible |
| | | | | | | | applications in the |
| | | | | | | | synthesis of |
| | | | | | | | pharmaceuticals, |
| | | | | | | | agrochemicals, |
| | | | | | | | and/or liquid |
| | | | | | | | crystals. |
| 120 | SF₅ | N | C—H | C—COOH | C—H | C—H | Possible |
| | | | | | | | applications in the |
| | | | | | | | synthesis of |
| | | | | | | | pharmaceuticals, |
| | | | | | | | agrochemicals, |
| | | | | | | | and/or liquid |
| | | | | | | | crystals. |
| 121 | SF₄Cl | C—H | O—Bz | C—H | C—H | C—H | Building block for |
| | | | | | | | SF₅— and SF₄R— |
| | | | | | | | containing |
| | | | | | | | compounds. |
| 122 | SF₄Cl | C—H | C—H | O—Bz | C—H | C—H | Building block for |
| | | | | | | | SF₅— and SF₄R— |
| | | | | | | | containing |
| | | | | | | | compounds. |
| 123 | SF₄Cl | C—F | O—Bz | C—H | C—H | C—H | Building block for |
| | | | | | | | SF₅— and SF₄R— |
| | | | | | | | containing |
| | | | | | | | compounds. |
| 124 | SF₄Cl | C—F | C—H | O—Bz | C—H | C—H | Building block for |
| | | | | | | | SF₅— and SF₄R— |
| | | | | | | | containing |
| | | | | | | | compounds. |
| 125 | SF₄Cl | C—F | C—H | C—H | O—Bz | C—H | Building block for |
| | | | | | | | SF₅— and SF₄R— |
| | | | | | | | containing |
| | | | | | | | compounds. |
| 126 | SF₅ | C—H | O—Bz | C—H | C—H | C—H | synthesis of |
| | | | | | | | pharmaceuticals, |
| | | | | | | | agrochemicals, |
| | | | | | | | and/or liquid |
| | | | | | | | crystals. |
| 127 | SF₅ | C—F | O—Bz | C—H | C—H | C—H | synthesis of |
| | | | | | | | pharmaceuticals, |
| | | | | | | | agrochemi cals, |
| | | | | | | | and/or liquid |
| | | | | | | | crystals. |
| 128 | SF₅ | C—F | C—H | C—H | O—Bz | C—H | synthesis of |
| | | | | | | | pharmaceuticals, |
| | | | | | | | agrochemicals, |
| | | | | | | | and/or liquid |
| | | | | | | | crystals. |
| 129 | SF₄Cl | C—H | C—N₃ | C—H | C—H | C—H | Building block for |
| | | | | | | | SF₅— and SF₄R— |
| | | | | | | | containing |
| | | | | | | | compounds. |
| 130 | SF₄Cl | C—H | C—H | C—N₃ | C—H | C—H | Building block for |
| | | | | | | | SF₅— and SF₄R— |
| | | | | | | | containing |
| | | | | | | | compounds. |
| 131 | SF₄Cl | C—F | C—N₃ | C—H | C—H | C—H | Building block for |
| | | | | | | | SF₅— and SF₄R— |
| | | | | | | | containing |
| | | | | | | | compounds. |
| 132 | SF₄Cl | C—F | C—H | C—N₃ | C—H | C—H | Building block for |
| | | | | | | | SF₅— and SF₄R— |
| | | | | | | | containing |
| | | | | | | | compounds. |
| 133 | SF₄Cl | C—F | C—H | C—H | C—N₃ | C—H | Building block for |
| | | | | | | | SF₅— and SF₄R— |
| | | | | | | | containing |
| | | | | | | | compounds. |

All compounds disclosed in the above list may be used for example as synthetic building blocks, pharmaceuticals, agrochemicals and advanced materials such as liquid crystals.

### EXPERIMENTS

### Example 1: General Procedure for Synthesis of Aryl Tetrafluoro-λ⁶-Sulfanyl Chloride Compounds

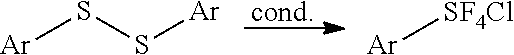

Trichloroisocyanuric acid (0.958 g, 4.1 mmol, 18 equiv.) was added to an oven-dried microwave vial equipped with a stir bar; the vessel was then transported inside a glove box under N₂ atmosphere. Spray-dried (or crushed and rigorously dried) potassium fluoride (0.425 g, 7.3 mmol, 32 equiv.) was added to the reaction vessel, which was then sealed with a cap with septum using a crimper. The closed vial was removed from the glove box. Under Ar atmosphere, a solution of the disulfide substrate (0.23 mmol, 1.0 equiv.) in 1.5 mL MeCN was added to the vial, followed by a solution of trifluoroacetic acid (1.8 microliters, 0.02 mmol, 0.1 equiv.) in 0.5 mL MeCN. The reaction mixture was stirred vigorously at room temperature overnight (ca. 14 h).

Substrates with limited solubility in MeCN were introduced to the reaction mixture as solids in the glove box (and possibly diluted 2-fold to assist stirring). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD₃CN) for ¹⁹F NMR yield determination.

In order to remove KF and TCICA (and its byproducts) outside of the glove box, the crude reaction mixture was first filtered into a polyethylene centrifuge tube and concentrated by blowing N₂ over it. Then, it was diluted with dry pentane, filtered into a polyethylene centrifuge tube, and concentrated by blowing N₂ over it. The crude material consisted of mostly the aryl-SF₄Cl product (amount quantified by ¹⁹F NMR) and was carried forward without further purification.

Alternatively, for more moisture sensitive products, the reaction vessel atmosphere was purged with Ar and transported into the glovebox. Subsequently, the crude reaction mixture was filtered into a PFA vessel via syringe filter and concentrated in vacuo. Then, it was diluted with dry hexanes, filtered into a PFA vessel, and concentrated in vacuo. The crude material consisted of mostly the aryl-SF₄Cl product (amount quantified by ¹⁹F NMR) and was carried forward without further purification.

Representative Product

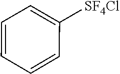

70% yield (by ¹⁹F NMR). The reaction was run according to the general procedure, and the product is consistent with previously reported characterization data. ¹⁹F NMR (377 MHz, CD₃CN): +136.61 (4F, s).

### Example 2: General Procedure for Synthesis of Aryl Sulfur Trifluoride Compounds

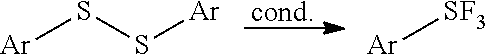

Trichloroisocyanuric acid (0.958 g, 4.1 mmol, 18 equiv.) was added to an oven-dried microwave vial equipped with a stir bar; the vessel was then transported inside a glove box under N₂ atmosphere. Spray-dried (or crushed and rigorously dried) potassium fluoride (0.425 g, 7.3 mmol, 32 equiv.) was added to the reaction vessel, which was then sealed with a cap with septum using a crimper. The closed vial was removed from the glove box. Under Ar atmosphere, a solution of the disulfide substrate (0.23 mmol, 1.0 equiv.) in 1.5 mL MeCN was added to the vial, followed by a solution of trifluoroacetic acid (1.8 microliters, 0.02 mmol, 0.1 equiv.) in 0.5 mL MeCN. The reaction mixture was stirred vigorously at room temperature overnight (ca. 14 h). Note that substrates with limited solubility in MeCN were introduced to the reaction mixture as solids in the glove box (and possibly diluted 2-fold to assist stirring). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD₃CN) for ¹⁹F NMR yield determination.

Representative Product

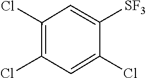

92% yield (by ¹⁹F NMR). The reaction was run according to the general procedure. ¹⁹F NMR (471 MHz, CD₃CN): +63.46 (2F, d, J=75.6 Hz), −56.31 (1F, t, J=75.6 Hz).

### Example 3: General Procedure for Synthesis of Aryl Selenium Trifluoride Compounds

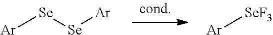

Trichloroisocyanuric acid (0.958 g, 4.1 mmol, 18 equiv.) was added to an oven-dried microwave vial equipped with a stir bar; the vessel was then transported inside a glove box under N₂ atmosphere. Spray-dried (or crushed and rigorously dried) potassium fluoride (0.425 g, 7.3 mmol, 32 equiv.) was added to the reaction vessel, which was then sealed with a cap with septum using a crimper. The closed vial was removed from the glove box. Under Ar atmosphere, a solution of the diselenide substrate (0.23 mmol, 1.0 equiv.) in 1.5 mL MeCN was added to the vial, followed by a solution of trifluoroacetic acid (1.8 microliters, 0.02 mmol, 0.1 equiv.) in 0.5 mL MeCN. The reaction mixture was stirred vigorously at room temperature overnight (ca. 14 h). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD₃CN) for ¹⁹F NMR yield determination.

Representative Product

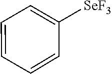

95% yield (by ¹⁹F NMR). The reaction was run according to the general procedure. The product is consistent with previously reported characterization data. ¹⁹F NMR (377 MHz, CD₃CN): −25.51 (3F, br s).

### Example 4: General Procedure for Synthesis of Aryl Pentafluorotelluryl Compounds

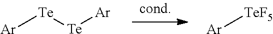

Trichloroisocyanuric acid (0.958 g, 4.1 mmol, 18 equiv.) was added to an oven-dried microwave vial equipped with a stir bar; the vessel was then transported inside a glove box under N₂ atmosphere. Spray-dried (or crushed and rigorously dried) potassium fluoride (0.425 g, 7.3 mmol, 32 equiv.) was added to the reaction vessel, which was then sealed with a cap with septum using a crimper. The closed vial was removed from the glove box. Under Ar atmosphere, a solution of the ditelluride substrate (0.23 mmol, 1.0 equiv.) in 1.5 mL MeCN was added to the vial, followed by a solution of trifluoroacetic acid (1.8 microliters, 0.02 mmol, 0.1 equiv.) in 0.5 mL MeCN. The reaction mixture was stirred vigorously at room temperature overnight (ca. 14 h). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD₃CN) for ¹⁹F NMR yield determination.

Representative Product

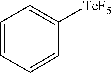

>90% yield (by ¹⁹F NMR). The reaction was run according to the general procedure. The product is consistent with previously reported characterization data. ¹⁹F NMR (282 MHz, CD₃CN): −37.60 (1F, quint, J=148.6 Hz), −54.50 (4F, quint, J=148.6 Hz).

### Example 5: General Procedure for Synthesis of Difluoro(Aryl)(Trifluoromethyl)-λ⁴-Sulfane Compounds

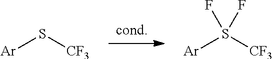

Trichloroisocyanuric acid (0.958 g, 4.1 mmol, 9.0 equiv.) was added to an oven-dried microwave vial equipped with a stir bar; the vessel was then transported inside a glove box under N₂ atmosphere. Spray-dried (or crushed and rigorously dried) potassium fluoride (0.425 g, 7.3 mmol, 16 equiv.) was added to the reaction vessel, which was then sealed with a cap with septum using a crimper. The closed vial was removed from the glove box. Under Ar atmosphere, a solution of the aryl(trifluoromethyl)sulfane substrate (0.46 mmol, 1.0 equiv.) in 1.5 mL MeCN was added to the vial, followed by a solution of trifluoroacetic acid (1.8 microliters, 0.02 mmol, 0.05 equiv.) in 0.5 mL MeCN. The reaction mixture was stirred vigorously at room temperature overnight (ca. 14 h). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD₃CN) for ¹⁹F NMR yield determination.

Representative Product

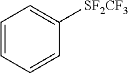

81% yield (by ¹⁹F NMR). The reaction was run according to the general procedure. ¹⁹F NMR (282 MHz, CD₃CN): −14.38 (2F, q, J=18.0 Hz), −62.79 (3F, t, J=18.0 Hz).

### Example 6: General Procedure for Synthesis of Tetrafluoro(Aryl)(Trifluoromethyl)-λ⁶-Tellane Compounds

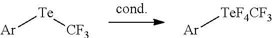

Trichloroisocyanuric acid (0.958 g, 4.1 mmol, 9.0 equiv.) was added to an oven-dried microwave vial equipped with a stir bar; the vessel was then transported inside a glove box under N₂ atmosphere. Spray-dried (or crushed and rigorously dried) potassium fluoride (0.425 g, 7.3 mmol, 16 equiv.) was added to the reaction vessel, which was then sealed with a cap with septum using a crimper. The closed vial was removed from the glove box. Under Ar atmosphere, a solution of the aryl(trifluoromethyl)tellane substrate (0.46 mmol, 1.0 equiv.) in 1.5 mL MeCN was added to the vial, followed by a solution of trifluoroacetic acid (1.8 microliters, 0.02 mmol, 0.05 equiv.) in 0.5 mL MeCN. Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD₃CN) for ¹⁹F NMR yield determination.

Representative Product

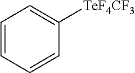

>95% yield (by ¹⁹F NMR). The reaction was run according to the general procedure. ¹⁹F NMR (282 MHz, CD₃CN): −54.10 (3F, quint, J=22.5 Hz), −68.73 (4F, q, J=22.5 Hz).

### Example 8: General Procedure for Synthesis of Aryl Difluoroiodane Compounds

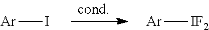

Trichloroisocyanuric acid (0.32 g, 1.4 mmol, 6.0 equiv.) was added to an oven-dried microwave vial equipped with a stir bar; the vessel was then transported inside a glove box under N₂ atmosphere. Spray-dried (or crushed and rigorously dried) potassium fluoride (0.07 g, 1.2 mmol, 5.0 equiv.) was added to the reaction vessel, which was then sealed with a cap with septum using a crimper. The closed vial was removed from the glove box. Under Ar atmosphere, a solution of the aryl iodide substrate (0.23 mmol, 1.0 equiv.) in 2.0 mL MeCN was added to the vial, and the reaction mixture was stirred at 40° C. for ca. 24 h. Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD₃CN) for ¹⁹F NMR yield determination.

Representative Product

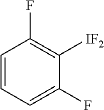

97% yield (by ¹⁹F NMR). The reaction was run according to the general procedure. The product is consistent with previously reported characterization data. ¹⁹F NMR (282 MHz, CD₃CN): −97.44 (2F, t, J=2.3 Hz), −165.67 (2F, t, J=2.3 Hz).

### Example 9: Procedure for Synthesis of SFSCl

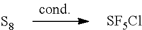

Trichloroisocyanuric acid (0.958 g, 4.1 mmol, 9.0 equiv.) was added to an oven-dried microwave vial equipped with a stir bar; the vessel was then transported inside a glove box under N₂ atmosphere. Spray-dried (or crushed and rigorously dried) potassium fluoride (0.425 g, 7.3 mmol, 16 equiv.) was added to the reaction vessel, followed by elemental sulfur (0.46 mmol, 1.0 equiv.). The reaction vessel was then sealed with a cap with septum using a crimper. The closed vial was removed from the glove box. Under Ar atmosphere, 1.5 mL MeCN was added to the vial, followed by a solution of trifluoroacetic acid (1.8 microliters, 0.02 mmol, 0.05 equiv.) in 0.5 mL MeCN. The reaction mixture was stirred vigorously at room temperature overnight (ca. 14 h). Upon reaction completion, the head space of the vial was drawn up into a syringe for GC/MS analysis. Subsequently, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD₃CN) for ¹⁹F NMR analysis.

The product is consistent with previously reported characterization data. ¹⁹F NMR (282 MHz, CD₃CN): 124.26 (4F, d, J=151.2 Hz), 64.15 (1F, d, J=151.2 Hz).

### Example 10: General Procedure for Synthesis of

Pentafluorosulfanyl Compounds

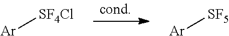

A solution of a known amount of aryl-SF₄Cl compound (1.0 equiv.) in anhydrous CH₂Cl₂ was transferred to a copper (or PFA) vessel and concentrated. Subsequently, AgF (2.0 equiv.) was added, and the reactor was sealed under Ar atmosphere. The sealed reactor was heated to 120° C. for ca. 2 days. Upon cooling, the reactor was rinsed with copious amounts of CH₂Cl₂ and H₂O into a separatory funnel. The reaction mixture was extracted with CH₂Cl₂. The combined organic layers were dried with MgSO₄, filtered through Celite, and concentrated. The crude reaction mixture was purified via gradient column chromatography on a Teledyne-Isco Combiflash instrument, eluting with hexanes:EtOAc.

Representative Product

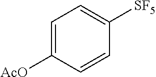

77% yield (isolated). The reaction was run according to the general procedure using AgF in a copper vessel; the product was isolated via gradient column chromatography on silica gel in as a white solid. ¹⁹F NMR (377 MHz, CDCl₃): 84.32 (1F, quint, J=150.6 Hz), 63.62 (4F, d, J=150.6 Hz); ¹H NMR (400 MHz, CDCl₃): 7.78 (2H, dm, J=9.1 Hz), 7.20 (2H, d, J=9.1 Hz), 2.33 (3H, s); ¹³C {¹H} NMR (101 MHz, CDCl₃): 168.7, 152.5, 150.9 (quint, J=18.0 Hz), 127.5 (quint, J=4.8 Hz), 121.8, 21.0.

### Example 11: General Procedure for Synthesis of Aryl Tetrafluoro-λ⁶-sulfanyl Chloride Alkanes/Alkenes

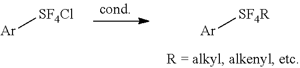

A solution of a known amount of aryl-SF₄Cl compound (1.0 equiv.) in anhydrous CH₂Cl₂ (0.05-0.1 M) was transferred to a PFA vessel equipped with a stir bar under Ar atmosphere. The alkene or alkyne substrate (1.5 equiv.) was added, followed by 10 mol % BEt₃ (administered as a 1.0 M solution in hexanes), and the reaction mixture was stirred at room temperature for 1 h. At this time, the reaction mixture was quenched with saturated aq. NaHCO₃ and extracted into CH₂Cl₂. The combined organic layers were dried with MgSO₄, filtered through Celite, and concentrated. The crude reaction mixture was purified via gradient column chromatography on a Teledyne-Isco Combiflash instrument, eluting with hexanes:EtOAc.

Representative Products

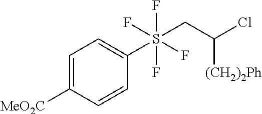

84% yield (isolated). The reaction was run according to the general procedure using 4-phenyl-1-butene and BEt₃; the product was isolated via gradient column chromatography on silica gel as a white solid. ¹⁹F NMR (377 MHz, CDCl₃): 57.59 (4F, t, J=8.5 Hz, becomes s in ¹⁹F{¹H} spectrum); ¹H NMR (400 MHz, CDCl₃): 9.10 (1H, d, J=2.1 Hz), 8.44 (1H, d, J=8.5 Hz), 7.80 (1H, d, J=8.5 Hz), 7.34-7.21 (5H, m), 4.60-4.54 (1H, m), 4.46-4.34 (1H, m, becomes dd, J=13.7, 5.3 Hz in ¹H{¹⁹F} spectrum), 4.33-4.20 (1H, m, becomes dd, J=13.7, 7.2 Hz in ¹H{¹⁹F} spectrum), 4.00 (3H, s), 3.00 (1H, ddd, J=14.0, 9.2, 4.5 Hz), 2.87-2.80 (1H, m), 2.52-2.44 (1H, m), 2.18-2.08 (1H, m); ¹³C{¹H} NMR (101 MHz, CDCl₃): 172.6 (quint, J=31.7 Hz), 164.3, 148.6 (m), 140.2, 139.6, 128.53, 128.49, 127.9, 126.3, 121.1 (quint, J=4.8 Hz), 81.6 (quint, J=18.7 Hz), 56.5 (quint, J=5.2 Hz), 52.8, 39.2, 32.3.

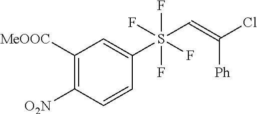 [SMILES: COC(=O)c1cc(S(F)(F)(F)(F)/C=C(/Cl)c2ccccc2)ccc1[N+](=O)[O-]]

70% yield (isolated). The reaction was run according to the general procedure using phenylacetylene and BEt₃; the product was isolated via gradient column chromatography on silica gel as a white solid. ¹⁹F NMR (282 MHz, CD₃CN): 71.26 (4F, d, J=8.4 Hz, becomes s in ¹⁹F{¹H} spectrum); ¹H NMR (400 MHz, CDCl₃): 8.01 (1H, dm, J=2.2 Hz), 7.86 (1H, dd, J=8.9, 2.2 Hz), 7.81 (1H, dm, J=8.9 Hz), 7.43-7.38 (5H, m), 7.18 (1H, quint, J=8.4 Hz), 3.91 (3H, s); ¹³C{¹H} NMR (101 MHz, CDCl₃): 164.2, 161.7 (quint, J=27.6 Hz), 148.6, 143.0 (quint, J=28.6 Hz), 139.8 (quint, J=7.8 Hz), 136.5, 129.7 (quint, J=5.4 Hz), 129.5, 128.1, 127.9 (m), 127.2, 123.8, 53.6.

### Example 12: Representative Procedure for Synthesis of Difluoro(aryl)(trifluoromethyl)-λ⁴-sulfane Compound and Application as Putative Nucleophilic Fluorinating Reagent

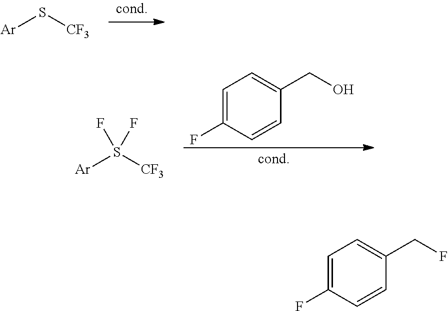

Trichloroisocyanuric acid (0.958 g, 4.1 mmol, 9.0 equiv.) was added to an oven-dried microwave vial equipped with a stir bar; the vessel was then transported inside a glove box under N₂ atmosphere. Spray-dried (or crushed and rigorously dried) potassium fluoride (0.425 g, 7.3 mmol, 16 equiv.) was added to the reaction vessel, which was then sealed with a cap with septum using a crimper. The closed vial was removed from the glove box. Under Ar atmosphere, a solution of the aryl(trifluoromethyl)sulfane substrate (0.46 mmol, 1.0 equiv.) in 1.5 mL MeCN was added to the vial, followed by a solution of trifluoroacetic acid (1.8 microliters, 0.02 mmol, 0.05 equiv.) in 0.5 mL MeCN. The reaction mixture was stirred vigorously at room temperature overnight (ca. 14 h). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD₃CN) for ¹⁹F NMR yield determination.

In order to remove KF and TCICA (and its byproducts) outside of the glove box, the crude reaction mixture was first filtered into a polyethylene centrifuge tube and concentrated by blowing N₂ over it. Then, it was diluted with dry pentane, filtered into a polyethylene centrifuge tube, and concentrated by blowing N₂ over it. The crude material consisted of mostly the aryl-SF₄Cl product (amount quantified by ¹⁹F NMR) and was carried forward without further purification (˜0.34 mmol isolated aryl-SF₂CF₃ based on ¹⁹F NMR analysis).

A solution of the difluoro(aryl)(trifluoromethyl)-λ⁴-sulfane substrate (˜0.34 mmol, 1.0 equiv.) in 4 mL CHCl₃ was added to an oven-dried microwave vial equipped with a stir bar and sealed with a cap with septum under Ar atmosphere. Subsequently, 4-fluorobenzyl alcohol (0.04 mL, 0.37 mmol, 1.1 equiv.) was added to the vial, and the reaction mixture was stirred at room temperature. After 45 min, an aliquot was taken from the reaction mixture for ¹⁹F NMR analysis. (Note: trifluorotoluene was added to the solution as an internal reference, but not for quantification purposes.) Representative Products

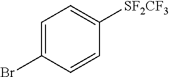

77% yield (by ¹⁹F NMR). The reaction was run according to the representative procedure. ¹⁹F NMR (282 MHz, CD₃CN): −13.99 (2F, q, J=17.9 Hz), −62.77 (3F, t, J=17.9 Hz).

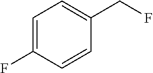

The reaction was run according to the representative procedure. ¹⁹F NMR (282 MHz, CD₃CN): −113.51 (1F, m), −203.83 (1F, t, J=48.1 Hz).

### Example 13: General Procedure for Synthesis of Trifluoromethyl Tetrafluoro-λ6-Sulfanyl Chloride

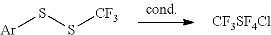

Trichloroisocyanuric acid (0.958 g, 4.1 mmol, 18 equiv.) was added to an oven-dried microwave vial equipped with a stir bar; the vessel was then transported inside a glove box under N2 atmosphere. Spray-dried (or crushed and rigorously dried) potassium fluoride (0.425 g, 7.3 mmol, 32 equiv.) was added to the reaction vessel, which was then sealed with a cap with septum using a crimper. The closed vial was removed from the glove box. Under Ar atmosphere, a solution of the disulfide substrate (0.23 mmol, 1.0 equiv.) in 1.5 mL MeCN was added to the vial, followed by a solution of trifluoroacetic acid (1.8 microliters, 0.02 mmol, 0.1 equiv.) in 0.5 mL MeCN. The reaction mixture was stirred vigorously at room temperature overnight (ca. 14 h). Substrates with limited solubility in MeCN were introduced to the reaction mixture as solids in the glove box (and possibly diluted 2-fold to assist stirring). Upon reaction completion, an aliquot of the reaction mixture was passed through a PTFE syringe filter, and an NMR sample was prepared for 19F NMR analysis.

The product (synthesized from 1-(4-nitrophenyl)-2-(trifluoromethyl)disulfide) is consistent with previously reported characterization data. 19F NMR (282 MHz, CD3CN): trans-isomer: +102.88 (4F, q, J=22.2 Hz), −65.39 (3F, quint, J=22.2 Hz); cis-isomer: +134.48 (1F, qq, J=146.6, 9.1 Hz), +83.55 (2F, ddq, J=146.6, 102.9, 19.7 Hz), +40.83 (1F, dtq, J=146.6, 102.9, 22.8 Hz), −65.95 (3F, dtd, J=22.8, 19.7, 9.1 Hz). cis:trans ratio: 3:1.

### Example 14: General Procedure for Synthesis of Diaryl Tetrafluoro-λ6-tellane Compounds

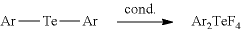

Trichloroisocyanuric acid (0.319 g, 1.4 mmol, 3.0 equiv.) was added to an oven-dried microwave vial equipped with a stir bar; the vessel was then transported inside a glove box under N2 atmosphere. Spray-dried (or crushed and rigorously dried) potassium fluoride (0.319 g, 5.5 mmol, 12 equiv.) was added to the reaction vessel, which was then sealed with a cap with septum using a crimper. The closed vial was removed from the glove box. Under Ar atmosphere, a solution of the diaryl monotelluride substrate (0.46 mmol, 1.0 equiv.) in 1.5 mL MeCN was added to the vial, followed by a solution of trifluoroacetic acid (1.8 microliters, 0.02 mmol, 0.1 equiv.) in 0.5 mL MeCN. The reaction mixture was stirred vigorously at room temperature overnight (ca. 20 h). Substrates with limited solubility in MeCN were introduced to the reaction mixture as solids in the glove box (and possibly diluted 2-fold to assist stirring). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD3CN) for 19F NMR yield determination.

Representative Product

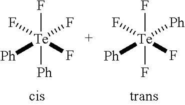

39% trans and 6% cis observed by 19F NMR. The products are consistent with previously reported characterization data. 19F NMR (282 MHz, CD3CN): trans-isomer: −58.11 (4F, s); cis-isomer: −37.07 (2F, t, J=87.5 Hz), −77.29 (2F, t, J=87.5 Hz).

### Example 15: General Procedure for Synthesis of Aryl Tetrafluoro-λ6-Sulfanyl Chloride Compounds

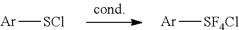

Trichloroisocyanuric acid (0.958 g, 4.1 mmol, 9.0 equiv.) was added to an oven-dried microwave vial equipped with a stir bar; the vessel was then transported inside a glove box under N2 atmosphere. Spray-dried (or crushed and rigorously dried) potassium fluoride (0.425 g, 7.3 mmol, 18 equiv.) was added to the reaction vessel, which was then sealed with a cap with septum using a crimper. The closed vial was removed from the glove box. Under Ar atmosphere, a solution of the sulfenyl chloride substrate (0.46 mmol, 1.0 equiv.) in 1.5 mL MeCN was added to the vial, followed by a solution of trifluoroacetic acid (1.8 microliters, 0.02 mmol, 0.05 equiv.) in 0.5 mL MeCN. The reaction mixture was stirred vigorously at room temperature overnight (ca. 14 h). Substrates with limited solubility in MeCN were introduced to the reaction mixture as solids in the glove box (and possibly diluted 2-fold to assist stirring). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD3CN) for 19F NMR yield determination.

Representative Product

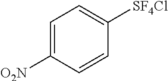

68% yield by 19F NMR. The product (synthesized from 4-nitrobenzenesulfenyl chloride) is consistent with previously reported characterization data. 19F NMR (282 MHz, CD3CN): +135.02 (4F, s).

### Example 16: General Procedure for Synthesis of Aryl Tetrafluoro-λ6-Sulfanyl Chloride Compounds

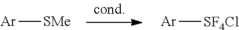

Trichloroisocyanuric acid (0.958 g, 4.1 mmol, 9.0 equiv.) was added to an oven-dried microwave vial equipped with a stir bar; the vessel was then transported inside a glove box under N2 atmosphere. Spray-dried (or crushed and rigorously dried) potassium fluoride (0.425 g, 7.3 mmol, 18 equiv.) was added to the reaction vessel, which was then sealed with a cap with septum using a crimper. The closed vial was removed from the glove box. Under Ar atmosphere, a solution of the aryl methyl sulfide substrate (0.46 mmol, 1.0 equiv.) in 1.5 mL MeCN was added to the vial, followed by a solution of trifluoroacetic acid (1.8 microliters, 0.02 mmol, 0.05 equiv.) in 0.5 mL MeCN. The reaction mixture was stirred vigorously at room temperature overnight (ca. 14 h). Substrates with limited solubility in MeCN were introduced to the reaction mixture as solids in the glove box (and possibly diluted 2-fold to assist stirring). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD3CN) for 19F NMR yield determination.

Representative Product

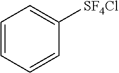

The product is consistent with previously reported characterization data. 19F NMR (282 MHz, CD3CN): +136.61 (4F, s).

### Example 17: General Procedure for Synthesis of Difluoro(Aryl)(Trifluoromethyl)-λ4-Selane Compounds

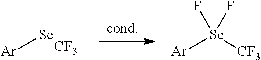

Trichloroisocyanuric acid (0.958 g, 4.1 mmol, 9.0 equiv.) was added to an oven-dried microwave vial equipped with a stir bar; the vessel was then transported inside a glove box under N2 atmosphere. Spray-dried (or crushed and rigorously dried) potassium fluoride (0.425 g, 7.3 mmol, 16 equiv.) was added to the reaction vessel, which was then sealed with a cap with septum using a crimper. The closed vial was removed from the glove box. Under Ar atmosphere, a solution of the aryl(trifluoromethyl)selane substrate (0.46 mmol, 1.0 equiv.) in 1.5 mL MeCN was added to the vial, followed by a solution of trifluoroacetic acid (1.8 microliters, 0.02 mmol, 0.05 equiv.) in 0.5 mL MeCN. The reaction mixture was stirred vigorously at room temperature overnight (ca. 14 h). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD3CN) for 19F NMR yield determination.

Representative Product

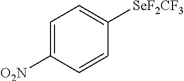

71% yield (by 19F NMR). The reaction was run according to the general procedure. 19F NMR (282 MHz, CD3CN): −58.30 (3F, t, J=12.2 Hz), −73.21 (4F, t, J=12.2 Hz).

### Example 18: General Procedure for Synthesis of Tetrafluoro(Aryl)-λ5-Iodane Compounds

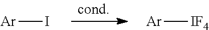

Trichloroisocyanuric acid (0.350 g, 1.5 mmol, 4.0 equiv.) was added to an oven-dried microwave vial equipped with a stir bar; the vessel was then transported inside a glove box under N2 atmosphere. Spray-dried (or crushed and rigorously dried) potassium fluoride (0.131 g, 2.3 mmol, 6.0 equiv.) was added to the reaction vessel, which was then sealed with a cap with septum using a crimper. The closed vial was removed from the glove box. Under Ar atmosphere, a solution of the aryl iodide substrate (0.38 mmol, 1.0 equiv.) in 4.0 mL MeCN was added to the vial. The reaction mixture was stirred vigorously at room temperature for ca. 48 h. Substrates with limited solubility in MeCN were introduced to the reaction mixture as solids in the glove box (and possibly diluted 2-fold to assist stirring). Upon reaction completion, an aliquot of the reaction mixture was passed through a PTFE syringe filter, and an NMR sample was prepared with 0.4 mL of the filtered aliquot+0.1 mL internal standard solution (made immediately prior to use with x g of trifluorotoluene in y mL CD3CN) for 19F NMR yield determination.

Representative Product

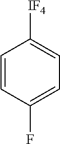

85% yield by 19F NMR. The product is consistent with previously reported characterization data. 19F NMR (282 MHz, CD3CN): −25.86 (4F, br s), −104.29 to −104.46 (1F, m).

The following compounds were synthesized using the reaction conditions described above:

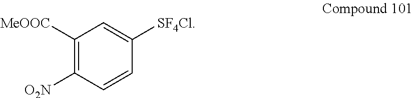

The reaction was run according to the general procedure, and the product was converted to the more stable aryl tetrafluoro-λ⁶-sulfanyl alkene 232 to obtain complete characterization data. ¹⁹F NMR (282 MHz, CD₃CN): +134.63 (4F, s).

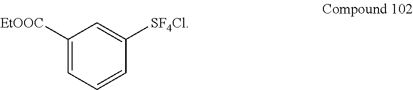

The reaction was run according to the general procedure, and the product was converted to the more stable pentafluorosulfanyl arene 227 to obtain complete characterization data. ¹⁹F NMR (282 MHz, CD₃CN): +135.95 (4F, s)

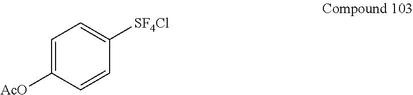

The reaction was run according to the general procedure, and the product was converted to the more stable pentafluorosulfanyl arene 111 to obtain complete characterization data. ¹⁹F NMR (282 MHz, CD₃CN): +137.43 (4F, s).

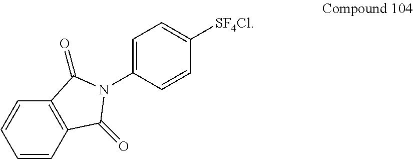

The reaction was run according to the general procedure. ¹⁹F NMR (282 MHz, CD₃CN): +136.81 (4F, s) Compound 105.

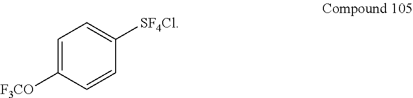

The reaction was run according to the general procedure. ¹⁹F NMR (282 MHz, CD₃CN): +136.73 (4F, s), −58.56 (3F, s)

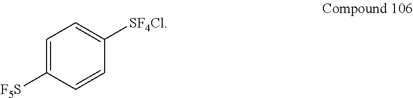

The reaction was run according to the general procedure. ¹⁹F NMR (377 MHz, CD₃CN): +134.96 (4F, s), +81.54 (1F, quint, J=148.5 Hz), +61.86 (4F, d, J=148.5 Hz).

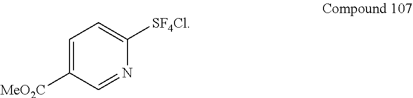

The reaction was run according to the general procedure, and the product was converted to the more stable aryl tetrafluoro-λ⁶-sulfanyl alkane 228 to obtain complete characterization data. ¹⁹F NMR (282 MHz, CD₃CN): +123.52 (4F, s).

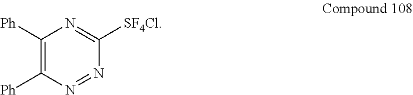

The reaction was run according to the general procedure. ¹⁹F NMR (377 MHz, CD₃CN): +120.59 (4F, s)

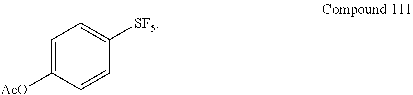

The reaction was run according to the general procedure using AgF in a copper vessel; the product was isolated via gradient column chromatography on silica gel in 77% yield (46 mg, 0.18 mmol) as a white solid. ¹⁹F NMR (377 MHz, CDCl₃): 84.32 (1F, quint, J=150.6 Hz), 63.62 (4F, d, J=150.6 Hz); ¹H NMR (400 MHz, CDCl₃): 7.78 (2H, dm, J=9.1 Hz), 7.20 (2H, d, J=9.1 Hz), 2.33 (3H, s); ¹³C{¹H} NMR (101 MHz, CDCl₃): 168.7, 152.5, 150.9 (quint, J=18.0 Hz), 127.5 (quint, J=4.8 Hz), 121.8, 21.0.

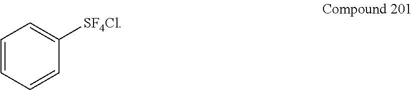

The reaction was run according to the general procedure, and the product is consistent with previously reported characterization data. ¹⁹F NMR (377 MHz, CD₃CN): +136.61 (4F, s).

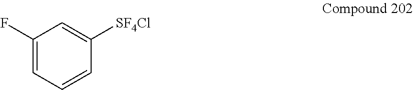

The reaction was run according to the general procedure, and the product is consistent with previously reported characterization data. ¹⁹F NMR (282 MHz, CD₃CN): +136.08 (4F, s), −111.34 (1F, m).

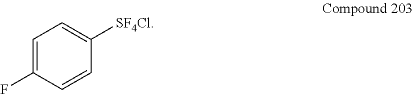

The reaction was run according to the general procedure, and the product is consistent with previously reported characterization data. ¹⁹F NMR (377 MHz, CD₃CN): +137.65 (4F, s), −108.21 (1F, m).

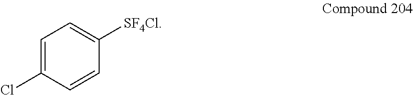

The reaction was run according to the general procedure, and the product is consistent with previously reported characterization data. ¹⁹F NMR (282 MHz, CD₃CN): +136.75 (4F, s).

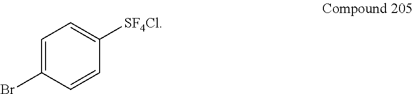

The reaction was run according to the general procedure, and the product is consistent with previously reported characterization data. ¹⁹F NMR (282 MHz, CD₃CN): +136.59 (4F, s).

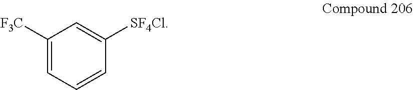

The reaction was run according to the general procedure, and the product is consistent with previously reported characterization data. ¹⁹F NMR (377 MHz, CD₃CN): +135.61 (4F, s), −63.21 (3F, s)

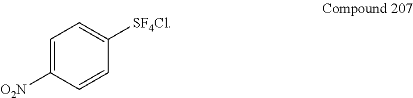

The reaction was run according to the general procedure, and the product is consistent with previously reported characterization data. ¹⁹F NMR (377 MHz, CD₃CN): +135.02 (4F, s)

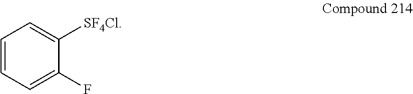

The reaction was run according to the general procedure, and the product is consistent with previously reported characterization data. ¹⁹F NMR (282 MHz, CD₃CN): +140.30 (4F, d, J=24.5 Hz), −110.04 (1F, m)

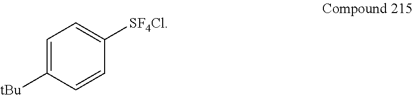

The reaction was run according to the general procedure, and the product is consistent with previously reported characterization data. ¹⁹F NMR (282 MHz, CD₃CN): +137.64 (4F, s)

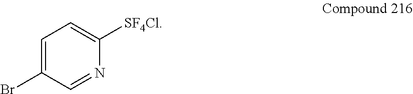

The reaction was run according to the general procedure, and the product is consistent with previously reported characterization data. ¹⁹F NMR (282 MHz, CD₃CN): +124.66 (4F, s).

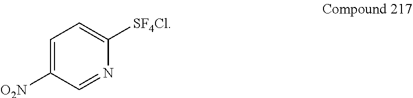

The reaction was run according to the general procedure, and the product is consistent with previously reported characterization data. ¹⁹F NMR (377 MHz, CD₃CN): +123.42 (4F, s)

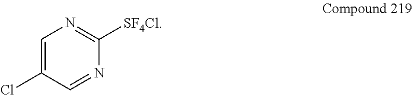

The reaction was run according to the general procedure, and the product is consistent with previously reported characterization data. ¹⁹F NMR (282 MHz, CD₃CN): +119.06 (4F, s).

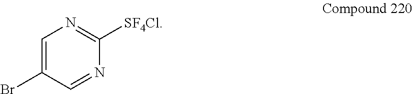

The reaction was run according to the general procedure, and the product is consistent with previously reported characterization data. ¹⁹F NMR (282 MHz, CD₃CN): +118.97 (4F, s).

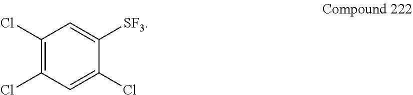

The reaction was run according to the general procedure; the product was unstable toward isolation and characterized by ¹⁹F NMR. ¹⁹F NMR (471 MHz, CD₃CN): +63.46 (2F, d, J=75.6 Hz), −56.31 (1F, t, J=75.6 Hz).

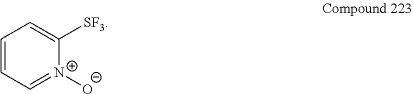

The reaction was run according to the general procedure; the product was unstable toward isolation and characterized by ¹⁹F NMR. ¹⁹F NMR (377 MHz, CD₃CN)+53.58 (2F, d, J=102.2 Hz), −67.65 (1F, t, J=102.2 Hz).

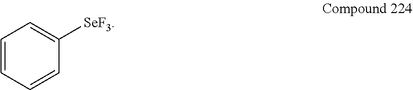

The reaction was run according to the general procedure; the product was unstable toward isolation and characterized by ¹⁹F NMR. The product is consistent with previously reported characterization data. ¹⁹F NMR (377 MHz, CD₃CN): −25.51 (3F, br s).

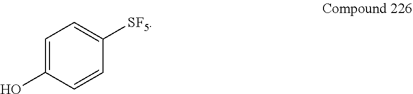

The reaction was run according to the general procedure using AgF in a copper vessel followed by the LiOH workup modification; the product was isolated via gradient column chromatography on silica gel in 68% yield (21 mg, 0.10 mmol) as a white solid. The product is consistent with previously reported characterization data. ¹⁹F NMR (377 MHz, CDCl₃): 86.05 (1F, quint, J=150.0 Hz), 64.32 (4F, d, J=150.0 Hz); ¹H NMR (400 MHz, CDCl₃): 7.65 (2H, dm, J=9.1 Hz), 6.86 (2H, dm, J=9.1 Hz), 5.17 (1H, br s).

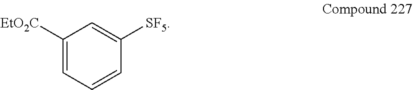

The reaction was run according to the general procedure using AgF in a copper vessel; the product was isolated via gradient column chromatography on silica gel in 57% yield (20 mg, 0.07 mmol) as a colorless oil. ¹⁹F NMR (471 MHz, CDCl₃): 83.35 (1F, quint, J=150.4 Hz), 62.79 (4F, d, J=150.4 Hz); ¹H NMR (500 MHz, CDCl₃): 8.43 (1H, m), 8.20 (1H, d, J=7.8 Hz), 7.94 (1H, m), 7.56 (1H, t, J=8.0 Hz), 4.43 (2H, q, J=7.1 Hz), 1.42 (3H, t, J=7.1 Hz); ¹³C{¹H} NMR (126 MHz, CDCl₃): 164.8, 153.9 (quint, J=18.2 Hz), 132.5, 131.5, 130.0 (quint, J=4.6 Hz), 128.9, 127.2 (quint, J=4.6 Hz), 61.8, 14.3.

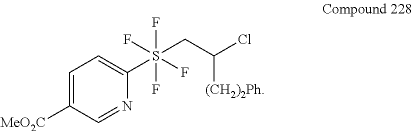

The reaction was run according to the general procedure using 4-phenyl-1-butene and BEt₃; the product was isolated via gradient column chromatography on silica gel in 84% yield (25 mg, 0.06 mmol) as a white solid. Although this product proved stable toward column chromatography, note that it degraded after a few days in CDCl₃ solution in the NMR tube. ¹⁹F NMR (377 MHz, CDCl₃): 57.59 (4F, t, J=8.5 Hz, becomes s in ¹⁹F{¹H} spectrum); ¹H NMR (400 MHz, CDCl₃): 9.10 (1H, d, J=2.1 Hz), 8.44 (1H, d, J=8.5 Hz), 7.80 (1H, d, J=8.5 Hz), 7.34-7.21 (5H, m), 4.60-4.54 (1H, m), 4.46-4.34 (1H, m, becomes dd, J=13.7, 5.3 Hz in ¹H{¹⁹F} spectrum), 4.33-4.20 (1H, m, becomes dd, J=13.7, 7.2 Hz in ¹H{¹⁹F} spectrum), 4.00 (3H, s), 3.00 (1H, ddd, J=14.0, 9.2, 4.5 Hz), 2.87-2.80 (1H, m), 2.52-2.44 (1H, m), 2.18-2.08 (1H, m); ¹³C{¹H} NMR (101 MHz, CDCl₃): 172.6 (quint, J=31.7 Hz), 164.3, 148.6 (m), 140.2, 139.6, 128.53, 128.49, 127.9, 126.3, 121.1 (quint, J=4.8 Hz), 81.6 (quint, J=18.7 Hz), 56.5 (quint, J=5.2 Hz), 52.8, 39.2, 32.3.

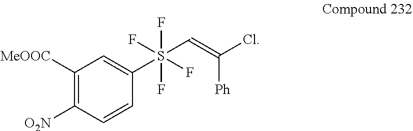 [SMILES: COC(=O)c1cc(S(F)(F)(F)(F)/C=C(/Cl)c2ccccc2)ccc1[N+](=O)[O-]]

The reaction was run according to the general procedure using phenylacetylene and BEt₃; the product was isolated via gradient column chromatography on silica gel in 70% yield (40 mg, 0.09 mmol) as a white solid. Although this product proved stable toward column chromatography, note that it degraded after a few days in CDCl₃ solution in the NMR tube. ¹⁹F NMR (282 MHz, CD₃CN): 71.26 (4F, d, J=8.4 Hz, becomes s in ¹⁹F{¹H} spectrum); ¹H NMR (400 MHz, CDCl₃): 8.01 (1H, dm, J=2.2 Hz), 7.86 (1H, dd, J=8.9, 2.2 Hz), 7.81 (1H, dm, J=8.9 Hz), 7.43-7.38 (5H, m), 7.18 (1H, quint, J=8.4 Hz), 3.91 (3H, s); ¹³C{¹H} NMR (101 MHz, CDCl₃): 164.2, 161.7 (quint, J=27.6 Hz), 148.6, 143.0 (quint, J=28.6 Hz), 139.8 (quint, J=7.8 Hz), 136.5, 129.7 (quint, J=5.4 Hz), 129.5, 128.1, 127.9 (m), 127.2, 123.8, 53.6.

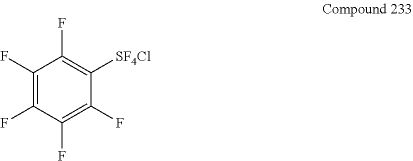

The reaction was run according to the general procedure, and the product is consistent with previously reported characterization data. ¹⁹F NMR (282 MHz, CD₃CN): trans-isomer: +143.21 (4F, t, J=27.6 Hz), −135.35 (2F, m), −148.85 (1F, m), −161.05 (2F, m); cis-isomer: +153.07 (1F, q, J=158.3 Hz), +122.77 (2F, ddd, J=158.3, 95.1, 78.2 Hz), +79.21 (1F, dtt, J=158.3, 95.1, 20.9 Hz), −135.35 (2F, m), −148.85 (1F, m), −161.05 (2F, m) trans:cis ratio: 1.5:1.

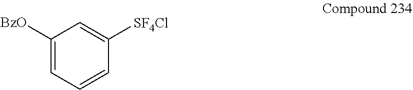

The reaction was run according to the general procedure, and the product was converted to the more stable pentafluorosulfanyl arene to obtain complete characterization data. ¹⁹F NMR (282 MHz, CD₃CN): +136.39 (4F, s)

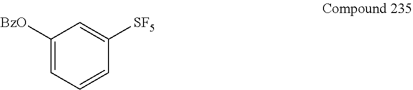

The reaction was run according to the general procedure using 4.0 equiv. AgF in a PFA vessel; the product was isolated via gradient column chromatography on silica gel in 81% yield (23 mg, 0.07 mmol) as a yellow oil. ¹⁹F NMR (471 MHz, CDCl₃): 83.53 (1F, quint, J=150.8 Hz), 63.08 (4F, d, J=150.8 Hz); ¹H NMR (500 MHz, CDCl₃): 8.22-8.20 (2H, m), 7.70-7.66 (3H, m), 7.56-7.53 (3H, m), 7.44-7.43 (1H, m); ¹³C{¹H} NMR (126 MHz, CDCl₃): 164.6, 154.3 (quint, J=18.2 Hz), 150.5, 134.1, 130.3, 129.5, 128.7, 125.3, 123.4 (quint, J=4.6 Hz), 120.1 (quint, J=4.6 Hz). □ₘₐₓ (ATR-IR): 1743 cm⁻¹. HRMS (ESI-TOF): calc'd for C₁₃H₉F₅NaO₂S [M+Na]⁺: 347.0136, found: 347.0131.

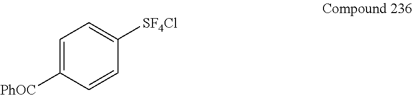

The reaction was run according to the general procedure, and the product was converted to the more stable pentafluorosulfanyl arene to obtain complete characterization data. ¹⁹F NMR (282 MHz, CD₃CN): +135.78.

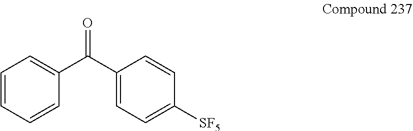

The reaction was run according to the general procedure using AgF in a PFA vessel; the product was isolated via gradient column chromatography on silica gel in 57% yield (18 mg, 0.06 mmol) as a white solid; m.p. 116.4-117.3° C. ¹⁹F NMR (377 MHz, CDCl₃): 83.11 (1F, quint, J=150.4 Hz), 62.64 (4F, d, J=150.4 Hz); ¹H NMR (400 MHz, CDCl₃): 7.90-7.85 (4H, m), 7.82-7.79 (2H, m), 7.66-7.62 (1H, tm, J=7.4 Hz), 7.54-7.49 (2H, m); ¹³C{¹H}NMR (101 MHz, CDCl₃): 194.9, 156.2 (quint, J=18.1 Hz), 140.3, 136.5, 133.3, 130.09, 130.08, 128.6, 126.1 (quint, J=4.7 Hz). □ₘₐₓ (ATR-IR): 1653 cm⁻¹. HRMS (EI) calculated for Cl₃H₉F₅OS [M]⁺: 308.0289, found: 308.0282.

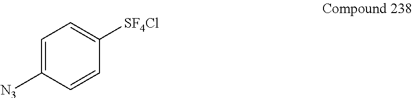

The reaction was run according to the general procedure, and the product was converted to the more stable pentafluorosulfanyl arene to obtain complete characterization data. ¹⁹F NMR (282 MHz, CD₃CN): +137.77 (4F, s).

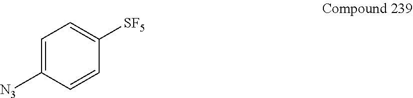

The reaction was run according to the general procedure using AgF in a PFA vessel; the product was isolated via gradient column chromatography on silica gel in 63% yield (21.3 mg, 0.09 mmol) as a light yellow oil. ¹⁹F NMR (471 MHz, CDCl₃): 84.59 (1F, quint, J=150.8 Hz), 63.67 (4F, quint, J=150.8 Hz); ¹H NMR (500 MHz, CDCl₃): 7.74 (2H, d, J=9.0 Hz), 7.08 (2H, d, J=9.0 Hz). The product is consistent with previously reported characterization data.

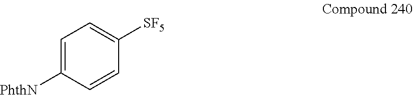

The reaction was run according to the general procedure using 4.0 equiv. AgF in a PFA vessel; the product was isolated via gradient column chromatography on silica gel in 80% yield (6.9 mg, 0.02 mmol) as a white solid; m.p. 217.2-219.0° C. ¹⁹F NMR (471 MHz, CDCl₃): 83.79 (1F, quint, J=150.5 Hz), 63.14 (4F, d, J=150.5 Hz); ¹H NMR (500 MHz, CDCl₃): 7.99 (2H, dd, J=5.4, 3.1 Hz), 7.90 (2H, d, J=9.1 Hz), 7.84 (2H, dd, J=5.4, 3.1 Hz), 7.65 (2H, d, J=9.1 Hz); ¹³C{¹H} NMR (126 MHz, CDCl₃): 166.6, 152.5 (quint, J=18.2 Hz), 134.8, 134.6, 131.4, 126.9 (quint, J=4.5 Hz), 126.1, 124.1. □ₘₐₓ (ATR-IR): 1720, 1711, 1702 cm⁻¹. HRMS (ESI-TOF): calc'd for C₁₄H₉F₅NO₂S [M+H]⁺: 350.0269, found: 350.0268. The product is consistent with previously reported characterization data.

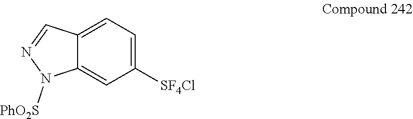

The reaction was run according to the general procedure. ¹⁹F NMR (282 MHz, CD₃CN): +137.59 (4F, s).

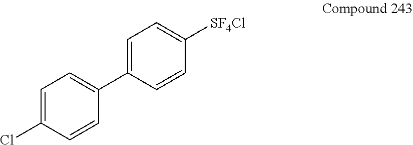

The reaction was run according to the general procedure. ¹⁹F NMR (282 MHz, CD₃CN): +137.13 (4F, s)

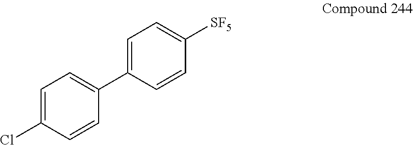

The reaction was run according to the general procedure using AgF in a PFA vessel; the product was isolated via gradient column chromatography on silica gel in 59% yield (20 mg, 0.06 mmol) as a white solid; m.p. 82.8-84.8° C. ¹⁹F NMR (471 MHz, CDCl₃): +84.60 (1F, quint, J=150.2 Hz), +63.24 (4F, d, J=150.2 Hz); ¹H NMR (500 MHz, CDCl₃): 7.83 (2H, dm, J=8.6 Hz), 7.62 (2H, br d, J=8.6 Hz), 7.52 (2H, dm, J=8.6 Hz), 7.45 (2H, dm, J=8.6 Hz); ¹³C{¹H} NMR (126 MHz, CDCl₃): 153.1 (quint, J=17.5 Hz), 143.3, 137.5, 134.8, 129.3, 128.5, 127.1, 126.6 (quint, J=4.6 Hz). □ₘₐₓ (ATR-IR): 840 cm⁻¹ (br), 813 cm⁻¹.

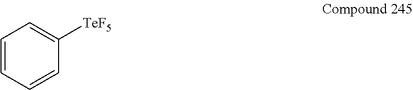

The reaction was run according to the general procedure, and the product is consistent with previously reported characterization data. Colorless oil. ¹⁹F NMR (282 MHz, CDCl₃): −37.11 (1F, quint, J=150.6 Hz), −53.39 (4F, d, J=150.6 Hz); ¹H NMR (400 MHz, CDCl₃): 7.92 (2H, d, J=8.1 Hz), 7.83-7.78 (1H, m), 7.75-7.70 (2H, m); ¹³C{¹H} NMR (101 MHz, CDCl₃): 142.2-141.9 (m), 135.4, 131.4 (quint, J=1.5 Hz), 130.3 (quint, J=2.2 Hz). □ₘₐₓ (ATR-IR): 655 cm⁻¹ (br). HRMS (EI): calc'd for C₆H₅F₅Te [M]⁺: 301.9374, found: 301.9374.

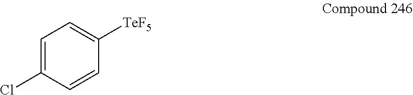

The reaction was run according to the general procedure. Clear solid; m.p. 75.4-76.3° C. ¹⁹F NMR (282 MHz, CDCl₃): −37.25 (1F, quint, J=151.7 Hz), −52.22 (4F, d, J=151.7 Hz); ¹H NMR (400 MHz, CDCl₃): 7.88 (2H, d, J=8.7 Hz), 7.71 (2H, dquint, J=8.7, 1.5 Hz); ¹³C{¹H} NMR (126 MHz, CDCl₃): 142.6, 139.6 (quintd, J=8.5, 2.6 Hz), 131.53 (m), 131.47. □ₘₐₓ (ATR-IR): 656 cm⁻¹ (br). HRMS (EI): calc'd for C₆H₄ClF₅Te [M]⁺: 335.8978, found: 335.8967.

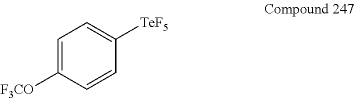

The reaction was run according to the general procedure. Colorless oil. ¹⁹F NMR (377 MHz, CDCl₃): −37.42 (1F, quint, J=152.0 Hz), −51.96 (4F, d, J=152.0 Hz), −57.61 (3F, s); ¹H NMR (400 MHz, CDCl₃): 8.01 (2H, d, J=8.9 Hz), 7.55 (2H, dm, J=8.9 Hz); ¹³C{¹H} NMR (101 MHz, CDCl₃): 154.1 (q, J=2.2 Hz), 138.7 (quintd, J=9.2, 2.9 Hz), 132.6 (quint, J=2.5 Hz), 122.7 (m), 120.1 (q, J=262.2 Hz). □ₘₐₓ (ATR-IR): 672 cm⁻¹ (br). HRMS (EI): calc'd for C₇H₄OF₈Te [M]⁺: 385.9191, found: 385.9192.

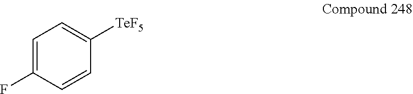

The reaction was run according to the general procedure. Light yellow oil. ¹⁹F NMR (282 MHz, CDCl₃): −37.02 (1F, quint, J=151.7 Hz), −51.94 (4F, d, J=151.7 Hz), −98.44 (1F, m); ¹H NMR (300 MHz, CDCl₃): 7.97 (2H, dd, J=8.9, 4.7 Hz), 7.43 (2H, m); ¹³C{¹H} NMR (76 MHz, CDCl₃): 166.5 (d, J=260.1 Hz), 136.6 (m), 133.1 (dquint, J=9.7, 2.5 Hz), 118.8 (dquint, J=23.1, 1.7 Hz). □ₘₐₓ (ATR-IR): 666 cm⁻¹ (br). HRMS (EI): calc'd for C₆H₄F₆Te [M]⁴: 319.9274, found: 319.9273.

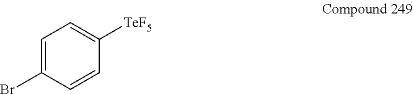

The reaction was run according to the general procedure. Waxy white solid. ¹⁹F NMR (377 MHz, CDCl₃): −37.27 (1F, quint, J=151.8 Hz), −52.28 (4F, d, J=151.8 Hz); ¹H NMR (400 MHz, CDCl₃): 7.87 (2H, dquint, J=8.8, 1.5 Hz), 7.79 (2H, d, J=8.8 Hz); ¹³C{¹H} NMR (101 MHz, CDCl₃): 140.3 (quintd, J=8.8, 2.9 Hz), 134.4 (m), 131.5 (quint, J=2.3 Hz), 131.1. □ₘₐₓ (ATR-IR): 654 cm⁻¹ (br). HRMS (EI): calc'd for C₆H₄BrF₅Te [M]⁺: 379.8473, found: 379.8453.

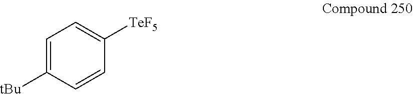

The reaction was run according to the general procedure. Note that we were unable to isolate an analytically pure sample. White solid. ¹⁹F NMR (377 MHz, CDCl₃): −36.49 (1F, quint, J=150.8 Hz), −53.11 (4F, d, J=150.8 Hz); ¹H NMR (400 MHz, CDCl₃): 7.83 (2H, d, J=8.8 Hz), 7.71 (2H, dquint, J=8.8, 1.7 Hz), 1.37 (9H, s). □ₘₐₓ (ATR-IR): 661 cm⁻¹ (br). HRMS (EI) calc'd for C₁₀H₁₃F₅Te [M]⁺: 357.9994, found: 357.9987.

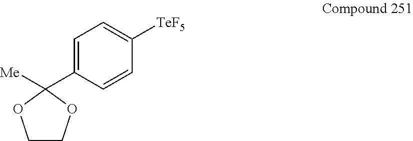

The reaction was run according to the general procedure. White solid; m.p. 86.2-86.9° C. ¹⁹F NMR (377 MHz, CD₃CN): −37.57 (1F, quint, J=148.4 Hz), −54.25 (4F, d, J=148.4 Hz); ¹H NMR (400 MHz, CD₃CN): 8.00 (2H, d, J=8.7 Hz), 7.91 (2H, dquint, J=8.7, 1.8 Hz), 4.10-4.02 (2H, m), 3.80-3.71 (2H, m), 1.63 (3H, s); ¹³C{¹H} NMR (101 MHz, CD₃CN): 153.5, 141.2 (quintd, J=5.9, 2.9 Hz), 131.2 (quint, J=2.2 Hz), 129.9 (quint, J=1.5 Hz), 108.5, 65.6, 27.4. □ₘₐₓ (ATR-IR): 661 cm⁻¹ (br). HRMS (EI): calc'd for C₉H₈O₂F₅Te [M]: 372.9501, found: 372.9502.

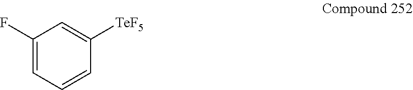

The reaction was run according to the general procedure. Colorless oil. ¹⁹F NMR (471 MHz, CD₃CN): −38.42 (1F, quint, J=149.4 Hz), −53.93 (4F, d, J=149.4 Hz), −106.22 (1F, m); ¹H NMR (500 MHz, CD₃CN): 7.93-7.84 (3H, m), 7.72-7.69 (3H, m); ¹³C{¹H}NMR (126 MHz, CD₃CN): 164.0 (dquint, J=255.1, 2.7 Hz), 141.9-141.5 (m), 134.7 (dquint, J=8.2, 1.8 Hz), 127.7-127.6 (m), 124.9 (d, J=20.9 Hz), 118.9 (dm, J=26.3). □ₘₐₓ (ATR-IR): 672 cm⁻¹ (br).

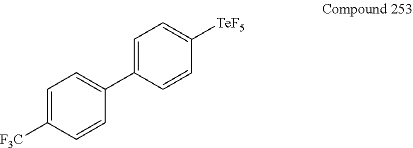

The reaction was run according to the general procedure. White solid; m.p. 127.6-128.6° C. ¹⁹F NMR (377 MHz, CD₃CN): −37.64 (1F, quint, J=148.3 Hz), −54.03 (4F, d, J=148.3 Hz), −63.10 (3F, s); ¹H NMR (400 MHz, CD₃CN): 8.16-8.10 (4H, m), 7.92 (2H, dm, J=8.4 Hz), 7.87 (2H, dm, J=8.4 Hz). □ₘₐₓ (ATR-IR): 665 cm⁻¹ (br)

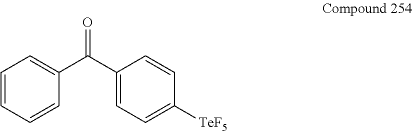

The reaction was run according to the general procedure. White solid; m.p. 94.2-96.4° C. ¹⁹F NMR (377 MHz, CD₃CN): −38.28 (1F, quint, J=148.6 Hz), −54.16 (4F, d, J=148.6 Hz); ¹H NMR (400 MHz, CD₃CN): 8.16 (2H, br d, J=8.6 Hz), 8.10 (2H, dquint, J=8.6, 1.7 Hz), 7.84-7.81 (2H, m), 7.73 (1H, tm, J=7.5 Hz), 7.61-7.56 (2H, m); ¹³C{H} NMR (101 MHz, CD₃CN): 195.3, 145.4, 144.5-144.2 (m), 136.9, 134.7, 133.3 (quint, J=1.5 Hz), 131.5 (quint, J=2.2 Hz), 131.07, 129.7. □ₘₐₓ (ATR-IR): 1664 cm⁻¹, 662 cm⁻¹ (br). HRMS (EI): calc'd for C₁₃H₉F₅OTe [M]⁺: 405.9630, found: 405.9632.

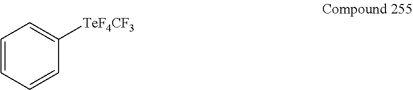

The reaction was run according to the general procedure. Light yellow oil. ¹⁹F NMR (377 MHz, CD3CN): −54.17 (3F, quint, J=21.8 Hz), −68.75 (4F, q, J=21.8 Hz); ¹H NMR (400 MHz, CD₃CN): 8.03 (2H, dm, J=8.2 Hz), 7.91 (1H, tm, J=7.5 Hz), 7.86-7.80 (2H, m); ¹³C{¹H} NMR (101 MHz, CD₃CN): 142.7 (quint, J=8.6 Hz), 137.0, 132.7, 131.1 (quint, J=2.2 Hz). Note: ¹³C NMR signal for “CF₃” was not resolved. □ₘₐₓ (ATR-IR): 625 cm⁻¹ (br).

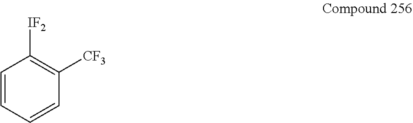

The reaction was run according to the general procedure. ¹H NMR (400 MHz, CD₃CN): δ=8.72 (1H, d, J=7.8 Hz), 8.06 (1H, d, J=7.7 Hz), 7.93 (1H, t, J=7.8 Hz), 7.85 (1H, t, J=7.8 Hz); ¹³C{¹H} NMR (101 MHz, CD₃CN): δ=140.3, 136.6-136.5 (m), 134.6 (t, J=1.8 Hz), 129.8 (q, J=32.6 Hz), 129.00 (q, J=5.4 Hz), 125.2 (q, J=273.7 Hz), 124.3 (tq, J=14.3, 1.7 Hz); ¹⁹F NMR (376 MHz, CD₃CN): δ=−60.36 (3F, s), −161.65 (2F, s).

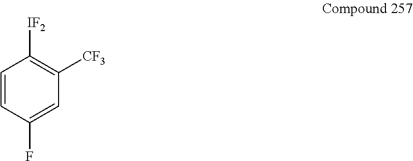

The reaction was run according to the general procedure. ¹H NMR (400 MHz, CD₃CN): δ=8.75 (1H, br dd, J=8.7, 5.1 Hz), 7.80 (1H, br d, J=8.7 Hz), 7.56 (1H, br t, J=7.3 Hz); ¹³C{¹H} NMR (101 MHz, CD₃CN): δ=165.1 (d, J=256.2 Hz), 143.5 (d, J=9.4 Hz), 133.1 (qd, J=33.7, 8.9 Hz), 123.5 (d, J=22.1 Hz), 123.0 (qd, J=273.9, 2.3 Hz), 119.7-119.1 (m), 117.7 (dq, J=27.0, 5.5 Hz); ¹⁹F NMR (376 MHz, CD₃CN): 5=−60.82 (3F, s), −103.17 (1F, s), −159.84 (2F, s).

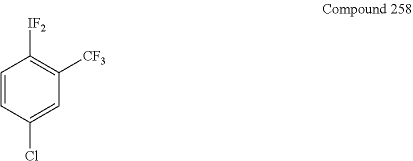

The reaction was run according to the general procedure. H NMR (500 MHz, CD₃CN): δ=8.67 (1H, d, J=8.5 Hz), 8.05 (1H, s.), 7.84 (1H, d, J=8.5 Hz); ¹³C{¹H} NMR (126 MHz, CD₃CN): =141.9, 140.6, 136.5, 131.8 (q, J=33.2 Hz), 129.6 (q, J=5.4 Hz), 123.2 (q, J=274.2 Hz), 122.2 (tm, J=14.7 Hz); ¹⁹F NMR (471 MHz, CD₃CN): δ=−60.77 (3F, s), −160.27 (2F, s)

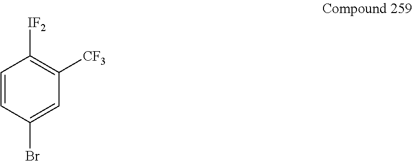

The reaction was run according to the general procedure. H NMR (500 MHz, CD₃CN): δ=8.58 (1H, d, J=8.4 Hz), 8.20 (1H, s), 8.00 (1H, d, J=8.5 Hz.); ¹³C{¹H} NMR (126 MHz, CD₃CN)=141.7, 139.5, 132.3 (q, J=5.3 Hz), 131.6 (q, J=33.1 Hz), 128.7 (t, J=2.1 Hz), 123.0 (q, J=274.3 Hz), 122.9-122.6 (m); ¹⁹F NMR (471 MHz, CD₃CN): δ=−60.70 (3F, s), −160.35 (2F, s).

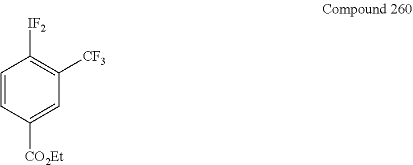

The reaction was run according to the general procedure. H NMR (400 MHz, CD₃CN): δ=8.80 (1H, d, J=8.2 Hz), 8.53 (1H, s), 8.37 (1H, d, J=8.2 Hz), 4.42 (2H, q, J=7.0 Hz), 1.39 (3H, t, J=7.1 Hz); ¹³C{¹H} NMR (101 MHz, CD₃CN): δ=164.6, 140.7, 136.9, 136.0, 130.4 (q, J=33.2 Hz), 129.4 (q, J=5.3 Hz), 127.4 (t, J=13.9 Hz), 123.5 (q, J=273.8 Hz), 63.2, 14.4; 1¹⁹F NMR (376 MHz, CD₃CN): δ=−60.62 (3F, s), −161.25 (2F, s).

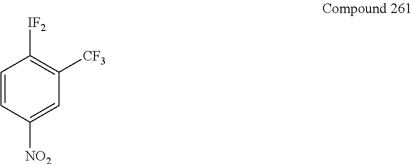

The reaction was run according to the general procedure. ¹H NMR (500 MHz, CD₃CN): δ=8.93 (1H, br d, J=8.6 Hz), 8.73 (1H, br s), 8.59 (1H, br d, J=8.5 Hz); ¹³C{¹H} NMR (126 MHz, CD₃CN): =150.8, 141.9, 131.7 (q, J=34.1 Hz), 131.3, 128.3 (t, J=14.4 Hz), 124.5 (q, J=5.5 Hz), 122.9 (q, J=274.3 Hz); ¹⁹F NMR (471 MHz, CD₃CN): δ=−60.88 (3F, s), −160.30 (2F, s).

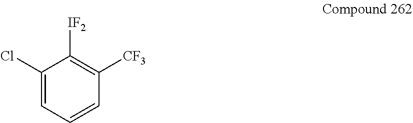

The reaction was run according to the general procedure. ¹H NMR (500 MHz, CD₃CN): δ=8.16 (1H, d, J=8.2 Hz), 7.95 (1H, d, J=7.8 Hz), 7.87 (1H, t, J=8.2 Hz); ¹³C{¹H} NMR (126 MHz, CD₃CN): δ=139.7, 136.5, 134.6, 133.0 (q, J=32.3 Hz), 130.2 (t, J=14.3 Hz), 127.5 (q, J=5.7 Hz), 123.6 (q, J=274.3 Hz); ¹⁹F NMR (471 MHz, CD₃CN): δ=−60.10 (3F, s), −163.36 (2F, s).

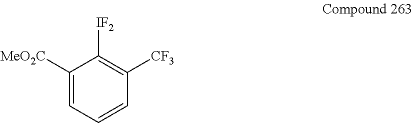

The reaction was run according to the general procedure. ¹H NMR (500 MHz, CD₃CN): δ=8.33 (1H, d, J=7.8 Hz), 8.22 (1H, d, J=7.9 Hz), 8.00 (1H, t, J=7.9 Hz), 4.03 (3H, s); ¹³C{¹H} NMR (126 MHz, CD₃CN): δ=166.6, 135.5, 135.2, 134.8, 132.2 (q, J=5.6 Hz), 131.9 (q, J=32.0 Hz), 124.1 (q, J=274.3 Hz), 123.4 (q, J=14.0 Hz), 54.4; ¹⁹F NMR (471 MHz, CD₃CN): 5=−59.23 (3F, s), −159.98 (2F, s).

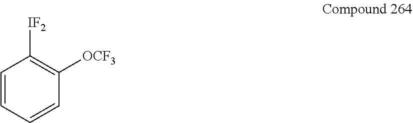

The reaction was run according to the general procedure. ¹H NMR (500 MHz, CD₃CN): δ=8.46 (1H, dd, J=8.0, 1.8 Hz), 7.82 (1H, t, J=8.0 Hz), 7.72 (1H, d, J=8.5 Hz), 7.56 (1H, t, J=7.8 Hz); ¹³C{¹H} NMR (126 MHz, CD₃CN) 5=146.4 (q, J=1.8 Hz), 137.9, 136.6, 130.5, 123.2 (t, J=13.8 Hz), 121.41 (q, J=259.8 Hz), 121.40 (q, J=1.9 Hz); ¹⁹F NMR (471 MHz, CD3CN): δ=−57.60 (3F, s), −166.40 (2F, s).

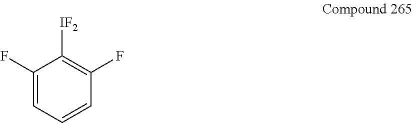

The reaction was run according to the general procedure. ¹H NMR (500 MHz, CD3CN): δ=7.80 (1H, t, J=7.7 Hz), 7.37 (2H, br s); ¹³C{¹H} NMR (126 MHz, CD₃CN): δ=160.0 (dd, J=253.9, 4.6 Hz), 138.7 (dd, J=11.2, 8.9 Hz), 113.5-113.2 (m), 108.4-107.6 (m); ¹⁹F NMR (471 MHz, CD₃CN): δ=−97.43 (2F, br. s), −165.78 (2F, s).

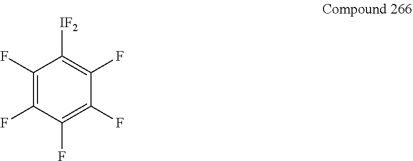

The reaction was run according to the general procedure. ¹⁹F NMR (282 MHz, CD₃CN): δ=−124.10 to −124.65 (2F, m), −145.92 (1F, tt, J=19.9, 5.1 Hz), −158.21 to −158.66 (2F, m), −162.08 (2F, s).

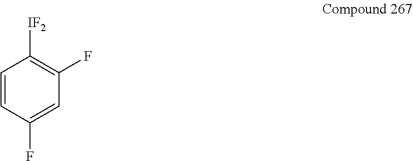

The reaction was run according to the general procedure. H NMR (500 MHz, CD₃CN): δ=8.37 (1H, dt, J=9.0, 4.6 Hz), 7.34 (1H, td, J=8.9, 2.8 Hz), 7.20 (1H, td, J=8.6, 2.7 Hz); ¹H{¹⁹F} NMR (500 MHz, CD₃CN): δ=8.37 (1H, d, J=8.9 Hz), 7.34 (1H, d, J=2.8 Hz), 7.20 (1H, td, J=9.0, 2.8 Hz); ¹³C{¹H} NMR (126 MHz, CD₃CN): δ=167.0 (ddt, J=256.1, 12.0, 1.9 Hz), 160.4 (dd, J=253.9, 13.3 Hz), 115.5 (dd, J=23.0, 3.4 Hz), 112.2 (dtd, J=23.3, 15.2, 4.5 Hz), 106.3 (t, J=26.8 Hz); ¹⁹F NMR (471 MHz, CD₃CN): δ=−94.80 (1F, d, J=11.4 Hz), −101.28 (1F, dt, J=11.1, 4.3 Hz), −165.09 (2F, s).

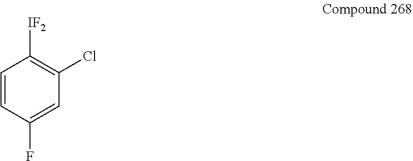

The reaction was run according to the general procedure. ¹H NMR (400 MHz, CD₃CN): δ=8.47 (1H, dd, J=8.9, 5.6 Hz), 7.64 (1H, dd, J=8.6, 2.8 Hz), 7.28 (1H, td, J=8.5, 2.8 Hz); ¹³C{¹H} NMR (101 MHz, CD₃CN): δ=160.0 (dt, J=256.6, 1.7 Hz), 140.7 (d, J=10.0 Hz), 138.6 (d, J=11.5 Hz), 127.6 (td, J=14.6, 4.0 Hz), 118.8 (t, J=26.7 Hz), 118.3 (t, J=22.7 Hz); ¹⁹F NMR (376 MHz, CD₃CN): δ=−103.50 (1F, tq, J=9.3, 4.8 Hz), −164.37 (2F, d, J=4.2 Hz); ¹⁹F{¹H} NMR (376 MHz, CD₃CN): δ=−103.50 (1F, t, J=4.5 Hz), −164.37 (2F, d, J=3.7 Hz).

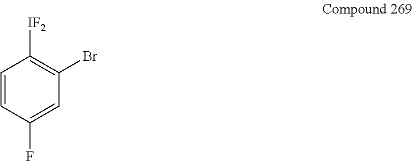

The reaction was run according to the general procedure. H NMR (400 MHz, CD₃CN): δ=8.47 (1H, dd, J=8.9, 5.5 Hz), 7.78 (1H, dd, J=8.8, 2.7 Hz), 7.36-7.25 (1H, m); ¹³C{¹H} NMR (101 MHz, CD₃CN): δ=165.5 (dt, J=257.4, 1.7 Hz), 141.1 (d, J=9.6 Hz), 130.7 (td, J=14.7, 4.0 Hz), 128.5 (d, J=10.4 Hz), 122.0 (t, J=26.3 Hz), 118.7 (t, J=22.7 Hz); ¹⁹F NMR (376 MHz, CD₃CN): δ=−103.74 (1F, br s), −163.35 (2F, br s).

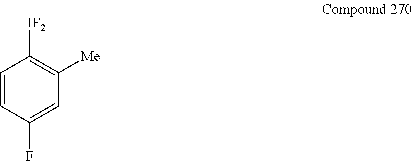

The reaction was run according to the general procedure. ¹H NMR (400 MHz, CD₃CN): δ=8.32 (1H, dd, J=8.9, 5.5 Hz), 7.34 (1H, dd, J=9.8, 3.0 Hz), 7.12 (1H, td, J=8.6, 3.1 Hz), 2.74 (3H, s); ¹³C{¹H} NMR (101 MHz, CD₃CN): δ=165.7 (dt, J=252.4, 1.9 Hz), 144.4 (d, J=9.5 Hz), 139.6 (d, J=9.5 Hz), 128.5 (td, J=13.6, 3.0 Hz), 118.9 (t, J=23.1 Hz), 116.8 (t, J=22.8 Hz), 25.1; 1¹⁹F NMR (376 MHz, CD₃CN): δ=−106.86 (1F, tt, J=9.9, 4.8 Hz), −168.31 (2F, d, J=3.7 Hz); ¹⁹F{¹H} NMR (376 MHz, CD₃CN): δ=−106.86 (1F, t, J=4.7 Hz), −168.32 (2F, d, J=4.2 Hz).

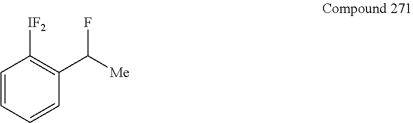

The reaction was run according to the general procedure. ¹H NMR (500 MHz, CD₃CN): δ=8.39 (1H, d, J=8.0 Hz), 7.85-7.77 (2H, m), 7.57 (1H, d, J=7.6 Hz), 6.07 (1H, dd, J=46.1, 6.4 Hz), 1.72 (3H, dd, J=24.1, 6.4 Hz); ¹³C{¹H} NMR (126 MHz, CD₃CN): 141.8 (d, J=20.9 Hz), 137.3, 134.7, 132.4, 129.4 (td, J=13.2, 4.3 Hz), 128.5 (d, J=7.7 Hz), 93.8 (d, J=129.7 Hz), 23.5 (d, J=25.2 Hz); ¹⁹F NMR (471 MHz, CD₃CN): 5=−165.35 (2F, s), −165.58 (1F, dq, J=47.8, 24.2 Hz).

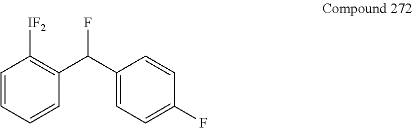

The reaction was run according to the general procedure. H NMR (500 MHz, CD₃CN): δ=8.42 (1H, d, J=8.0 Hz), 7.85-7.77 (2H, m), 7.61 (1H, br t, J=7.5 Hz), 7.45 (2H, br t, J=6.9 Hz), 7.17 (2H, br t, J=8.5 Hz), 7.02 (1H, d, J=46.1 Hz); ¹³C{¹H} NMR (126 MHz, CD₃CN): δ=163.6 (dd, J=246.6, 2.8 Hz), 139.6 (d, J=23.1 Hz), 138.1 (d, J=28.4 Hz), 137.6, 134.9 (dd, J=22.2, 3.2 Hz), 134.6, 132.8 (d, J=1.8 Hz), 130.6 (dd, J=8.7, 5.8 Hz), 129.7 (d, J=8.6 Hz), 116.6 (d, J=21.9 Hz), 95.4 (d, J=174.0 Hz); ¹⁹F NMR (471 MHz, CD₃CN): δ=−113.59 (1F, br. s), −161.74 (1F, d, J=46.2 Hz), −165.69 (2F, br. s)

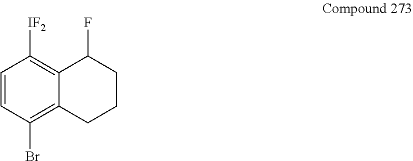

The reaction was run according to the general procedure. H NMR (500 MHz, CD₂Cl₂): δ=8.16 (1H, d, J=8.4 Hz), 7.79 (1H, dd, J=8.4, 2.1 Hz), 5.97 (1H, dt, J=49.3, 3.2 Hz), 3.12-3.05 (1H, m), 2.67-2.54 (1H, m), 2.50-2.41 (1H, m), 2.02-1.95 (2H, m), 1.92-1.82 (1H, m); ¹³C{¹H} NMR (126 MHz, CD₂Cl₂): 5=142.1, 136.4 (d, J=44.2 Hz), 135.3 (d, J=17.6 Hz), 132.0, 117.0, 88.4 (d, J=170.1 Hz), 31.4, 29.2 (d, J=21.5 Hz), 17.4, 2.1; 1¹⁹F NMR (471 MHz, CD₃CN): δ=−156.94 to −157.21 (1F, m), −165.33 (2F, s)

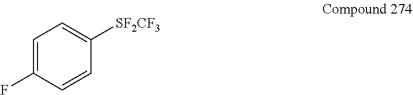

The reaction was run according to the general procedure. ¹⁹F NMR (282 MHz, CD₃CN): −13.31 (2F, qd, J=17.9, 2.0 Hz), −63.19 (3F, t, J=17.9 Hz), −106.82 to −106.95 (1F, m).

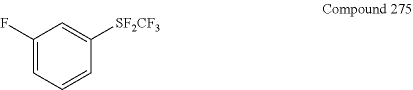

The reaction was run according to the general procedure. ¹⁹F NMR (282 MHz, CD₃CN): −13.15 (2F, q, J=18.2 Hz), −62.61 (3F, t, J=18.2 Hz), −110.66 to −110.80 (1F, m).

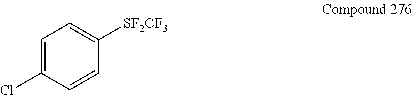

The reaction was run according to the general procedure. ¹⁹F NMR (282 MHz, CD₃CN): −13.80 (2F, q, J=18.0 Hz), −62.83 (3F, t, J=18.0 Hz).

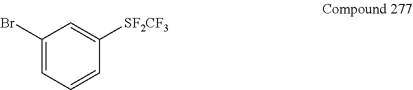

The reaction was run according to the general procedure. ¹⁹F NMR (282 MHz, CD₃CN): −13.30 (2F, q, J=18.3 Hz), −62.42 (3F, t, J=18.3 Hz).

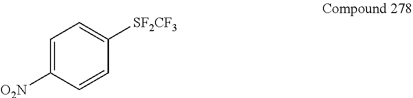

The reaction was run according to the general procedure. ¹⁹F NMR (282 MHz, CD₃CN): −11.89 (2F, q, J=18.0 Hz), −61.74 (3F, t, J=18.0 Hz).

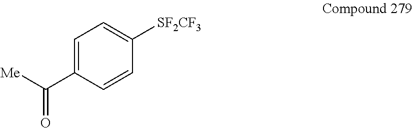

The reaction was run according to the general procedure. ¹⁹F NMR (282 MHz, CD₃CN): −13.24 (2F, q, J=18.1 Hz), −62.12 (3F, t, J=18.1 Hz).

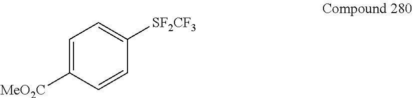

The reaction was run according to the general procedure. ¹⁹F NMR (282 MHz, CD₃CN): −13.22 (2F, q, J=18.3 Hz), −62.11 (3F, t, J=18.3 Hz).

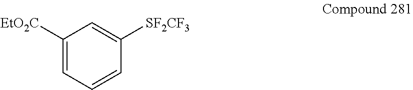

The reaction was run according to the general procedure. ¹⁹F NMR (282 MHz, CD₃CN): −14.10 (2F, q, J=18.0 Hz), −62.54 (3F, t, J=18.0 Hz).

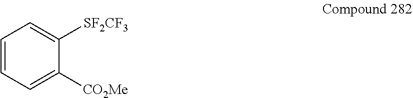

The reaction was run according to the general procedure. ¹⁹F NMR (282 MHz, CD₃CN): −4.73 (2F, q, J=17.6 Hz), −59.43 (3F, t, J=17.6 Hz).

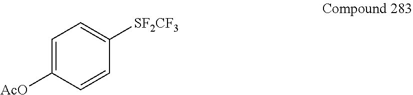

The reaction was run according to the general procedure. ¹⁹F NMR (282 MHz, CD₃CN): −13.66 (2F, q, J=17.9 Hz), −63.06 (3F, t, J=17.9 Hz).

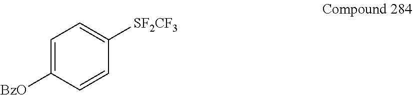

The reaction was run according to the general procedure. ¹⁹F NMR (282 MHz, CD₃CN): −13.46 (2F, q, J=18.2 Hz), −62.67 (3F, t, J=18.2 Hz).

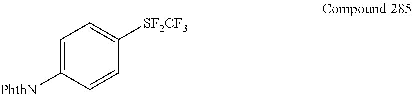

The reaction was run according to the general procedure. ¹⁹F NMR (282 MHz, CD₃CN): −14.06 (2F, q, J=18.3 Hz), −62.80 (3F, t, J=18.3 Hz).

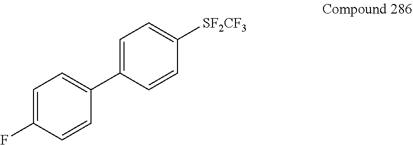

The reaction was run according to the general procedure. ¹⁹F NMR (282 MHz, CD3CN): −14.50 (2F, q, J=18.3 Hz), −62.91 (3F, t, J=18.3 Hz), −114.82 to −114.97 (1F, m).

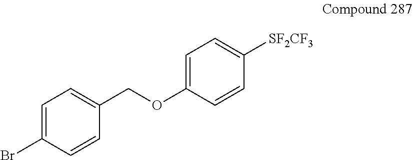

The reaction was run according to the general procedure. ¹⁹F NMR (282 MHz, CD₃CN): −14.36 (2F, q, J=18.0 Hz), −63.62 (3F, t, J=18.0 Hz).

1. A process for preparing a polyfluorinated compound of formula
Ar—R₁ (I),
wherein Ar—R₁ (I) is an aromatic ring system 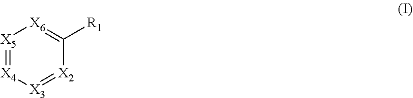
wherein
R₁ is selected from the group consisting of SF₄Cl, SF₃, SF₂CF₃, TeF₅, TeF₄CF₃, SeF₃, IF₂, SeF₂CF₃ and IF₄,
X₂ is N or CR₂,
X₃ is N or CR₃,
X₄ is N or CR₄,
X₅ is N or CR₅,
X₆ is N or CR₆, and
the total number of nitrogen atoms in the aromatic ring system is between 0 and 3,
wherein R₂, R₃, R₄, R₅ and R₆ are independently selected from the group consisting of hydrogen, fluoro, chloro, bromo, nitro, trifluoromethyl, 2,2,2-trifluoroethyl, pentafluorosulfanyl, phthalimido, azido, benzyloxy, trifluoromethoxy, 2,2,2-trifluoroethoxy, methoxycarbonyl, ethoxycarbonyl, methylcarbonyl, ethylcarbonyl, acetoxy, t-butyl, phenylcarbonyl, benzylcarbonyl, 3-trifluoromethylphenyl, phenylsulfonyl, methylsulfonyl, chlorophenyl, methyldoxolonyl, methyl, isopropyl, cyclopropyl, cyclobutyl, cyclopentyl, cyclohexyl, fluoromethyl, fluoroethyl and phenyl,
or if X₅ is CR₅ and X₆ is CR₆R₅ and R₆ may form together a saturated or unsaturated five or six membered ring system comprising one or more nitrogen, wherein the five or six membered ring system may be substituted with one or more residues R₇ having the same definition as R₂ to R₆, and
with the proviso that
if R₁ is SF₃, at least one of R₂ and R₆ is neither hydrogen nor fluoro and if R₁ is not SF₃, R₂ and R₆ are independently from each other either hydrogen or fluoro and if at least one of X₂, X₃, X₄, X₅ and X₆ is nitrogen, at least one of R₂, R₃, R₄, R₅ and R₆ is not hydrogen
the process involving the following reaction step
reacting a starting material selected from the group consisting of
Ar₂S₂, Ar₂Te₂, Ar₂Se₂, ArSCF₃, ArTeCF₃, ArI, ArSeCF₃, ArSCH₃, and ArSCl,
wherein Ar has the same definition as above,
with trichloroisocyanuric acid (TCICA) of the formula 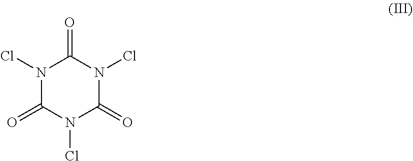 [SMILES: O=c1n(Cl)c(=O)n(Cl)c(=O)n1Cl]
in the presence of an alkali metal fluoride (MF).

2. The process for preparing a polyfluorinated compound according to claim 1
wherein Ar—R₁ (I) is an aromatic ring system 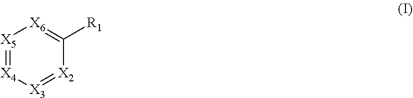
wherein
R₁ is selected from the group consisting of SF₄Cl, SF₃, SF₂CF₃, TeF₅, TeF₄CF₃, SeF₃, and IF₂,
X₂ is N or CR₂,
X₃ is N or CR₃,
X₄ is N or CR₄,
X₅ is N or CR₅,
X₆ is N or CR₆, and
the total number of nitrogen atoms in the aromatic ring system is between 0 and 3,
wherein R₂, R₃, R₄, R₅ and R₆ are independently selected from the group consisting of hydrogen, fluoro, chloro, bromo, nitro, trifluoromethyl, 2,2,2-trifluoroethyl, pentafluorosulfanyl, phthalimido, azido, benzyloxy, trifluoromethoxy, 2,2,2-trifluoroethoxy, methoxycarbonyl, ethoxycarbonyl, acetoxy, t-butyl and phenyl, and
with the proviso that
if R₁ is SF₃, at least one of R₂ and R₆ is neither hydrogen nor fluoro and if R₁ is not SF₃, R₂ and R₆ are independently from each other either hydrogen or fluoro and if at least one of X₂, X₃, X₄, X₅ and X₆ is nitrogen, at least one of R₂, R₃, R₄, R₅ and R₆ is not hydrogen
the process involving the following reaction step
reacting a starting material selected from the group consisting of
Ar₂S₂, Ar₂Te₂, Ar₂Se₂, Ar—SCF₃ and ArI,
wherein Ar has the same definition as above,
with trichloroisocyanuric acid (TCICA) of the formula 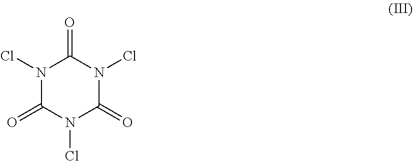 [SMILES: O=c1n(Cl)c(=O)n(Cl)c(=O)n1Cl]
in the presence of an alkali metal fluoride (MF).

3. The process according to claim 1, wherein the process is carried out in the presence of a catalytic amount of a Brønsted or Lewis acid.

4. The process according to claim 3, wherein the catalytic amount of the Brønsted or Lewis acid is between 5 mol % and 15 mol %.

5. The process according to claim 1, wherein the molar ratio of TCICA:MF is between 1:1 and 1:10.

6. The process according to claim 1 for preparing a polyfluorinated compound of formula Ar—R₁ (I).

7. The process according to claim 1, wherein R₁ is SF₄Cl or SF₃.

8. The process according to claim 1, wherein the aromatic ring system is a substituted or unsubstituted phenyl ring and R₁ to R₆ have the same definition as in claim 1.

9. The process according to claim 1, wherein at least one of X₂, X₃, X₄, X₅ and X₆ is nitrogen.

10. The process according to claim 8, wherein exactly two of X₂, X₃, X₄, X₅ and X₆ are nitrogen.

11. The process according to claim 1, wherein at least one of R₂, R₃, R₄, R₅ and R₆ is fluoro, chloro, bromo, methoxycarbonyl, ethoxycarbonyl or acetoxy.

12. The process according to claim 1, wherein the starting material is a diaryl dichalcogenide or a diheteroaryl dichalcogenide selected from the group consisting of Ar₂S₂, Ar₂Te₂ and Ar₂Se₂.

13. The process according to claim 1, wherein the starting material is Ar—SCF₃ or ArI.

14. The process according to claim 1 by reacting Ar—SF₄Cl in a second reaction step to obtain a compound of formula (V) or (VI) 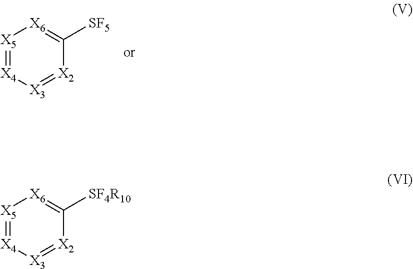
wherein
X₂ is N or CR₂,
X₃ is N or CR₃,
X₄ is N or CR₄,
X₅ is N or CR₅,
X₆ is N or CR₆, and
the total number of nitrogen atoms in the aromatic ring system is between 0 and 3,
R₂ and R₆ are independently from each other either hydrogen or fluoro and
R₃, R₄, and R₅ are independently selected from the group consisting of hydrogen, fluoro, chloro, bromo, nitro, trifluoromethyl, 2,2,2-trifluoroethyl, pentafluorosulfanyl, phthalimido, azido, benzyloxy, trifluoromethoxy, 2,2,2-trifluoroethoxy, methoxycarbonyl, ethoxycarbonyl, acetoxy, t-butyl and phenyl, and
R₁₀ is linear or branched, substituted or unsubstituted alkyl, α-alkenyl or α-alkynyl having 2 to 10 carbon atoms.

15. A compound of formula 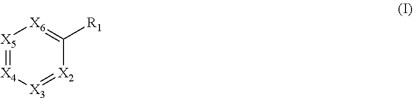
selected from the group consisting of

| Compound No. | R₁ | X₂ | X₃ | X₄ | X₅ | X₆ |
|---|---|---|---|---|---|---|
| 101 | SF₄Cl | C—H | C—H | C—NO₂ | C—COOMe | C—H |
| 102 | SF₄Cl | C—H | C—H | C—H | C—COOEt | C—H |
| 103 | SF₄Cl | C—H | C—H | C—OAc | C—H | C—H |
| 104 | SF₄Cl | C—H | C—H | C—NPhth | C—H | C—H |
| 105 | SF₄Cl | C—H | C—H | C—OCF₃ | C—H | C—H |
| 106 | SF₄Cl | C—H | C—H | C—SF₅ | C—H | C—H |
| 107 | SF₄Cl | N | C—H | C—COOMe | C—H | C—H |
| 108 | SF₄Cl | N | N | C—Ph | C—Ph | N |
| 109 | SF₅ | C—H | C—H | C—NO₂ | C—COOMe | C—H |
| 111 | SF₅ | C—H | C—H | C—OAc | C—H | C—H |
| 112 | SF₅ | C—H | C—H | C—NO₂ | C—COOH | C—H |
| 116 | SF₅ | C—H | C—H | C—OCF₃ | C—H | C—H |
| 118 | SF₅ | N | C—H | C—COOMe | C—H | C—H |
| 119 | SF₅ | N | N | C—Ph | C—Ph | N |
| 120 | SF₅ | N | C—H | C—COOH | C—H | C—H |
| 121 | SF₄Cl | C—H | O—Bz | C—H | C—H | C—H |
| 122 | SF₄Cl | C—H | C—H | O—Bz | C—H | C—H |
| 123 | SF₄Cl | C—F | O—Bz | C—H | C—H | C—H |
| 124 | SF₄Cl | C—F | C—H | O—Bz | C—H | C—H |
| 125 | SF₄Cl | C—F | C—H | C—H | O—Bz | C—H |
| 126 | SF₅ | C—H | O—Bz | C—H | C—H | C—H |
| 127 | SF₅ | C—F | O—Bz | C—H | C—H | C—H |
| 128 | SF₅ | C—F | C—H | C—H | O—Bz | C—H |
| 129 | SF₄Cl | C—H | C—N₃ | C—H | C—H | C—H |
| 130 | SF₄Cl | C—H | C—H | C—N₃ | C—H | C—H |
| 131 | SF₄Cl | C—F | C—N₃ | C—H | C—H | C—H |
| 132 | SF₄Cl | C—F | C—H | C—N₃ | C—H | C—H |
| 133 | SF₄Cl | C—F | C—H | C—H | C—N₃ | C—H |
